(19) United States
(12) Patent Application Publication
(10) Pub. No.: US 2026/0258176 A1
(43) Pub. Date: Sep. 3, 2026

(54) FLUORINATED ZWITTERIONIC POLYMERS AND METHODS OF USE

(71) Applicant: CORNELL UNIVERSITY, Ithaca, NY (US)

(72) Inventors: Shaoyi Jiang, Ithaca, NY (US); Sean Bailey, Ithaca, NY (US)

(21) Appl. No.: 18/877,416

(22) PCT Filed: Jun. 20, 2023
(86) PCT No.: PCT/US2023/025701

§ 371 Date: Dec. 20, 2024

Related U.S. Application Data
(60) Provisional application No. 63/353,707, filed on Jun. 20, 2022.

(51) Int. Cl. C08F 220/36; C07C 229/12; C09D 4/00; C09D 5/16

(52) U.S. Cl. CPC C08F 220/36; C07C 229/12; C09D 4/00; C09D 5/1637

(57) ABSTRACT

Fluorinated monomer compositions having the formula: A-L-Z (1), wherein A is a polymerizable group (e.g., a vinyl group); L is a bond or linking portion; Z is a zwitterionic moiety or a zwitterionic precursor moiety containing a protecting group capable of deprotection to form a charged group; wherein at least one hydrogen atom in A, L, and/or Z in Formula (1) is substituted by a fluorinated hydrocarbon group, wherein the fluorinated hydrocarbon group contains 1-12 carbon atoms and precisely or at least one, two, three, four, five, or six fluorine atoms. Also described herein are fluorinated zwitterionic polymers derived from such monomers. Also described herein are mixed-charge zwitterionic polymers. Other aspects include methods for producing the monomers and polymers, bulk material (e.g., hydrogel or copolymer hybrid) compositions of any of the zwitterionic polymers described herein, fouling-resistant and/or fouling releasing surfaces and objects, and nanoparticles containing the polymer or bulk material.

### CROSS REFERENCE TO RELATED APPLICATIONS

This application claims the benefit of priority from U.S. Provisional Application No. 63/353,707, filed on Jun. 20, 2022, which is herein incorporated by reference in its entirety.

### STATEMENT REGARDING FEDERALLY SPONSORED RESEARCH OR DEVELOPMENT

This invention was made with government support under Department of Navy award N00014-20-1-2731 and N00014-23-1-2186 issued by the Office of Naval Research. The government has certain rights in the invention.

### FIELD OF THE INVENTION

The present invention generally relates to fluorinated zwitterionic polymeric materials, particularly those having a non-fouling, foul-resistant, or fouling-release property. The present invention more specifically relates to zwitterionic polymeric materials containing a poly(carboxybetaine) (pCB), poly(sulfobetaine) (pSB), poly(trimethylamine N-oxide) (pTMAO), or poly(phosphorylcholine) (pPC) component.

### BACKGROUND

Zwitterionic materials, such as poly(carboxybetaine) (pCB), poly(sulfobetaine) (pSB), poly(trimethylamine N-oxide) (pTMAO), and poly(phosphorylcholine) (pPC), have gained particular attention since they can effectively resist non-specific adsorption and fouling from biomolecules and microorganisms. Although conventional zwitterionic materials may possess good non-fouling or foul-resistant abilities, they generally lack a fouling-release property. Material compositions containing both foul-resistant and fouling-releasing abilities are highly desired but have remained elusive. There is yet a further need for such materials also having a better fouling-release ability.

### SUMMARY

The present disclosure is foremost directed to novel fluorinated zwitterionic polymer compositions having outstanding non-fouling (fouling resistant) ability. The polymer compositions described herein also surprisingly exhibit exceptional fouling-release ability.

Zwitterionic polymers have been shown to possess excellent antifouling performance due to their ability to tightly bind water and construct an energy barrier to the adsorption of foulants. However, zwitterionic coatings have high surface energy which can lead to adhesion between foulant and coating upon dehydration of the polymer surface once foulants are attached. These issues could potentially be resolved via the integration of hydrophobic groups into a zwitterionic polymer to promote enhanced release of any foulant capable of circumventing the hydration layer. Amphiphilic coatings are primarily synthesized through copolymerization of hydrophilic and hydrophobic monomers. However, both non-fouling (e.g., from zwitterionic) and fouling-release (e.g., from polydimethylsiloxane (PDMS)) materials are compromised. Construction of an amphiphilic monomer has proven to be challenging, especially one that utilizes a zwitterionic moiety as its hydrophilic component and fluorinated moiety as its hydrophobic component. Derivatizing a zwitterionic monomer with hydrophobic features, particularly fluorinated moiety, however, could be highly rewarding as its chemical ambiguity would exist on a molecular scale (e.g., below 1 nm), permitting it to resist the adsorption of even the smallest protein. Such an amphiphilic polymer may be designed with two key strategies in mind: the capability to undergo surface reorganization and the possession of atomic scale chemical ambiguity. The first strategy permits the coating to maintain a strong hydration layer in water to resist adsorption but switch to a low energy conformation upon dehydration that promotes foulant release. The second strategy is because amphiphilic structures below the dimensions of the smallest adhesive biomacromolecules can inevitably lead to weak adhesion of any foulant. Thus, this unique molecule can be achieved by the fluorination of various zwitterionic monomers in the different positions of each monomer.

The zwitterionic polymer can be fluorinated, typically by substitution with a fluorinated hydrocarbon, on the backbone of the polymer or on a pendant portion of the polymer. The zwitterionic polymer may be a homopolymer or copolymer, such as a block copolymer. By being zwitterionic, the polymer may contain a zwitterionic moiety (combination of positively and negatively charged groups) on a single pendant chain of the polymer, or the polymer may contain positively and negatively charged groups on separate pendant chains of the polymer which together form a zwitterionic system (i.e., mixed-charge zwitterionic copolymer). The zwitterionic moiety (Z) may be selected from, for example, carboxybetaine (CB), sulfobetaine (SB), phosphobetaine (PB), and trialkylamine-N-oxide (TMAO) zwitterionic moieties. The present disclosure is also directed to monomer compositions useful in producing the polymer compositions.

In one aspect, the present disclosure is directed to zwitterionic monomer compositions that are fluorinated. The monomer composition may be within the scope of the following general formula: A-L-Z (1), wherein A is a polymerizable group (e.g., olefin, diacid, diester, diol, or diisocyanate); L is a bond or linking portion; Z is a zwitterionic moiety or a zwitterionic precursor moiety containing a protecting group capable of deprotection to form a charged group; wherein at least one hydrogen atom in A, L, and/or Z in Formula (1) is substituted by a fluorinated hydrocarbon group or fluorine atom, wherein the fluorinated hydrocarbon group contains 1-12 carbon atoms and precisely or at least one, two, or three fluorine atoms. In some embodiments, the fluorinated hydrocarbon group is partially fluorinated or perfluorinated. In some embodiments, for any of the formulas in this disclosure, the fluorinated hydrocarbon group is composed of only carbon, hydrogen, and fluorine atoms, or composed of only carbon and fluorine atoms, particularly when the fluorinated hydrocarbon group is bound directly or indirectly to an ammonium nitrogen. In some embodiments, for any of the formulas in this disclosure, the fluorinated hydrocarbon group does not contain an oxygen or nitrogen atom, particularly when the fluorinated hydrocarbon group is bound directly or indirectly to an ammonium nitrogen. In particular embodiments, for any of the formulas in this disclosure, the fluorinated hydrocarbon group has the formula —(CH₂)ₓ—(RF), wherein x is precisely or at least 1, 2, 3, 4, 5, or 6, and RF is a fluorinated hydrocarbon group.

The monomer may more particularly have the following structure:

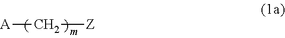

wherein A and Z are as defined under Formula (1); and m is an integer of at least 1 (or more particularly, 1-12, wherein the methylene groups may or may not be interrupted by a heteroatom-containing group, such as —O—, —NR′—, —C(O)O—, or —C(O)NR—); wherein at least one hydrogen atom in A, CH₂, and/or Z in Formula (1a) is substituted by a fluorinated hydrocarbon group or fluorine atom, wherein the fluorinated hydrocarbon group contains 1-12 carbon atoms and at least one, two, or three fluorine atoms. The group R′ may be a hydrogen atom or hydrocarbon group, wherein the hydrocarbon groups typically contain 1-12 carbon atoms.

The monomer may more particularly have the following olefin structure:

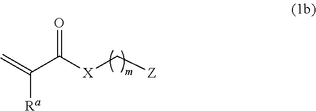

wherein: R^{a} is H or an alkyl group containing 1-3 carbon atoms; X is O or NR^{b}, wherein R^{b} is H or an alkyl group containing 1-3 carbon atoms; Z is a zwitterionic moiety or a zwitterionic precursor moiety containing a protecting group capable of deprotection to form a charged group; m is an integer of at least 1; and at least one hydrogen atom in Formula (1b) is substituted by a fluorinated hydrocarbon group or fluorine atom, wherein the fluorinated hydrocarbon group contains 1-12 carbon atoms and at least one, two, or three fluorine atoms.

The monomer may more particularly have the following structure:

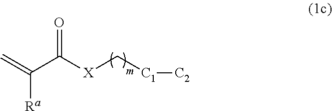

wherein R^{a}, X, and m are as defined under Formula (1b); and C₁ and C₂ are independently selected as positively charged and negatively charged groups to form a zwitterionic moiety C₁-C₂; provided that at least one hydrogen atom on R^{a}, X, methylene group under m, C₁, and/or C₂ is substituted by a fluorinated hydrocarbon group or fluorine atom, wherein the fluorinated hydrocarbon group contains 1-12 carbon atoms and at least one, two, or three fluorine atoms.

The monomer of Formula (1c) may more particularly have the following structure:

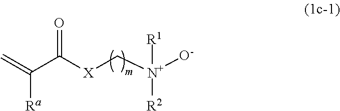

wherein R^{a}, X, and m are as defined under Formula (1b); and R¹ and R² are independently selected from R^{a}, as defined above; provided that at least one hydrogen atom on R^{a}, X, methylene group under m, R¹, and/or R² is substituted by a fluorinated hydrocarbon group or fluorine atom, wherein the fluorinated hydrocarbon group contains 1-12 carbon atoms and at least one, two, or three fluorine atoms.

Alternatively, Z may contain a positively charged group indirectly bound to a negatively charged group via a linker, such as in the following structure:

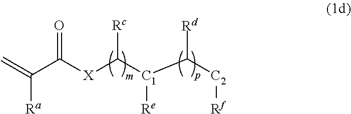

wherein R^{a}, X, and m are as defined under Formula (1b); R^{c}, R^{d}, R^{e}, and R^{f} are selected from hydrogen atom, hydrocarbon group, fluorine atom, and fluorinated hydrocarbon group containing 1-12 carbon atoms and at least one, two, or three fluorine atoms, wherein at least one of R^{c}, R^{d}, R^{e}, and R^{f} is said fluorinated hydrocarbon group or fluorine atom, wherein R^{c} and R^{d} are each independently present twice for each methylene linkage; one or both of R^{e} and R^{f} are alternatively selected from positive and negative charges; C₁ and C₂ are independently selected as positively charged and negatively charged groups to form a zwitterionic moiety; C₁ is optionally neutral and R^{e} is a protecting group capable of deprotection to form a charged group, or C₂ is optionally neutral and R^{f} is a protecting group capable of deprotection to form a charged group; and p is an integer of at least 1. The protecting group may be, for example, a bulky alkyl group (e.g., t-butyl) forming an ester with a carboxylate. Protecting groups may be removed by standard chemical means to produce a charged group to form a zwitterion. Thus, a monomer in which C₁ or C₂ contains a protecting group can be considered a zwitterionic monomer precursor. In some embodiments, C₁ or C₂ is a neutral amine group, which can be reacted with an amine-reactive compound, such as an alkyl halide, to result in a quaternary ammonium group. Thus, a monomer in which C₁ or C₂ is a neutral amine can also be considered a zwitterionic monomer precursor.

In more particular embodiments, the olefin monomer of Formula (1d) has the following structure:

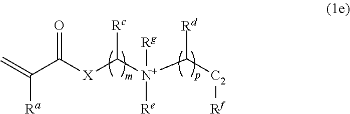

wherein R^{a}, X, and m are as defined above; R^{c}, R^{d}, R^{e}, R^{f}, and R^{g} are selected from hydrogen atom, hydrocarbon group, fluorine atom, and fluorinated hydrocarbon group containing 1-12 carbon atoms and at least one, two, or three fluorine atoms, wherein at least one of R^{c}, R^{d}, R^{e}, R^{f}, and R^{g} is said fluorinated hydrocarbon group or fluorine atom (except that a fluorine atom is generally not directly attached to nitrogen); R^{f} is alternatively a negative charge; C₂ is a negatively charged group; optionally, C₂ is neutral and R^{f} is a protecting group capable of deprotection to form a charged group; and p is an integer of at least 1. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a fluorinated hydrocarbon group.

Alternatively, the olefin monomer of Formula (1e) may more particularly have the following structure:

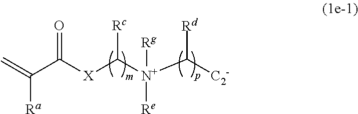

wherein R^{a}, X, and m are as defined above; R^{c}, R^{d}, R^{e}, and R^{g} are selected from hydrogen atom, hydrocarbon group, fluorine atom, and fluorinated hydrocarbon groups containing 1-12 carbon atoms and at least one, two, or three fluorine atoms, wherein at least one of R^{c}, R^{d}, R^{e}, R^{f}, and R^{g} is said fluorinated hydrocarbon group or a fluorine atom; C₂ is a negatively charged group; and p is an integer of at least 1. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a fluorinated hydrocarbon group.

The olefin monomer of Formula (1e-1) may more particularly have any of the following structures:

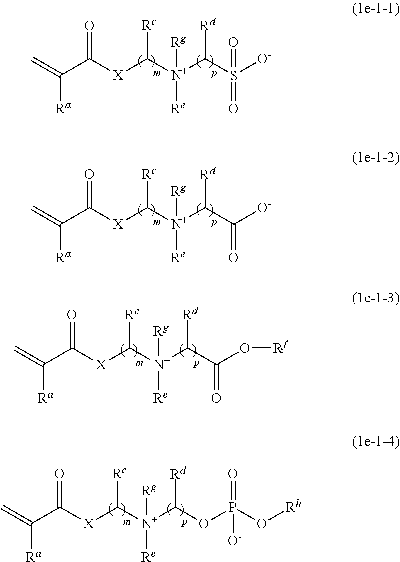

wherein R^{a}, X, and m are as defined above; R^{c}, R^{d}, R^{e}, R^{f}, R^{g}, and R^{h} are selected from hydrogen atom, hydrocarbon group, fluorine atom, and fluorinated hydrocarbon groups containing 1-12 carbon atoms and at least one, two, or three fluorine atoms, wherein at least one of R^{c}, R^{d}, R^{e}, R^{f}, R^{g}, and R^{h} is said fluorinated hydrocarbon group or fluorine atom; R^{f} is optionally a protecting group capable of deprotection to form a carboxylate group; and p is an integer of at least 1. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a fluorinated hydrocarbon group.

In other embodiments of Formula (1d), the olefin monomer composition has the following structure:

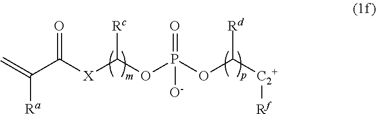

wherein R^{a}, X, and m are as defined above; C₂⁺ is a positively charged group; p is an integer of at least 1; and R^{c}, R^{d}, and R^{f} are selected from hydrogen atom, fluorine atom, hydrocarbon group, and fluorinated hydrocarbon group containing 1-12 carbon atoms and at least one, two, or three fluorine atoms, wherein at least one of R^{c}, R^{d}, and R^{f} is said fluorinated hydrocarbon group; wherein R^{f} is alternatively absent.

In particular embodiments of Formula (1f), the olefin monomer has the following structure:

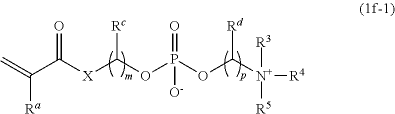

wherein R^{a}, X, and m are as defined above; p is an integer of at least 1; and R^{c}, R^{d}, R³, R⁴, and R⁵ are selected from hydrogen atom, hydrocarbon group, fluorine atom, and fluorinated hydrocarbon group containing 1-12 carbon atoms and at least one, two, or three fluorine atoms, wherein at least one of R^{c}, R^{d}, R³, R⁴, and R⁵ is said fluorinated hydrocarbon group or fluorine atom.

The present disclosure is also directed to polymers of any of the monomers described above in Formulas (1), (1a), (1b), (1c), (1c-1), (1d), (1e), (1e-1), (1e-1-1), (1e-1-2), (1e-1-3), (1e-1-4), (1f), and (1f-1). The polymers may be, for example, any of the polymers of the Formulas (I), (Ia), (Ib), (Ic), (Ic-1), (Id), (Ie), (Ie-1), (Ie-1-1), (Ie-1-2), (Ie-1-3), (Ie-1-4), (If), and (If-1), as depicted in the claims. The polymers may be homopolymers of any of the monomers described above, or the polymers may be copolymers containing any of the zwitterionic monomers described above copolymerized with each other or copolymerized with a non-zwitterionic monomer (e.g., vinyl alcohol, acrylate, methacrylate, trifluoroethylene, tetrafluoroethylene, styrene, or butadiene, or fluorinated versions of any one of these).

The present disclosure is also directed to mixed-charged zwitterionic copolymers comprising the following formula:

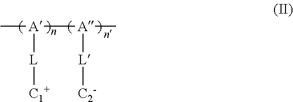

wherein: A′ and A″ are backbones of different copolymer segments shown in Formula (II); L and L′ are linking portions; C₁₇ is a positively charged group, and C₂ is a negatively charged group, wherein C₁⁺ and C₂⁻ together form a zwitterionic system; n and n′ are independently selected from integers of at least 2; wherein at least one hydrogen atom in A′, A″, L, L′, C₁⁺, and/or C₂⁻ in Formula (I) is substituted by a fluorinated hydrocarbon group or fluorine atom, wherein the fluorinated hydrocarbon group contains 1-12 carbon atoms and at least one, two, or three fluorine atoms. In some embodiments, A′ and A″ are olefinic. Notably, the polymer of Formula (II) may have any of the known copolymer arrangements, including block, alternating, periodic, and random arrangements. Moreover, although Formula (II) depicts a binary copolymer, the copolymer of Formula (II), may or may not contain one or more additional units or segments to result in a ternary or quaternary copolymer.

The present disclosure is also directed to bulk materials containing any of the above described fluorinated zwitterionic polymers. The bulk material may include any of the fluorinated zwitterionic polymers disclosed in this application, including, for example, Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II). In one instance, the bulk material is a copolymer hybrid containing the fluorinated zwitterionic monomer units or a fluorinated zwitterionic polymer segment attached to (e.g., grafted onto) or integrated into a conventional polymer, such as, for example, PDMS, PP, and PVC. The resulting copolymer hybrid may be, for example, a block, random, alternating, branched, or star copolymer. In another instance, the bulk material includes hydrogels, including single, double, and triple network entanglement hydrogels containing one, two, or three, respectively, of any of the zwitterionic polymers described in this disclosure. In particular embodiments, the hydrogel is a single (one-component) hydrogel containing any of the fluorinated zwitterionic polymers or copolymer described above. The hydrogel composition may be a chemically (i.e., covalently) crosslinked version of one, two, or three of the zwitterionic polymers and/or copolymer hybrids described herein, and may or may not be an entangled network. In other embodiments, the hydrogel composition may be a single, double, or triple network entanglement of one, two or three zwitterionic polymers selected from any of the polymer compositions disclosed herein, including those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), and sub-formulas thereof. In some embodiments, the hydrogel composition is a zwitterionic double-network (ZDN) hydrogel composition. In other embodiments, the hydrogel composition is a zwitterionic triple-network (ZTN) hydrogel composition. In particular embodiments, one, two, or all three of the zwitterionic polymers in the network are derived from olefinic monomers.

The present disclosure is also directed to a method for rendering a surface fouling-resistant and/or fouling-releasing, the method comprising coating the surface with any of the polymer compositions disclosed herein, including those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), and sub-formulas thereof, or a crosslinked hydrogel, or a hydrogel network entanglement of any one, two or three of these polymer compositions, or a copolymer hybrid, or a nanoparticle composition, as described below. The surface may be part of an object designed to operate in a marine environment (e.g., ship hull) or medical environment (e.g., catheter, surgical device, or implant).

The present disclosure is also directed to a fouling-resistant and/or fouling-release object comprising: (i) an object having a surface; and (ii) a coating comprising any one, two, or three polymer compositions disclosed herein, including those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), and sub-formulas thereof. The coating may also be a bulk material, as described above, such as a copolymer, a crosslinked hydrogel, or a hydrogel network entanglement of any one, two or three of these polymer compositions on the surface. The coating may also be a nanoparticle composition of any one, two, or three polymer compositions described herein. In some embodiments, the surface of the object possesses both a fouling-resistant and a fouling-release ability.

The present disclosure is also directed to zwitterionic nanoparticle compositions comprising any of the polymer compositions and bulk materials thereof disclosed herein, wherein the polymer may be any of those described herein, including those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), and sub-formulas thereof. The bulk material may be, for example, a copolymer hybrid, a crosslinked hydrogel, or a hydrogel network entanglement of any one, two or three of these polymer compositions within and/or on surfaces of nanoparticles. The nanoparticles may have a size of, for example, 1, 5, 10, 20, 50, 100, 200, or 500 nm, or a size within a range between any two of these values.

In some embodiments, the zwitterionic hydrogel compositions are zwitterionic triple-network (ZTN) hydrogel compositions containing a network entanglement of first, second, and third zwitterionic polymers. The ZTN hydrogels have both high mechanical strength and excellent non-fouling and fouling-release properties in saline environments. Typically, at least the second and third zwitterionic polymers contain at least 30, 40, or 50 mol % zwitterionic moieties. The first zwitterionic polymer may contain at least, for example, 1, 2, 3, 4, 5, 10, 20, or 30 mol % zwitterionic moieties. In some embodiments, each of the first, second, and third zwitterionic polymers (or at least the second and third zwitterionic polymers) contains at least 50, 60, or 70 mol % zwitterionic moieties.

In yet another aspect, the present disclosure is directed to a method of preventing or reducing the rate of fouling of a surface, or rendering a surface fouling resistant, which may also include a foul-releasing property. In some embodiments, the method includes coating the surface with any one of the polymers described above, which may be or include any of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), or a bulk material composition (e.g., copolymer hybrid, crosslinked hydrogel, or hydrogel network entanglement) of any one, two or three of the polymer compositions or a nanoparticle composition containing any one, two, or three of the polymers described above.

In some cases, a zwitterionic single-network (ZSN) hydrogel is produced by coating the surface with a first zwitterionic polymer or a copolymer thereof, followed by absorbing a precursor solution containing monomers of the same type that produce the first zwitterionic polymer, followed by polymerizing the monomers to result in the same type of zwitterionic polymer entangled within itself. In other embodiments, a ZSN hydrogel is produced by crosslinking (e.g., by chemical or radiative methods) a single zwitterionic polymer, which may be any of the zwitterionic polymers described in this disclosure. For producing zwitterionic double network and triple network hydrogels on a surface, the method may include at least the following steps: (i) coating the surface with a first zwitterionic polymer or copolymer thereof; (ii) absorbing a first precursor solution into the first zwitterionic polymer, wherein the first precursor solution includes a first zwitterionic monomer species (and optionally, a non-zwitterionic monomer species) dissolved in a solvent; (iii) polymerizing the first zwitterionic monomer species (and optionally, non-zwitterionic species) to form a second zwitterionic polymer (or copolymer) while absorbed in the first zwitterionic polymer to form a zwitterionic double-network (ZDN) hydrogel composition containing the second zwitterionic polymer or copolymer entangled in the first zwitterionic polymer; (iv) absorbing a second precursor solution into the ZDN hydrogel composition, wherein the second precursor solution includes a second zwitterionic monomer species dissolved in a solvent; and (v) polymerizing the second zwitterionic monomer species (and optionally, a non-zwitterionic monomer species) to form a third zwitterionic polymer while absorbed in the ZDN hydrogel composition to form the zwitterionic triple-network (ZTN) hydrogel composition containing the third zwitterionic polymer entangled in the ZDN hydrogel composition; wherein at least the second and third zwitterionic polymers typically contain at least 50 mol % zwitterionic moieties. In different embodiments, at least the second and third zwitterionic polymers in the Zwitterionic Triple-Network (ZTN) hydrogels comprise 100, 90, 80, 70, 60, or 50 mol % zwitterionic moieties. In other embodiments, the three polymer networks independently comprise 100, 90, 80, 70, 60, or 50 mol % zwitterionic moieties.

The coated object is designed to be fouling resistant particularly in a saline environment. The saline environment is typically ocean water, but may be another type of brackish water, either from a natural or industrial source. When a ZTN hydrogel coating is used, the first, second, and third polymer networks may comprise more than 50 mol % of zwitterionic (Z) moieties. The as-prepared first network is typically swellable. The precursor solution of the second network then forms the second network, which introduces the “lock effect” to the first network in the as-prepared double network (DN) hydrogel. The as-prepared DN hydrogel is immersed in the precursor solution of the third network, forming the third network, which protects the “lock effect” and strengthens the hydrogel in saline environments. The resulting fluorinated ZTN hydrogel shows macroscopic transparency, high stability, and remarkable mechanical strength and excellent non-fouling performance in water or saline environments and can be used in such applications as devices and coatings in the medical and marine fields.

In further embodiments, the hydrogels have one or more of the following properties after the hydrogels are soaked in water or saline environment (e.g., phosphate-buffered saline and seawater): compressive fracture stress (>0.5, 1, 3, 5, 10, 12, and 15 MPa) or a compressive fracture strain (>50%, 80%, and 99%) along with Young's modulus (>0.01, 0.1, 0.5 MPa, 1 MPa).

In further embodiments from any of the above embodiments, zwitterionic poly(sulfobetaine) (pSB) is used in the second and third zwitterionic polymer networks of a ZTN hydrogel. In further embodiments from any of the above embodiments, zwitterionic moieties can be, but not limited to, poly(carboxybetaine) (pCB), poly(sulfobetaine) (pSB), poly(phosphobetaine) (pPB), poly(phosphorylcholine) (pPC), poly(choline phosphate) (pCP), poly(trimethylamine-N-oxide) (pTMAO) or their latent derivatives. In further embodiments from any of the above embodiments, the first network is chemically or physically crosslinked. In further embodiments from any of the above embodiments, the second or third network is chemically or physically crosslinked. In further embodiments from any of the above embodiments, the zwitterionic moieties can be polymerized by, but not limited to, addition, condensation, ring-opening, and free radical polymerization. In further embodiments from any of the above embodiments, the physical crosslinking may include ionic bonding, hydrogen bonding, or dipole-dipole crosslinking.

In further embodiments from any of the above embodiments, the ZDN or ZTN hydrogel has a fibrinogen binding level of less than 20%, 15% or 10% relative to that of tissue culture polystyrene (TCPS) tested via a fibrinogen binding assay (polymer surface is incubated at 37° C. for 90 minutes with a 1.0 mg/mL fibrinogen solution in 0.15 M phosphate-buffered saline at pH 7.4).

In further embodiments from any of the above embodiments, the hydrogel has a fibrinogen binding level of less than 20%, 15% or 10% relative to that of tissue culture polystyrene (TCPS) tested via a fibrinogen binding assay (hydrogel is incubated at 37° C. for 1.5 h with a 1.0 mg/mL fibrinogen solution in 0.15 M phosphate-buffered saline at pH 7.4).

In further embodiments from any of the above embodiments, the hydrogels have an undiluted human serum binding level of less than 20%, 15% or 10% relative to that of tissue culture polystyrene (TCPS) tested via the BCA method (hydrogel can be incubated at 37° C. for 2 h in solution in undiluted human serum, then sonicated in phosphate-buffered saline+1 wt % sodium n-dodecyl sulfate (SDS) solution for 5 minutes to desorb proteins. This solution can be analyzed with Micro-BCA assay for quantifying the amount of adsorbed proteins).

In further embodiments from any of the above embodiments, the hydrogel has a water content (>50%, 70%, 80%, and 90%) and low swelling (the swelling ratio, (Mw-Md)/Md, is less than 300%, where Mw is the wet weight of the hydrogel and Md is the dry weight of the hydrogel soaked in DI water or 0.15 M phosphate-buffered saline at pH 7.4 until equilibrium or 36 g/L seawater).

In further embodiments from any of the above embodiments, the hydrogels can be prepared in a three-step method. The three-step method can be practiced as follows: the first network hydrogels comprising a zwitterionic polymer are formed first, the as-prepared first network hydrogels are soaked until equilibrium in the precursor solution of a second network comprising zwitterionic monomers, which are further polymerized to form the zwitterionic double-network (ZDN) hydrogels. After that, the as-prepared ZDN hydrogels are soaked until equilibrium in the precursor solution of a third network comprising zwitterionic monomers. The equilibrated ZDN hydrogels containing the precursor solution of the third network is further polymerized to form the zwitterionic triple-network (ZTN) hydrogels.

In further embodiments from any of the above embodiments, the first network hydrogels comprise a zwitterionic polymer that is highly swollen in the precursor solution of the second network. The swelling ratios ((Mw−Md)/Md) of the first network hydrogels in water are from 200% to 6000%, where Mw is the wet weight of the hydrogel and Md is the dry weight of the hydrogel soaked in DI water.

In further embodiments from any of the above embodiments, the ZTN hydrogels can be prepared in a three-step method, wherein three networks containing zwitterionic moieties are reacted independently and sequentially via chemical or physical crosslinking.

In further embodiments from any of the above embodiments, the zwitterionic double-network (ZDN) hydrogels can absorb a large number of the third network monomers. The third and second (3rd/2nd) network molar ratios in the zwitterionic triple-network (ZTN) hydrogels may be from 0.2 to 5.

The polymers or bulk materials according to any of the above embodiments can be made of various shapes according to the customized design, including implantable sensing devices, nose and ear cartilages, and blood vessels.

The polymers or bulk materials according to any of the above embodiments can have particular properties, which include, but are not limited to, the ability to support other molecules, biomolecules, small molecule drug nanoparticles, microparticles, cells, tissues, or organs as a carrier, scaffold, or matrix. Moreover, applications for bulk material ZSN, ZDN, or ZTN hydrogels according to any of the above embodiments can be made into a consumer product.

The polymers or bulk materials according to any of the above embodiments can be made into an article suited for marine use. The marine article may be selected from, for example, marine vessel hulls, marine structures, bridges, propellers, heat exchangers, periscopes, sensors, fishnets, cables, tubes/pipes, containers, membranes, and oil booms.

The polymers or bulk materials according to any of the above embodiments can be made into a biomedical product. The polymers or hydrogels according to any of the above embodiments can be made into a biomedical product selected from, for example, catheters, ear drainage tubes, feeding tubes, glaucoma drainage tubes, hydrocephalous shunts, keratoprosthesis, nerve guidance tubes, tissue adhesives, x-ray guides, artificial joints, artificial heart valves, artificial blood vessels, pacemakers, left ventricular assist devices (LVAD), artery grafts, vascular grafts, stents, intravascular stents, cardiac valves, joint replacements, blood vessel prostheses, skin repair devices, cochlear replacements, contact lenses, artificial ligaments and tendons, dental implants, and tissue scaffolds for regenerative tissue engineering.

The present disclosure is also directed to a surface coating for a substrate, wherein the surface coating contains one or more of the polymers or bulk materials (e.g., hydrogels, copolymers, or nanoparticles) according to any of the above embodiments. In some embodiments, the substrate is a consumer product. In other embodiments, the substrate is a marine product. In particular embodiments, the substrate is a marine product selected from marine vessel hulls, marine structures, bridges, propellers, heat exchangers, periscopes, sensors, fishnets, cables, tubes/pipes, containers, membranes, and oil booms. In other embodiments, the substrate is a biomedical product. In particular embodiments, the substrate is a biomedical product selected from catheters, ear drainage tubes, feeding tubes, glaucoma drainage tubes, hydrocephalous shunts, keratoprosthesis, nerve guidance tubes, tissue adhesives, x-ray guides, artificial joints, artificial heart valves, artificial blood vessels, pacemakers, left ventricular assist devices (LVAD), artery grafts, vascular grafts, stents, intravascular stents, cardiac valves, joint replacements, blood vessel prostheses, skin repair devices, cochlear replacements, contact lenses, artificial ligaments and tendons, dental implants, and tissue scaffolds for regenerative tissue engineering. In some embodiments, the substrate has one or more particular properties, which include, but are not limited to, ability to support other molecules, biomolecules, small molecule drugs nanoparticles, microparticles, cells, tissues, or organs as a carrier, scaffold, or matrix.

In yet another aspect, the present disclosure is directed to a method of producing a carboxybetaine (CB) monomer of the formula

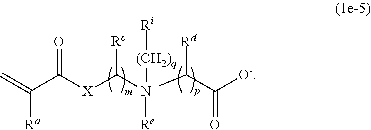

The method comprises the following steps:
(i) producing an intermediate (Int-1) by the following reaction scheme:

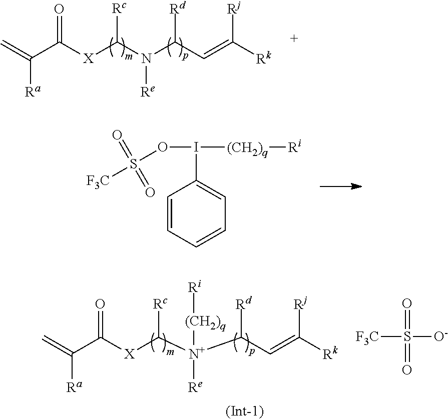

wherein:
R^{a} is H or an alkyl group containing 1-3 carbon atoms; X is O or NR^{b}, wherein R^{b} is H or an alkyl group containing 1-3 carbon atoms; m is an integer of precisely or at least 1; p is an integer of precisely or at least 1; R^{c} and R^{d} are independently selected from hydrogen atom, hydrocarbon group, and fluorinated hydrocarbon group containing 1-12 carbon atoms and at least one fluorine atom; R^{e} is selected from R^{a}; q is an integer of precisely or at least 1; Rⁱ is a fluorinated hydrocarbon group containing 1-12 carbon atoms and at least one fluorine atom; and R^{j} and R^{k} are independently selected from hydrogen atom, alkyl group containing 1-3 carbon atoms, silyl groups, and stannyl groups; wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom;
and
(ii) oxidatively cleaving the intermediate (Int-1) to produce the carboxybetaine monomer of the formula (1e-5).

In yet another aspect, the present disclosure is directed to a method of producing a trimethylammonium oxide (TMAO) monomer of the formula

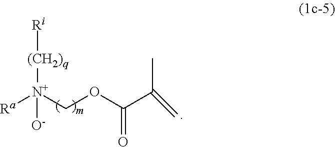

The method comprises the following steps:
(i) producing an intermediate (Int-2) by the following reaction scheme:

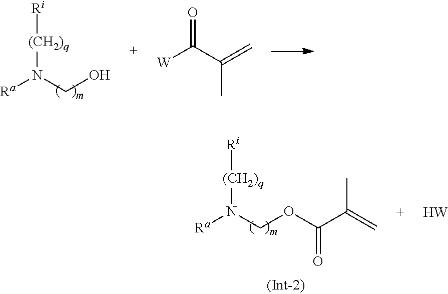

wherein: R^{a} is H or an alkyl group containing 1-3 carbon atoms; m is an integer of precisely or at least 1; q is an integer of precisely or at least 1; Rⁱ is a fluorinated hydrocarbon group containing 1-12 carbon atoms and at least one fluorine atom; and W is Cl or Br; and
(ii) oxidizing the intermediate (Int-2) to produce the TMAO monomer of the formula (1c-5).

### BRIEF DESCRIPTION OF THE FIGURES

FIG. 1. Ellipsometry of SAM on gold substrate.

FIG. 2. Ellipsometry of polymer on gold substrate.

FIG. 3. GPC chromatogram of cleaved polymer.

### DETAILED DESCRIPTION

In one aspect, the present disclosure is directed to zwitterionic monomer compositions that are fluorinated. The zwitterionic monomer compositions are capable of being polymerized into zwitterionic polymers. As used herein, the term “zwitterionic monomer” refers to a polymerizable molecule containing at least one zwitterionic moiety, wherein a zwitterionic moiety contains both negative and positively charged groups. In typical embodiments, the zwitterionic monomer compositions contain a vinyl (olefin) group attached to a zwitterionic portion, wherein the vinyl group is polymerizable by vinyl addition as well known in the art to produce a polymer containing zwitterionic pendant groups and a polyvinyl backbone. In the zwitterionic monomer, the zwitterionic moiety may alternatively be attached to polymerizable groups other than vinyl or methacryl groups, such as those polymerizable groups that can react to form backbones containing siloxane bonds, ester bonds, urethane bonds, urea bonds, and carbonate bonds (the resulting polymer may thus be, respectively, e.g., a zwitterionic polysiloxane, polyester, polyurethane, polyurea, or polycarbonate). Moreover, the zwitterionic monomer may, in some embodiments, contain a zwitterionic moiety that functions as a polymerizable group, which results in a zwitterionic polymer containing a backbone that is zwitterionic.

In some embodiments, the monomer composition is within the scope of the following general formula:

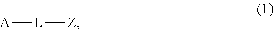

In Formula (I), A is a polymerizable group (e.g., olefin, siloxane, diacid, diester, diol, acid-hydroxy, isocyanate, or diisocyanate); L is a bond or linking portion; and Z is a zwitterionic moiety or a zwitterionic precursor moiety containing a protecting group capable of deprotection to form a charged group. For purposes of the invention, at least one hydrogen atom in A, L, and/or Z in Formula (1) is substituted by a fluorinated hydrocarbon group or fluorine atom, wherein the fluorinated hydrocarbon group contains 1-12 carbon atoms and precisely or at least one, two, three, four, five, or six fluorine atoms. The fluorinated hydrocarbon group may or may not include one or more oxygen or nitrogen atoms. The fluorinated hydrocarbon group may be partially fluorinated or fully fluorinated (perfluorinated). Some examples of fluorinated hydrocarbon groups include CF₃, CH₂CF₃, (CF₂)ᵣCF₃, CH(CF₃)₂, and CF(CF₃)₂, wherein r is, e.g., 0, 1, 2, 3, 4, 5, 6, 7, 8, 9, 10, 11, or 12. In some embodiments, for any of the formulas in this disclosure, the fluorinated hydrocarbon group is composed of only carbon, hydrogen, and fluorine atoms or composed of only carbon and fluorine atoms. In some embodiments, the fluorinated hydrocarbon group is bound to an ammonium nitrogen. In some embodiments, for any of the formulas in this disclosure, the fluorinated hydrocarbon group does not contain an oxygen or nitrogen atom, particularly when the fluorinated hydrocarbon group is bound to an ammonium nitrogen. In particular embodiments, for any of the formulas in this disclosure, the fluorinated hydrocarbon group has the formula —(CH₂)ₓ—(RF), wherein x is precisely or at least 1, 2, 3, 4, 5, or 6, and RF is a partially or fully fluorinated hydrocarbon group.

The monomer may more particularly have the following structure:

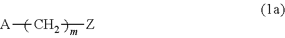

In Formula (1a), A and Z are as defined under Formula (1); and m is an integer of at least 1. In different embodiments, m is 1, 2, 3, 4, 5, 6, 7, 8, 9 10, 11, or 12, or m is within a range therein, e.g., 1-12 or 3-12, wherein the methylene groups may or may not be interrupted by a heteroatom-containing group, such as —O—, —NR′—, —C(O)O—, or —C(O)NR—). For purposes of the invention, at least one hydrogen atom in A, CH₂, and/or Z in Formula (1a) is substituted by a fluorinated hydrocarbon group or fluorine atom, wherein the fluorinated hydrocarbon group contains 1-12 carbon atoms and precisely or at least one, two, three, four, five, or six fluorine atoms. The group R′ may be a hydrogen atom or hydrocarbon group, wherein the hydrocarbon groups typically contains 1-12 carbon atoms, as described above.

In some embodiments, the monomer more particularly has the following structure:

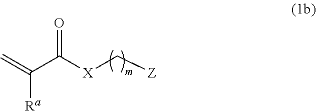

In Formula (1b), R^{a} is H or an alkyl group containing 1-3 carbon atoms (e.g., methyl, ethyl, n-propyl, or isopropyl, wherein the alkyl group optionally contains one, two, three, or more fluorine atoms); X is O or NR^{b}, wherein R^{b} is H or an alkyl group containing 1-3 carbon atoms; Z is a zwitterionic moiety or a zwitterionic precursor moiety containing a protecting group capable of deprotection to form a charged group; and m is an integer of at least 1 or as provided above. In some embodiments, at least one hydrogen atom in Formula (1b) (e.g., in olefin group, R^{a}, X, methylene group under m, or Z) is substituted by a fluorinated hydrocarbon group or fluorine atom, wherein the fluorinated hydrocarbon group contains 1-12 carbon atoms and precisely or at least one, two, three, four, five, or six fluorine atoms, as described above.

In other embodiments, the monomer more particularly has the following structure:

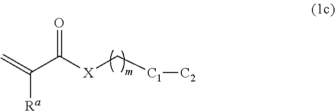

In Formula (1c), R^{a}, X, and m are as defined under Formula (1b), and C₁ and C₂ are independently selected as positively charged and negatively charged groups to form a zwitterionic moiety C₁-C₂. For purposes of the invention, at least one hydrogen atom on the olefin group, R^{a}, X, methylene group under m, C₁, and/or C₂ is substituted by a fluorinated hydrocarbon group or fluorine atom, wherein the fluorinated hydrocarbon group contains 1-12 carbon atoms and precisely or at least one, two, three, four, five, or six fluorine atoms, as described above.

The monomer of Formula (1c) may more particularly have the following structure:

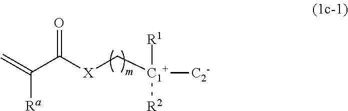

In Formula (1c-1), R^{a}, X, and m are as defined under Formula (1b), and R¹ and R² are independently selected from hydrocarbon groups containing 1-12 carbon atoms, wherein at least one of R¹ and R² contains at least one fluorine atom. C₁⁺ and C₂⁻ are positively charged and negatively charged atoms or groups, respectively, to form a zwitterionic moiety C₁⁺—C₂⁻. The dashed line represents an optional bond. The C₁⁺ atom or group may be or include, for example, a positively charged nitrogen atom, phosphorus atom, or sulfur atom. Any one or more of R^{a}, X, and methylene group under m is optionally substituted by fluorine or a fluorinated hydrocarbon group, wherein the fluorinated hydrocarbon group contains 1-12 carbon atoms and precisely or at least one, two, three, four, five, or six fluorine atoms, as described above.

The monomer of Formula (1c) may more particularly have the following structure:

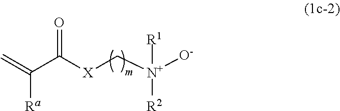

In Formula (1c-2), R^{a}, X, and m are as defined under Formula (1b), and R¹ and R² are independently selected from R^{a} or hydrocarbon groups containing 1-12 carbon atoms, wherein R¹ and/or R² (or at least one) optionally contains at least one fluorine atom, as defined earlier above. For purposes of the invention, at least one hydrogen atom on R^{a}, X, methylene group under m, R¹, and/or R² is substituted by a fluorinated hydrocarbon group or fluorine atom, wherein the fluorinated hydrocarbon group contains 1-12 carbon atoms and precisely or at least one, two, three, four, five, or six fluorine atoms, as described above.

The monomer of Formula (1c) may more particularly have the following structure:

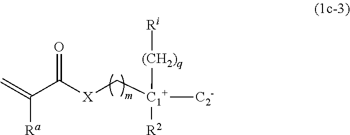

In Formula (1c-3), R^{a}, X, and m are as defined under Formula (1b), and R² is selected from R^{a} or hydrocarbon groups containing 1-12 carbon atoms and optionally containing one ore more fluorine atoms, as defined earlier above. C₁⁺ and C₂⁻ are positively charged and negatively charged atoms or groups, respectively, to form a zwitterionic moiety C₁⁺-C₂⁻. The dashed line represents an optional bond. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, and methylene group under m is/are optionally substituted by fluorine or a fluorinated hydrocarbon group, wherein the fluorinated hydrocarbon group contains 1-12 carbon atoms and at least one fluorine atom.

The monomer of Formula (1c-3) may more particularly have the following structure:

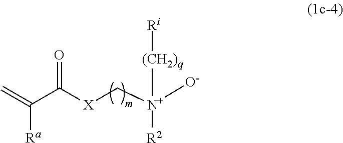

In Formula (1c-4), R^{a}, X, and m are as defined under Formula (1b), and R² is selected from R^{a} or hydrocarbon groups containing 1-12 carbon atoms and optionally containing one ore more fluorine atoms, as defined earlier above. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, and/or R² is/are optionally substituted by fluorine or a fluorinated hydrocarbon group, wherein the fluorinated hydrocarbon group contains 1-12 carbon atoms and at least one fluorine atom.

Alternatively, Z may contain a positively charged group indirectly bound to a negatively charged group via a linker, such as in the following structure:

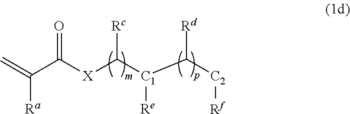

In Formula (1d), R^{a}, X, and m are as defined under Formula (1b). The variable p is an integer of at least 1. The variable p may be, for example, 1, 2, 3, 4, 5, or 6, or an integer within a range bounded by any two of the foregoing values. The variables R^{c}, R^{d}, R^{e}, and R^{f} are independently selected from hydrogen atom, hydrocarbon group, fluorine atom, and fluorinated hydrocarbon group containing 1-12 carbon atoms and precisely or at least one, two, three, four, five, or six fluorine atoms, wherein at least one of R^{c}, R^{d}, R^{e}, and R^{f} is said fluorinated hydrocarbon group or fluorine atom. Notably, R^{c} and R^{d} are each independently present twice for each methylene linkage. One or both of R^{e} and R^{f} are alternatively selected from positive and negative charges. The variables C₁ and C₂ are independently selected as positively charged and negatively charged groups (moieties) to form a zwitterionic moiety. Some examples of positively charged moieties include ammonium (—NR^{a}₂⁺—) phosphonium (—PR^{a}₂⁺—), and sulfonium moieties. Some examples of negatively charged moieties include terminal oxide (—O—), carboxylate (—C(O)O—), phosphate (—OPO₃⁻), phosphonate (—PO₃⁻), sulfate (—OSO₃⁻), and sulfonate (—SO₃⁻). In some embodiments, C₁ is neutral and R^{e} is a protecting group capable of deprotection to form a charged group, or C₂ is neutral and R^{f} is a protecting group capable of deprotection to form a charged group. The protecting group may be, for example, a bulky alkyl group (e.g., t-butyl) forming an ester with a carboxylate. Protecting groups may be removed by standard chemical means to produce a charged group to form a zwitterion. Thus, a monomer in which C₁ or C₂ contains a protecting group can be considered a zwitterionic monomer precursor. In some embodiments, C₁ or C₂ is a neutral amine group, which can be reacted with an amine-reactive compound, such as an alkyl halide, to result in a quaternary ammonium group. Thus, a monomer in which C₁ or C₂ is a neutral amine can also be considered a zwitterionic monomer precursor. In some embodiments, any of R^{c}, R^{d}, R^{e}, and R^{f} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{d} is a partially or fluorinated hydrocarbon group containing 1-12, 1-10, 1-8, or 1-6 carbon atoms, or more particularly, has the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In more particular embodiments, the olefin monomer of Formula (1d) has the following structure:

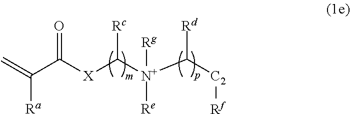

In Formula (1e), R^{a}, X, m, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c}, R^{d}, R^{e}, R^{f}, and R^{g} are independently selected from hydrogen atom, hydrocarbon group, fluorine atom, and fluorinated hydrocarbon groups containing 1-12 carbon atoms and precisely or at least one, two, three, four, five, or six fluorine atoms. For purposes of the invention, at least one of R^{c}, R^{d}, R^{e}, R^{f}, and R^{g} is said fluorinated hydrocarbon group or fluorine atom (except that a fluorine atom is not directly attached to nitrogen). In some embodiments, R^{f} is alternatively a negative charge. The variable C₂ is a negatively charged group. Optionally, C₂ is neutral and R^{f} is a protecting group capable of deprotection to form a charged group. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{c} or R^{d} is a partially or fluorinated hydrocarbon group containing 1-12, 1-10, 1-8, or 1-6 carbon atoms, or more particularly, has the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In some embodiments, the olefin monomer of Formula (1e) may more particularly have the following structure:

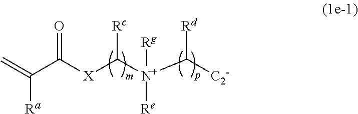

In Formula (1e-1), R^{a}, X, m, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c}, R^{d}, R^{e}, and R^{g} are independently selected from hydrogen atom, hydrocarbon group, fluorine atom, and fluorinated hydrocarbon groups containing 1-12 carbon atoms and precisely or at least one, two, three, four, five, or six fluorine atoms. For purposes of the invention, at least one of R^{c}, R^{d}, R^{e}, R^{f}, and R^{g} is said fluorinated hydrocarbon group or a fluorine atom (except that a fluorine atom is not directly attached to nitrogen). The variable C₂ is a negatively charged group, such as a carboxylate, sulfonate, sulfate, phosphonate, or phosphate group. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{c} or R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In some embodiments, the olefin monomer of Formula (1d) may more particularly have the following structure:

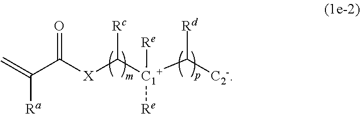

In Formula (1e-2), R^{a}, X, m, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. Each R^{e} is independently selected from hydrocarbon groups containing 1-12 carbon atoms and R^{a}, wherein one or both R^{e} may contain at least one fluorine atom. C₁⁺ and C₂⁻ are positively charged and negatively charged atoms or groups, respectively, to form a zwitterionic moiety C₁⁺-C₂⁻. The dashed line represents an optional bond. Any one or more of R^{a}, X, methylene group under m, R^{c}, and R^{d} is/are optionally substituted by fluorine or a fluorinated hydrocarbon group, wherein the fluorinated hydrocarbon group contains 1-12 carbon atoms and at least one fluorine atom. In some embodiments, only or at least R^{c} or R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In some embodiments, the olefin monomer of Formula (1e-2) may more particularly have the following structure:

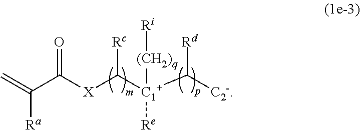

In Formula (1e-3), R^{a}, X, and m are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. Each R^{e} is independently selected from hydrocarbon groups containing 1-12 carbon atoms and R^{a}, wherein at least one R contains at least one fluorine atom. C₁⁺ and C₂⁻ are positively charged and negatively charged atoms or groups, respectively, to form a zwitterionic moiety C₁⁺-C₂⁻. The dashed line represents an optional bond. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and R^{d} is/are optionally substituted by fluorine or a fluorinated hydrocarbon group, wherein the fluorinated hydrocarbon group contains 1-12 carbon atoms and at least one fluorine atom.

In some embodiments, the olefin monomer of Formula (1e-3) may more particularly have the following structure:

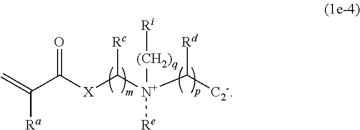

In Formula (1e-4), R^{a}, X, and m are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. R^{e} is selected from hydrocarbon groups containing 1-12 carbon atoms and R^{a}. The variable C₂⁻ is a negatively charged group, such as a carboxylate, sulfonate, sulfate, phosphonate, or phosphate group. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and/or R^{d} is/are optionally substituted by fluorine or a fluorinated hydrocarbon group, wherein the fluorinated hydrocarbon group contains 1-12 carbon atoms and at least one fluorine atom.

In some embodiments, the olefin monomer of Formula (1e-4) may more particularly have the following structure:

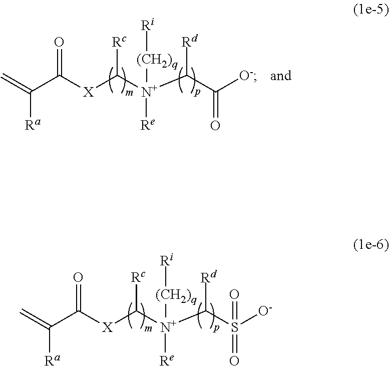

In Formulas (1e-5) and (1e-6), R^{a}, X, and m are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. R^{e} is selected from hydrocarbon groups containing 1-12 carbon atoms and R^{a}. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and/or R^{d} is/are optionally substituted by fluorine or a fluorinated hydrocarbon group, wherein the fluorinated hydrocarbon group contains 1-12 carbon atoms and at least one fluorine atom.

The olefin monomer of Formula (1e-1) may more particularly have any of the following structures:

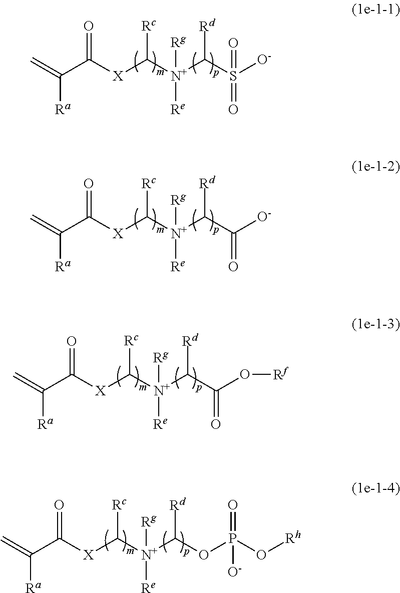

In any of the above formulas, R^{a}, X, and m are as defined above. The variable p is an integer of at least 1. The variables R^{c}, R^{d}, R^{e}, R^{f}, R^{g}, and R^{h} are selected from hydrogen atom, hydrocarbon group, fluorine atom, and fluorinated hydrocarbon groups containing 1-12 carbon atoms and precisely or at least one, two, three, four, five, or six fluorine atoms. For purposes of the invention, at least one of R^{c}, R^{d}, R^{e}, R^{f}, R^{g}, and R^{h} is said fluorinated hydrocarbon group or fluorine atom. In some embodiments, the variable R is alternatively a protecting group capable of deprotection to form a carboxylate group. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fluorinated hydrocarbon group containing 1-12, 1-10, 1-8, 1-6, 1-4, 1-3, 2-6, 2-4, or 3-6 carbon atoms. In some embodiments, only or at least R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In other embodiments of Formula (1e), the olefin monomer composition has the following structure:

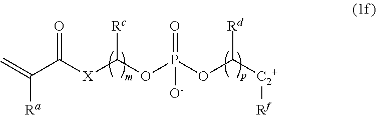

In Formula (1f), R^{a}, X, and m are as defined earlier above. The variable C₂⁺ is a positively charged group. The variable p is an integer of at least 1. The variables R^{c}, R^{d}, and R^{f} are selected from hydrogen atom, fluorine atom, hydrocarbon group, and fluorinated hydrocarbon group containing 1-12 carbon atoms and precisely or at least one, two, three, four, five, or six fluorine atoms, wherein at least one of R^{c}, R^{d}, and R^{f} is said fluorinated hydrocarbon group. In some embodiments, R^{f} is alternatively absent.

In particular embodiments of Formula (1f), the olefin monomer has the following structure:

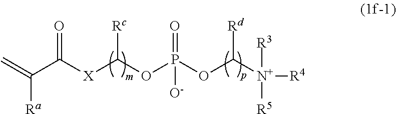

In Formula (1f-1), R^{a}, X, and m are as defined above, and p is an integer of at least 1. The variables R^{c}, R^{d}, R³, R⁴, and R⁵ are selected from hydrogen atom, hydrocarbon group, fluorine atom, and fluorinated hydrocarbon groups containing 1-12 carbon atoms and precisely or at least one, two, three, four, five, or six fluorine atoms, wherein at least one of R^{c}, R^{d}, R³, R⁴, and R⁵ is said fluorinated hydrocarbon group or fluorine atom.

The present disclosure is also directed to polymers of any of the zwitterionic monomers described above, such as those in Formulas (1), (1a), (1b), (1c), (1c-1), (1d), (1e), (1e-1), (1e-1-1), (1e-1-2), (1e-1-3), (1e-1-4), (1f), and (1f-1). As used herein, the term “zwitterionic polymer” refers to a polymer containing zwitterionic moieties, wherein a zwitterionic moiety contains both negative and positively charged groups covalently attached to the polymer. In one set of embodiments, the zwitterionic polymer is produced by polymerization of a polymerizable zwitterionic monomer or a monomer containing a precursor for a zwitterionic group. Zwitterionic monomers are electrically neutral monomers that include equal numbers of positive and negative charges per monomer. In another set of embodiments, the zwitterionic polymer is produced by polymerization of equal numbers of monomers containing negatively charged groups and monomers containing positively charged groups. In some cases, the polymer is produced solely from zwitterionic monomers or equal numbers of positively and negatively charged monomers, in which case the zwitterionic polymer has 100 mole percent (100 mol %) zwitterionic moieties. In other cases, the polymerization process may include uncharged monomers, which provides a zwitterionic copolymer having less than 100 mole percent zwitterionic moieties. For example, when a polymerizable zwitterionic monomer and polymerizable comonomer are present in equal proportions in the polymerization mixture, the product is a zwitterionic polymer having 50 mol % zwitterionic moieties. The uncharged monomer may be, for example, ethylene, propylene, styrene, methyl acrylate, methyl methacrylate, 2-hydroxyethyl acrylate, 2-ethoxyethyl acrylate, 2-(2-ethoxyethoxy)ethyl acrylate, acrylamide, vinyl alcohol, or acrylonitrile.

The polymer is typically a homopolymer of any of the foregoing zwitterionic monomers. In some embodiments, the polymer may be a copolymer (e.g., block, random, or alternating) containing any two or more of the above zwitterionic monomers, in the absence or presence of a non-zwitterionic monomer. In other embodiments, the polymer may be a copolymer containing one or two of any of the above zwitterionic monomers and one or more non-zwitterionic monomers (e.g., styrene, butadiene, or a fluorinated vinyl molecule).

The polymers may be, for example, any of the polymers of the Formulas (I), (Ia), (Ib), (Ic), (Ic-1), (Id), (Ie), (Ie-1), (Ie-1-1), (Ie-1-2), (Ie-1-3), (Ie-1-4), (If), and (If-1), as depicted in the claims. The polymers may be homopolymers of any of the monomers described above, or the polymers may be copolymers containing any of the zwitterionic monomers described above copolymerized with a non-zwitterionic monomer (e.g., vinyl alcohol, acrylate, methacrylate, trifluoroethylene, tetrafluoroethylene, styrene, or butadiene, or fluorinated versions of any one of these).

The zwitterionic polymer may have the following formula:

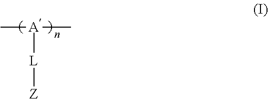

In Formula (I), A′ is a backbone of the polymer formed by polymerization of A. The backbone (A) can be any polymeric backbones known in the art, as described above and which may include linear, branched, and cyclic backbone structures. The backbone may be, for example, olefinic (e.g., polyvinyl or polymethacryl), polysiloxane, polyester, polyurethane, polyurea, polycarbonate, polypeptide, polyimide, polyphosphazene, polyepoxy, phenolic polymer, polysulfone, or a polysulfide. In the case of vinyl monomers, as largely described above, the backbone has a polyethylene (polyvinyl) structure, which may or may not be substituted with one or more fluorine atoms. The variable L is a bond or a linking portion, as described above, which may or may not be substituted with one or more fluorine atoms. The linker (L) can be any of the linkers commonly included in pendant groups of polymers, e.g., linear or branched alkyl linkers or cyclic linkers, any of which may contain precisely or at least 1, 2, 3, 4, 5, or 6 carbon atoms and optionally containing one or more heteroatoms (typically selected from oxygen and nitrogen atoms). Z is a zwitterionic moiety or a zwitterionic precursor moiety containing a protecting group capable of deprotection to form a charged group, as described above. The subscript n is typically an integer of at least 2, 3, 4, 5, 6, 7, 8, 9, 10, 20, 30, 40, 50, 100, 200, 300, 400, 500, 1000, 5000, 10,000, 50,000, or 100,000 units, or a number of units within a range bounded by any two of the foregoing values. For purposes of the invention, at least one hydrogen atom in A′, L, and/or Z in Formula (I) is substituted by a fluorinated hydrocarbon group or fluorine atom, wherein the fluorinated hydrocarbon group contains 1-12 carbon atoms and precisely or at least one, two, three, four, five, or six fluorine atoms, as described above. As indicated earlier, Formula (I) includes copolymers of Formula (I), unless otherwise specified. In particular embodiments, Z is selected from the group consisting of carboxybetaine, sulfobetaine, phosphobetaine, and trialkylamine-N-oxide zwitterionic moieties.

In some embodiments of Formula (I), the zwitterionic polymer has the following formula:

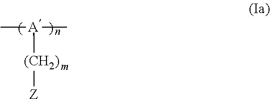

wherein A′ is a backbone, as described above; Z is a zwitterionic moiety or a zwitterionic precursor moiety, as described above; n is an integer of at least 2, and m is an integer of at least 1, as described earlier above. In particular embodiments, Z is selected from the group consisting of carboxybetaine, sulfobetaine, phosphobetaine, and trialkylamine-N-oxide zwitterionic moieties.

In other embodiments of Formula (I), the zwitterionic polymer has the following formula:

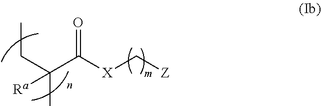

In Formula (Ib), R^{a} is H or an alkyl group containing 1-3 carbon atoms, as described earlier above. Some examples of alkyl groups containing 1-3 carbon atoms include methyl, ethyl, n-propyl, and isopropyl. In some embodiments, R^{a} is H or methyl. The variable X is O or NR^{b}, wherein R^{b} is H or an alkyl group containing 1-3 carbon atoms, as described earlier above. In some embodiments, R^{b} is H or methyl. The variable Z is a zwitterionic moiety or a zwitterionic precursor moiety containing a protecting group capable of deprotection to form a charged group, as described earlier above. In particular embodiments, Z is selected from the group consisting of carboxybetaine, sulfobetaine, phosphobetaine, and trialkylamine-N-oxide zwitterionic moieties. The subscript n is typically an integer of at least 2, 5, or 10 (e.g., at least or greater than 10, 50, 100, 200, 300, 400, 500, 1000, 5000, 10,000, 50,000, or 100,000 units, or a number of units within a range bounded by any two of the foregoing values). The subscript m is an integer of at least 1, such as a value of precisely or at least 1, 2, 3, 4, 5, 6, 7, 8, 9, 10, 11, or 12, or a value within a range bounded by any two of the foregoing values (e.g., 1-12, 1-10, 1-6, 1-4, 1-3, 2-4, or 2-3). For purposes of the invention, at least one hydrogen atom in Formula (Ib) is substituted by a fluorinated hydrocarbon group, wherein the fluorinated hydrocarbon group contains 1-12 carbon atoms and precisely or at least one, two, three, four, five, or six fluorine atoms.

In some embodiments, the group Z represents a single zwitterionic group containing a positive and negative charge in the same Z group. The group Z may be selected from, for example, carboxybetaine, sulfobetaine, phosphobetaine, and trialkylamine-N-oxide zwitterionic moieties. The resulting polymer may be, for example, a poly(carboxybetaine), poly(sulfobetaine), poly(phosphobetaine), and poly(trialkylamine-N-oxide), as well known in the art. In other embodiments, a certain number of the Z groups are positively charged and an equal number of Z groups are negatively charged to result in Z zwitterionic pairs (i.e., Z⁺Z⁻ zwitterionic pairs).

Notably, although the formulas and sub-formulas provided throughout this disclosure may appear to depict zwitterionic homopolymers (100 mol % zwitterionic moieties), the formulas and sub-formulas thereof include the possibility that one or more non-zwitterionic or uncharged monomer units is situated (inserted) between zwitterionic monomeric units depicted in any of these formulas, thereby resulting in a copolymer. If zwitterionic monomeric units (such as any of those described above, i.e., where n=1) are labeled as A units, and non-zwitterionic or uncharged monomeric units are labeled as B units, the copolymer can have any of the known copolymer arrangements, including alternating (e.g., A-B-A-B), block (e.g., A-A-A-A-B-B-B-B), or random (e.g., A-B-B-A-B-A-A-B-A-B-B). The zwitterionic polymer may also include more than one type of non-zwitterionic or uncharged monomer unit, such as in the structures A-B-C-A-B-C (repeating), A-A-A-B-B-B-C-C-C (block), or A-C-B-B-C-A-B-C-A-C-A-B-C-A-B-C-B (random), wherein B and C represent non-zwitterionic or uncharged monomeric units. The copolymers may also include more than one type of zwitterionic units, in which case A can represent two or more types of zwitterionic monomer units.

In some embodiments, Z contains a positively charged group directly bound to a negatively charged group to result in the following polymer structure:

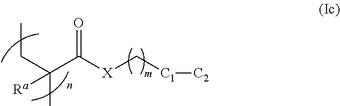

In Formula (Ic), R^{a}, X, n, and m are as defined earlier above, and C₁ and C₂ are independently selected as positively charged and negatively charged groups to form a zwitterionic moiety C₁-C₂. Some examples of positively charged moieties include ammonium (—NR^{a}₂⁺—) and phosphonium (—PR^{a}₂⁺—) moieties. Some examples of negatively charged moieties include terminal oxide (—O⁻), carboxylate (—C(O)O—), phosphate (—OPO₃⁻), phosphonate (—PO₃⁻), sulfate (—OSO₃⁻), and sulfonate (—SO₃⁻). In some embodiments, C₁ is positively charged and C₂ is negatively charged. For example, C₁ may be an ammonium moiety and C₂ may be oxide, which together results in an ammonium N-oxide (—NR^{a}₂⁺—O⁻) zwitterionic group. As the ammonium moiety is also attached to a carbon atom of the polymer, the ammonium N-oxide zwitterionic group is also herein referred to as a trialkylamine-N-oxide group. In specific embodiments, m has a value of 1, 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl. For purposes of the invention, at least one hydrogen atom on R^{a}, X, methylene group under m, C₁, and/or C₂ is substituted by a fluorinated hydrocarbon group or fluorine atom, wherein the fluorinated hydrocarbon group contains 1-12 carbon atoms and precisely or at least one, two, three, four, five, or six fluorine atoms. In other embodiments, C₁ is negatively charged and C₂ is positively charged.

In some embodiments, the polymer of Formula (Ic) more particularly has the following structure:

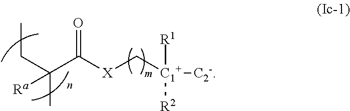

In Formula (Ic-1), R^{a}, X, and m are as defined under Formula (1b), and R¹ and R² are independently selected from hydrocarbon groups containing 1-12 carbon atoms, wherein at least one of R¹ and R² contains at least one fluorine atom. C₁⁺ and C₂⁻ are positively charged and negatively charged atoms or groups, respectively, to form a zwitterionic moiety C₁⁺-C₂⁻. The dashed line represents an optional bond. The C₁⁺ atom or group may be or include, for example, a positively charged nitrogen atom, phosphorus atom, or sulfur atom. Any one or more of R^{a}, X, and methylene group under m is optionally substituted by fluorine or a fluorinated hydrocarbon group, wherein the fluorinated hydrocarbon group contains 1-12 carbon atoms and precisely or at least one, two, three, four, five, or six fluorine atoms, as described above.

In particular embodiments of Formula (Ic), C₁ is an ammonium moiety and C₂ is oxide, which together results in an ammonium N-oxide (—NR^{a}₂⁺—O⁻) zwitterionic group. The resulting polymer is a poly(trialkylammonium oxide), i.e., pTMAO, and may have the following structure:

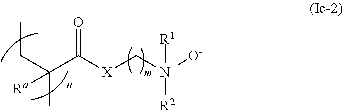

In Formula (Ic-2), R^{a}, X, n, and m are as defined earlier above, and R¹ and R² are independently selected from R^{a}, as defined earlier above. In some embodiments, R¹ and R² are both alkyl, or more particularly, both methyl. In specific embodiments, m has a value of 1, 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl. For purposes of the invention, at least one hydrogen atom on R^{a}, X, methylene group under m, R¹, and/or R² is substituted by a fluorinated hydrocarbon group or fluorine atom, wherein the fluorinated hydrocarbon group contains 1-12 carbon atoms and precisely or at least one, two, three, four, five, or six fluorine atoms.

In some embodiments, the polymer of Formula (Ic) more particularly has the following structure:

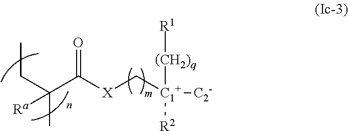

In Formula (Ic-3), R^{a}, X, and m are as defined under Formula (1b), and R² is selected from R^{a} or hydrocarbon groups containing 1-12 carbon atoms and optionally containing one ore more fluorine atoms, as defined earlier above. C₁⁺ and C₂⁻ are positively charged and negatively charged atoms or groups, respectively, to form a zwitterionic moiety C₁⁺-C₂⁻. The dashed line represents an optional bond. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, and methylene group under m is/are optionally substituted by fluorine or a fluorinated hydrocarbon group, wherein the fluorinated hydrocarbon group contains 1-12 carbon atoms and at least one fluorine atom.

In some embodiments, the polymer of Formula (Ic-3) more particularly has the following structure:

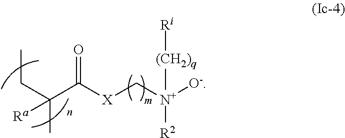

In Formula (Ic-4), R^{a}, X, and m are as defined under Formula (1b), and R² is selected from R^{a} or hydrocarbon groups containing 1-12 carbon atoms and optionally containing one ore more fluorine atoms, as defined earlier above. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, and/or R² is/are optionally substituted by fluorine or a fluorinated hydrocarbon group, wherein the fluorinated hydrocarbon group contains 1-12 carbon atoms and at least one fluorine atom.

In some embodiments of Formula (I), Z contains a positively charged group indirectly bound to a negatively charged group via a linker. The resulting polymer may then have the following structure:

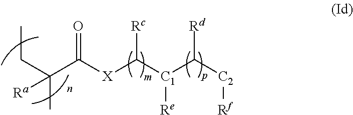

In Formula (Id), R^{a}, X, n, m, R^{c}, R^{d}, R^{e}, and R^{f} are as defined earlier above. The subscript p is an integer of at least 1, such as a value of precisely or at least 1, 2, 3, 4, 5, 6, 7, 8, 9, 10, 11, or 12, or a value within a range bounded by any two of the foregoing values (e.g., 1-12, 1-10, 1-6, 1-4, 1-3, 2-4, or 2-3). The variables R^{c}, R^{d}, R^{e}, and R^{f} are selected from hydrogen atom, hydrocarbon group, and fluorinated hydrocarbon group containing 1-12 carbon atoms and precisely or at least one, two, three, four, five, or six fluorine atoms, wherein at least one of R^{c}, R^{d}, R^{e}, and R^{f} is said fluorinated hydrocarbon group. Notably, R^{c} and R^{d} are each independently present twice for each methylene linkage. One or both of R^{e} and R^{f} may alternatively be selected from positive and negative charges. The variables C₁ and C₂ are independently selected as positively charged and negatively charged groups to form a zwitterionic moiety. Some examples of positively charged moieties include ammonium (—NR^{a}₂⁺—), phosphonium (—PR^{a}₂⁺—), and sulfonium moieties. Some examples of negatively charged moieties include terminal oxide (—O⁻), carboxylate (—C(O)O—), phosphate (—OPO₃⁻), phosphonate (—PO₃⁻), sulfate (—OSO₃⁻), and sulfonate (—SO₃⁻). In some embodiments, C₁ is positively charged and C₂ is negatively charged. For example, C₁ may be an ammonium or phosphonium moiety and C₂ may be carboxylate, sulfate, sulfonate, phosphate, or phosphonate moiety. In other embodiments, C₁ is negatively charged and C₂ is positively charged. For example, C₁ may be a phosphate, phosphonate, sulfate, or sulfonate moiety and C₂ may be an ammonium or phosphonium moiety. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In other separate or further specific embodiments, X is 0 or NR^{b}, wherein R^{b} is H or an alkyl group containing 1-3 carbon atoms, or R^{b} is H or methyl. In some embodiments, C₁ is optionally neutral and R^{e} is a protecting group capable of deprotection to form a charged group, or C₂ is optionally neutral and R^{f} is a protecting group capable of deprotection to form a charged group. In some embodiments, any of R^{c}, R^{d}, R^{e}, and R^{f} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{d} is a partially or fluorinated hydrocarbon group containing 1-12, 1-10, 1-8, or 1-6 carbon atoms, or more particularly, has the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In particular embodiments of Formula (Id), the polymer has the following structure:

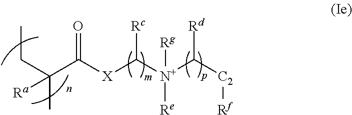

In Formula (Ie), R^{a}, X, n, m, p, R^{c}, R^{d}, R^{e}, R^{f}, and R^{g} are as defined earlier above. The variables R^{c}, R^{d}, R^{e}, R^{f}, and R^{g} are independently selected from hydrogen atom, hydrocarbon group, and fluorinated hydrocarbon group containing 1-12 carbon atoms and precisely or at least one, two, three, four, five, or six fluorine atoms, wherein at least one of R^{c}, R^{d}, R^{e}, R^{f}, and R^{g} is said fluorinated hydrocarbon group or fluorine atom (except that a fluorine atom is not directly attached to nitrogen). In some embodiments, R^{f} is alternatively a negative charge. The variable C₂ is a negatively charged group. Optionally, C₂ is neutral and R^{f} is a protecting group capable of deprotection to form a charged group. The variable p is an integer of at least 1, as described earlier above. The variable p may be, for example, 1, 2, 3, 4, 5, or 6, or an integer within a range bounded by any two of the foregoing values. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{c} or R^{d} is a partially or fluorinated hydrocarbon group containing 1-12, 1-10, 1-8, or 1-6 carbon atoms, or more particularly, has the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In particular embodiments of Formula (1e), C₁ is an ammonium moiety and C₂ is a negatively charged moiety, such as a carboxylate, sulfate, sulfonate, phosphate, or phosphonate moiety, which together results in a spaced zwitterionic group. The resulting polymer may have the following structure:

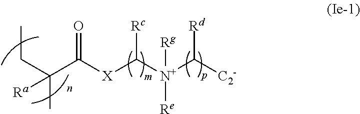

In Formula (Ie-1), R^{a}, X, n, m, p, R^{c}, R^{d}, R^{e}, and R^{g} are as defined earlier above. The variables R^{c}, R^{d}, R^{e}, and R^{g} are selected from hydrogen atom and fluorinated hydrocarbon group containing 1-12 carbon atoms and precisely or at least one, two, three, four, five, or six fluorine atoms, wherein at least one of R^{c}, R^{d}, R, R^{f}, and R^{g} is said fluorinated hydrocarbon group or a fluorine atom (except that a fluorine atom is not directly attached to nitrogen). The variable p is an integer of at least 1. The variable p may be, for example, 1, 2, 3, 4, 5, or 6, or an integer within a range bounded by any two of the foregoing values. The variable C₂ is a negatively charged group (e.g., carboxylate, sulfonate, phosphonate, phosphinate, sulfate, or phosphate). In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl. In embodiments where C₂ is a carboxylate moiety, the polymer of Formula (1e-1) can generally be referred to as a poly(carboxybetaine). In embodiments where C₂⁻ is a sulfonate moiety, the polymer of Formula (1e-1) can generally be referred to as a poly(sulfobetaine). In embodiments where C₂⁻ is a phosphate moiety, the polymer of Formula (1e-1) can generally be referred to as a poly(phosphobetaine). In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fully fluorinated hydrocarbon group containing 1-12, 1-10, 1-8, or 1-6 carbon atoms. In some embodiments, only or at least R^{c} or R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In some embodiments, the polymers of Formula (Ie-1) have the following structure:

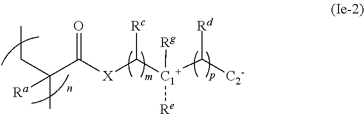

In Formula (Ie-2), R^{a}, X, m, p, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. Each R^{e} is independently selected from hydrocarbon groups containing 1-12 carbon atoms and R^{a}, wherein one or both R^{e} may contain at least one fluorine atom. C₁⁺ and C₂⁻ are positively charged and negatively charged atoms or groups, respectively, to form a zwitterionic moiety C₁⁺-C₂⁻. The dashed line represents an optional bond. Any one or more of R^{a}, X, methylene group under m, R^{c}, and R^{d} is/are optionally substituted by fluorine or a fluorinated hydrocarbon group, wherein the fluorinated hydrocarbon group contains 1-12 carbon atoms and at least one fluorine atom. In some embodiments, only or at least R^{c} or R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

In some embodiments, the polymers of Formula (Ie-2) have the following structure:

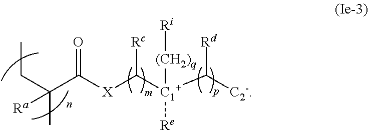

In Formula (Ie-3), R^{a}, X, m, p, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. Each R^{e} is independently selected from hydrocarbon groups containing 1-12 carbon atoms and R^{a}, wherein at least one R^{e} contains at least one fluorine atom. C₁⁺ and C₂⁻ are positively charged and negatively charged atoms or groups, respectively, to form a zwitterionic moiety C₁⁺-C₂⁻. The dashed line represents an optional bond. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and R^{d} is/are optionally substituted by fluorine or a fluorinated hydrocarbon group, wherein the fluorinated hydrocarbon group contains 1-12 carbon atoms and at least one fluorine atom.

In some embodiments, the polymers of Formula (Ie-3) have the following structure:

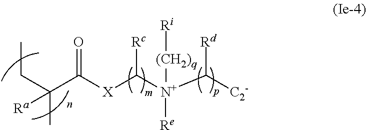

In Formula (Ie-4), R^{a}, X, m, p, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. R^{e} is selected from hydrocarbon groups containing 1-12 carbon atoms and R^{a}. The variable C₂⁻ is a negatively charged group, such as a carboxylate, sulfonate, sulfate, phosphonate, or phosphate group. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and/or R^{d} is/are optionally substituted by fluorine or a fluorinated hydrocarbon group, wherein the fluorinated hydrocarbon group contains 1-12 carbon atoms and at least one fluorine atom.

In some embodiments, the polymer of Formula (Ie-4) may more particularly have any of the following structures:

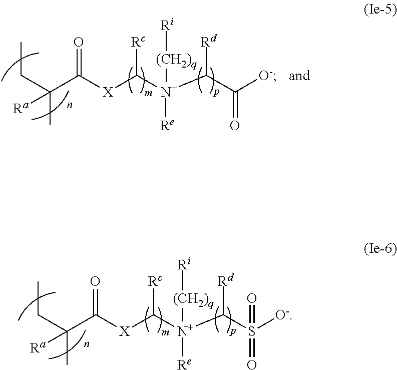

In Formulas (1e-5) and (1e-6), R^{a}, X, m, p, R^{c}, R^{d}, and R^{e} are as defined earlier above. The variable p is an integer of at least 1. The variable p may be, for example, 1, 2, 3, 4, 5, or 6, or an integer within a range bounded by any two of the foregoing values. The variables R^{c} and R^{d} are independently selected from hydrogen atom, hydrocarbon group, fluorine atom, and fluorinated hydrocarbon groups containing 1-12 carbon atoms and precisely or at least one, two, three, four, five, or six fluorine atoms. R^{e} is selected from hydrocarbon groups containing 1-12 carbon atoms and R^{a}. The subscript q is an integer of precisely or at least 1. In different embodiments, q is precisely or at least 1, 2, 3, or 4, or q is within a range bounded by any two of the foregoing values. Rⁱ is a fluorinated hydrocarbon group containing 1-12, 1-10, 1-8, 1-6, 1-4, 1-3, 2-6, 2-4, or 3-6 carbon atoms and precisely or at least one, two, three, or more fluorine atoms, as described above. Typically, at least the carbon in Rⁱ attaching to (CH₂)_{q} contains at least one F atom. Any one or more of R^{a}, X, methylene group under m, R^{c}, and/or R^{d} is/are optionally substituted by fluorine or a fluorinated hydrocarbon group, wherein the fluorinated hydrocarbon group contains 1-12 carbon atoms and at least one fluorine atom.

In particular embodiments of Formula (Ie-1), the polymers may have any of the following structures:

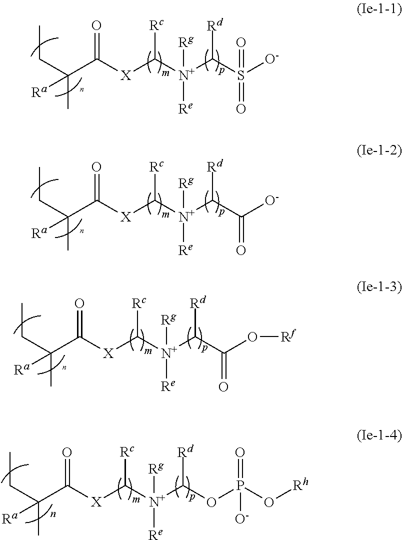

In the above formulas, R^{a}, X, n, m, p, R^{c}, R^{d}, R^{e}, R^{f}, R^{g}, and R^{h} are as defined earlier above. The variables R^{c}, R^{d}, R^{e}, R^{f}, R^{g}, and R^{h} are independently selected from hydrogen atom and fluorinated hydrocarbon group containing 1-12 carbon atoms and precisely or at least one, two, three, four, five, or six fluorine atoms, wherein at least one of R^{c}, R^{d}, R, R, R^{g}, and R^{h} is said fluorinated hydrocarbon group. The variable p is an integer of at least 1, as described earlier above. In some embodiments, R^{g} and/or R^{e} are selected from fluorinated hydrocarbon groups that do not contain an ether linkage or that do not contain oxygen and/or nitrogen atoms. In some embodiments, R^{g} and/or R^{e} has the formula —(CH₂)ₓ—(RF), wherein x is precisely or at least 1, 2, 3, 4, 5, or 6, and RF is a partially or fluorinated hydrocarbon group containing 1-12, 1-10, 1-8, 1-6, 1-4, 1-3, 2-6, 2-4, or 3-6 carbon atoms. In some embodiments, only or at least R^{d} is a partially or fluorinated hydrocarbon group containing 1-12, 1-10, 1-8, or 1-6 carbon atoms, or more particularly, the formula —(CH₂)ₓ—(RF), wherein x is precisely or at least 1, 2, 3, 4, 5, or 6, and RF is a partially or fluorinated hydrocarbon group containing 1-12, 1-10, 1-8, or 1-6 carbon atoms.

Notably, any of the above zwitterionic polymers containing an ammonium group may be modified by replacing N⁺ in the structure with P⁺ to generate an equivalent number of phosphonium zwitterionic polymers. Such polymers may have the general structure:

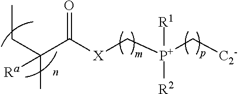

or the following more particular structures:

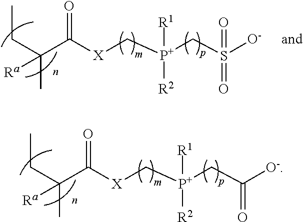

In other embodiments of Formula (Id), C₁ is a phosphate moiety and C₂ is a positively charged moiety, such as an ammonium or phosphonium moiety, which together results in a spaced zwitterionic group. The resulting polymers may have the following structure:

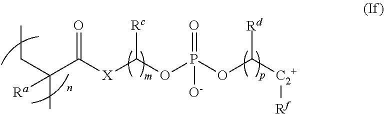

In Formula (If), R^{a}, X, n, and m are as defined earlier above. The variable C₂⁺ is a positively charged moiety that forms a zwitterionic spaced pair with the phosphate moiety in Formula (If). Some examples of positively charged moieties include ammonium (—NR^{a}₂⁺—) and phosphonium (—PR^{a}₂⁺—) moieties. The variable p is an integer of at least 1, as described earlier above. The variables R^{c}, R^{d}, and R^{f} are selected from hydrogen atom, hydrocarbon group, and fluorinated hydrocarbon group containing 1-12 carbon atoms and precisely or at least one, two, three, four, five, or six fluorine atoms, wherein at least one of R^{c}, R^{d}, and R^{f} is said fluorinated hydrocarbon group. In some embodiments, R^{f} is alternatively absent. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In other separate or further specific embodiments, X is O or NR^{b}, wherein R^{b} is H or an alkyl group containing 1-3 carbon atoms, or R^{b} is H or methyl.

In particular embodiments of Formula (If), C₂⁺ is an ammonium moiety. The resulting polymers may have the following structure:

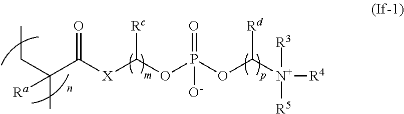

In Formula (If-1), R^{a}, X, n, and m are as defined earlier above. The variable p is an integer of at least 1, as described earlier above. The variables R^{c}, R^{d}, R³, R⁴, and R⁵ are selected from hydrogen atom and fluorinated hydrocarbon group containing 1-12 carbon atoms and precisely or at least one, two, three, four, five, or six fluorine atoms, wherein at least one of R^{c}, R^{d}, R³, R⁴, and R⁵ is said fluorinated hydrocarbon group. In separate or further specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl.

Some specific examples of zwitterionic polymers within the above formulas include:

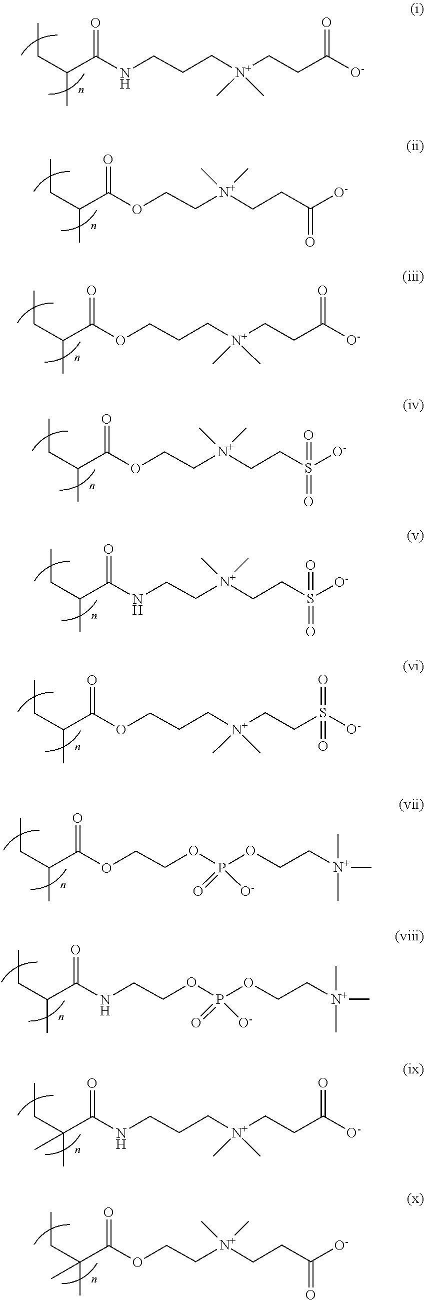

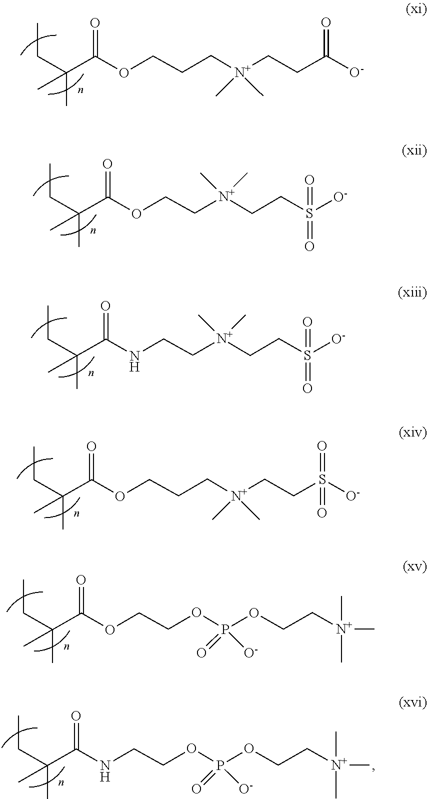

wherein at least one hydrogen atom in each structure above is substituted with a fluorine atom or fluorinated hydrocarbon group.

Other types of zwitterionic polymers within the scope of Formula (I) include:

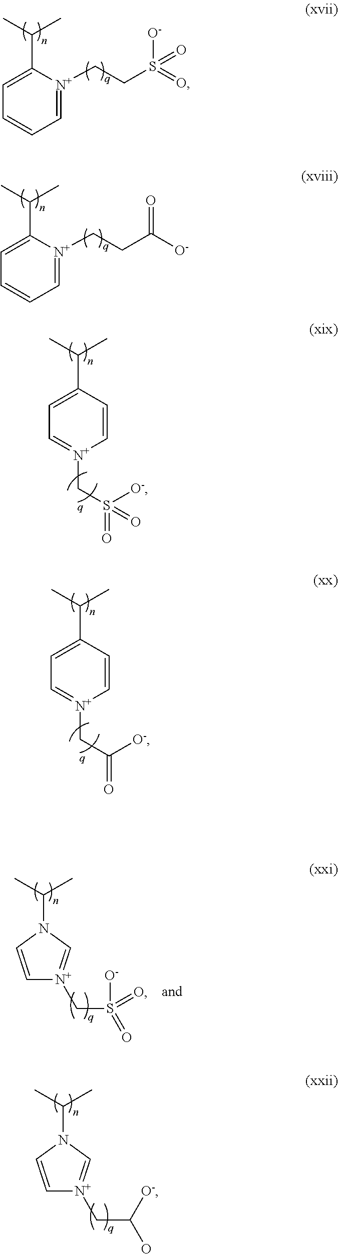

wherein q can have any of the values given above for m (e.g., at least 1), and wherein at least one hydrogen atom in each structure is substituted with a fluorine atom or fluorinated hydrocarbon group.

The zwitterionic polymers may alternatively be mixed-charged zwitterionic copolymers. Such polymers may be conveniently represented by the following formula:

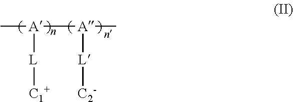

In Formula (II), A′ and A″ are backbones of different copolymer segments. L and L′ are same or different linking portions. The variable C₁⁺ is a positively charged group, and C₂⁻ is a negatively charged group, wherein C₁⁺ and C₂⁻ together form a zwitterionic system. The variables n and n′ are independently selected from integers of at least 2. For purposes of the invention, at least one hydrogen atom in A′, A″, L, L′, C₁⁺, and/or C₂⁻ in Formula (I) is substituted by a fluorinated hydrocarbon group or fluorine atom, wherein the fluorinated hydrocarbon group contains 1-12 carbon atoms and precisely or at least one, two, three, four, five, or six fluorine atoms. In some embodiments, A′ and A″ are olefinic. Notably, the polymer of Formula (II) may have any of the known copolymer arrangements, including block, alternating, periodic, and random arrangements. Moreover, although Formula (II) depicts a binary copolymer, the copolymer of Formula (II), may or may not contain one or more additional units or segments to result in a ternary or quaternary copolymer.

The present disclosure is also directed to bulk materials containing any one, two, or three of the zwitterionic compositions described above. In one instance, the bulk material is a copolymer of any of the fluorinated zwitterionic monomers described above and non-zwitterionic monomers to result in a copolymer hybrid composition. The copolymer hybrid may be, for example, a fluorinated zwitterionic-modified polysiloxane (e.g., PDMS), polyolefin (e.g., PE, PP, PVC, or PVDF), polyester, polyurethane, polyurea, polycarbonate, polypeptide, polyimide, polyphosphazene, polyepoxy, phenolic polymer, polysulfone, or a polysulfide. In another instance, the bulk material is a hydrogel composition containing a network entanglement of one, two or three zwitterionic polymers selected from any of the zwitterionic polymer or hybrid copolymer compositions described above, wherein the zwitterionic polymer may be any of those described herein, such as those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II). In some embodiments, the hydrogel composition is a zwitterionic single-network (ZSN) hydrogel composition. In some embodiments, the hydrogel composition is a zwitterionic double-network (ZDN) hydrogel composition. In other embodiments, the hydrogel composition is a zwitterionic triple-network (ZTN) hydrogel composition. In particular embodiments, one, two, or all three of the zwitterionic polymers in the network are derived from olefinic monomers.

In some embodiments, the present disclosure is directed to zwitterionic double-network (ZDN) and triple-network (ZTN) hydrogel compositions containing a network entanglement of first, second, and third zwitterionic polymers. As well known in the art, a network entanglement results from the interpenetration of polymer chains and/or pendant groups and resulting loss of degree of freedom of the polymers without any formal bonding between the polymer chains and/or pendant groups engaged in the network entanglement. A zwitterionic double-network (ZDN) hydrogel composition contains two zwitterionic polymers engaged in a network entanglement. A zwitterionic triple-network (ZTN) hydrogel composition contains three zwitterionic polymers engaged in a network entanglement. Typically, at least one zwitterionic polymer out of the first, second, and third zwitterionic polymers is different than the other two (i.e., typically, the first, second, and third zwitterionic polymers are not all the same). In some embodiments, two of the zwitterionic polymers selected from the first, second, and third zwitterionic polymers are of the same type (i.e., within the same sub-formula) or have the same structure. In other embodiments, the first, second, and third zwitterionic polymers are not the same type or have different structures. As also well known, the term “hydrogel” refers to polymers that are swellable by absorption of a liquid, typically water, while maintaining their structure and not dissolving in the liquid. The zwitterionic polymer typically contains at least or greater than 10, 50, 100, 200, 300, 400, 500, 1000, 5000, 10,000, 50,000, or 100,000 units, or a number of units within a range bounded by any two of the foregoing values.

In some embodiments of the ZTN hydrogel, at least the second and third zwitterionic polymers contain at least 50 mol % zwitterionic moieties. In different embodiments, the second and/or third zwitterionic polymers independently contain precisely or at least 50, 55, 60, 65, 70, 75, 80, 85, 90, 95, or 100 mol % zwitterionic moieties, or a mol % within a range bounded by any two of the foregoing values (e.g., 50-100 mol %, 60-100 mol %, 70-100 mol %, 80-100 mol %, or 90-100 mol %). In different embodiments, at least the second and/or third zwitterionic polymers, and optionally the first zwitterionic polymer, independently contain precisely or at least 50, 55, 60, 65, 70, 75, 80, 85, 90, 95, or 100 mol % zwitterionic moieties, or a mol % within a range bounded by any two of the foregoing values (e.g., 50-100 mol %, 60-100 mol %, 70-100 mol %, 80-100 mol %, or 90-100 mol %).

The first zwitterionic polymer may contain precisely or at least, for example, 1, 2, 3, 4, 5, 10, 20, 30, 40, 50, 55, 60, 65, 70, 75, 80, 85, 90, 95, or 100 mol % zwitterionic moieties, or a mol % within a range bounded by any two of the foregoing values (e.g., 1-5 mol %, 1-10 mol %, 2-10 mol %, 3-10 mol %, 1-20 mol %, 1-30 mol %, 1-40 mol %, 1-50 mol %, 50-100 mol %, 60-100 mol %, 70-100 mol %, 80-100 mol %, or 90-100 mol %).

In some embodiments of the ZTN hydrogel, the first, second, and third zwitterionic polymers contain at least 50 mol % zwitterionic moieties. In different embodiments, one, two, or all (or each) of the first, second, and third zwitterionic polymers independently contain precisely or at least 50, 55, 60, 65, 70, 75, 80, 85, 90, 95, or 100 mol % zwitterionic moieties, or a mol % within a range bounded by any two of the foregoing values (e.g., 50-100 mol %, 60-100 mol %, 70-100 mol %, 80-100 mol %, or 90-100 mol %).

In some embodiments, the zwitterionic polymer is a betaine polymer. In other embodiments, the zwitterionic polymer is a poly(phosphatidylcholine) polymer, poly(trimethylamine N-oxide) polymer, poly(zwitterionic phosphatidylserine) polymer, or glutamic acid-lysine (EK)-containing polypeptide. In some embodiments, zwitterionic phosphatidyl serine comprises one neighboring positive charged moiety to balance the negative charge of the phosphoserine. In some embodiments, zwitterionic phosphatidyl serine comprises a compound as described in “De novo design of functional zwitterionic biomimetic material for immunomodulation” Science Advances, 29 May 2020, Vol. 6, Issue 22, (DOI: 10.1126/sciadv.aba0754) which is hereby incorporated by reference in its entirety. In some embodiments, a ZSN, ZDN or ZTN hydrogel includes a betaine polymer.

Some examples of betaine polymers include poly(carboxybetaine), poly(sulfobetaine), and poly(phosphobetaine) polymers. Suitable poly(carboxybetaine)s can be prepared from one or more monomers selected from, for example, carboxybetaine acrylates, carboxybetaine acrylamides, carboxybetaine vinyl compounds, carboxybetaine epoxides, and mixtures thereof. In one embodiment, the monomer is carboxybetaine methacrylate. Representative monomers for making carboxybetaine polymers useful in the invention include carboxybetaine methacrylates, such as 2-carboxy-N,N-dimethyl-N-(2′-methacryloyloxyethyl) ethanaminium inner salt; carboxybetaine acrylates; carboxybetaine acrylamides; carboxybetaine vinyl compounds; carboxybetaine epoxides; and other carboxybetaine compounds with hydroxyl, isocyanates, amino, or carboxylic acid groups. In a particular embodiment, the polymer is a poly(carboxybetaine methacrylate) (poly(CBMA)). In some embodiments, a ZSN, ZDN or ZTN hydrogel includes a poly(carboxybetaine), poly(sulfobetaine), or poly(phosphobetaine) polymer. In some embodiments of the ZTN hydrogel, the second and third zwitterionic polymers both have a poly(sulfobetaine) composition.

The present disclosure is also directed to a method for rendering a surface fouling-resistant and/or capable of fouling release. In the method, a surface is coated with any of the zwitterionic polymer compositions or copolymer hybrid compositions described above, wherein the zwitterionic polymer may be any of those described herein, such as those of the Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II), or a the surface may be coated with a ZSN, ZDN or ZTN hydrogel containing any one, two or three of these polymer or copolymer hybrid compositions. The surface may be part of an object designed to operate in a marine environment (e.g., ship hull) or medical environment (e.g., catheter, surgical device, or implant).

The present disclosure is also directed to a fouling-resistant object comprising: (i) an object having a surface; and (ii) a coating comprising any of the zwitterionic polymer or copolymer hybrid compositions described above, wherein the zwitterionic polymer may be any of those described herein, such as those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II) or a ZSN, ZDN or ZTN hydrogel of any one, two or three of these polymer or copolymer hybrid compositions on the surface. The coating may alternatively be a chemically crosslinked version of a single zwitterionic polymer described herein, or a chemically crosslinked version of a mixture of two or three zwitterionic polymers described herein, wherein the zwitterionic polymer may or may not be a copolymer hybrid as described above. The object may be designed to operate in a marine environment or medical environment. In some embodiments, the surface of the object possesses both a fouling-resistant and a fouling-release ability. The polymer compositions may be coated onto a surface by means well known in the art, including, for example, depositing as a solution or suspension, or producing the polymer on the surface in situ from appropriate monomer(s) deposited on the surface.

The present disclosure is also directed to zwitterionic nanoparticle compositions containing any of the zwitterionic polymer or copolymer hybrid compositions described above, wherein the zwitterionic polymer may be any of those described herein, such as those of Formulas (I), (Ia), (Ib), (Ic), (Ic-1), (Id), (Ie), (Ie-1), (Ie-1-1), (Ie-1-2), (Ie-1-3), (Ie-1-4), (If), (If-1), and (II) or a ZSN, ZDN or ZTN hydrogel of any one, two or three of these polymer or copolymer hybrid compositions within and/or on surfaces of nanoparticles. The nanoparticles may be composed entirely of the zwitterionic polymer(s) or copolymer hybrid, or the nanoparticles may have a core-shell structure in which the core has a solid structure (e.g., non-zwitterionic polymer or a metal oxide or metal) and the shell contains the zwitterionic polymer(s) or copolymer hybrid(s). The nanoparticles may have a size of, for example, 1, 5, 10, 20, 50, 100, 200, or 500 nm, or a size within a range between any two of these values. The polymeric nanoparticles may be produced by means well known in the art, such as by the double emulsion method.

In some embodiments, one or more zwitterionic polymers or copolymer hybrids are crosslinked. The crosslinking can be between the same polymer type or between different polymer types. In some embodiments, one or more crosslinked polymers are in a ZSN, ZDN or ZTN hydrogel. In some network hydrogels, only or at least the first zwitterionic polymer is crosslinked, which may be between chains of the first zwitterionic polymer, or between chains of the first and second zwitterionic polymers, or both. In other network hydrogels, only or at least the second zwitterionic polymer is crosslinked, which may be between chains of the second zwitterionic polymer, or between chains of the second and third zwitterionic polymers, or both. In yet other network hydrogels, only or at least the third zwitterionic polymer is crosslinked, which may be between chains of the third zwitterionic polymer, or between chains of the second and third zwitterionic polymers, or both. In some embodiments, all of the zwitterionic polymers in a network hydrogel are crosslinked.

Crosslinking of polymer chains is typically achieved by including a crosslinking monomer, such as a bis(acrylamide), bis(acrylate), or bis(methacrylate), in the polymerization reaction. In some embodiments, the crosslinking monomer is not charged or zwitterionic. In other embodiments, the crosslinking monomer is charged or zwitterionic, such as in carboxybetaine dimethacrylate. Some specific examples of crosslinking monomers include the following:

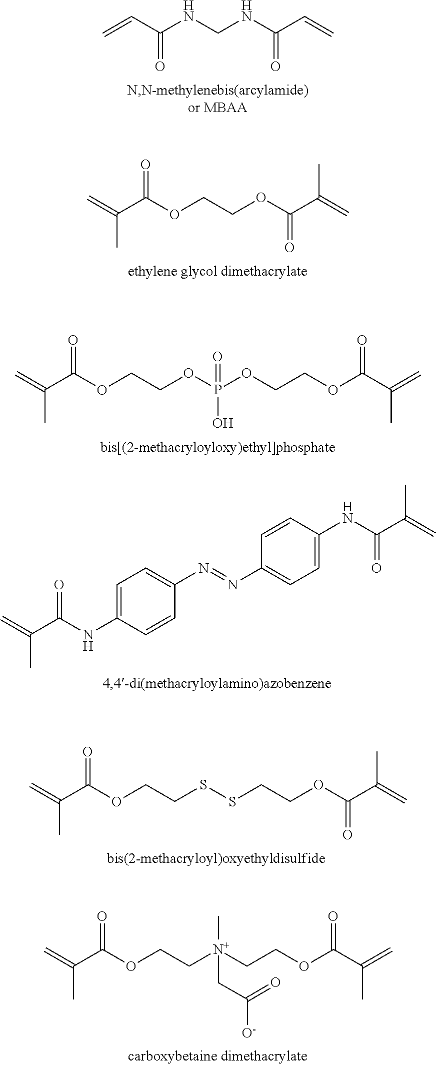

In a first set of particular embodiments, the ZTN hydrogel contains a first zwitterionic polymer having the structure of Formula (Ic) or (Ic-1), or more particularly, pTMAO, which may be crosslinked or uncrosslinked. In other particular embodiments, the ZTN hydrogel contains a first zwitterionic polymer having any of the structures of Formulas (Id), (Ie), (Ie-1), (Ie-1-1), (Ie-1-2), (Ie-1-3), (Ie-1-4), (If), (If-1), and (II), or more particularly, pSB or pCB, any of which may be crosslinked or uncrosslinked. Alternatively, a ZSN or ZDN hydrogel may contain a zwitterionic polymer having the structure of Formula (Ic) or (Ic-1), or more particularly, pTMAO, which may be crosslinked or uncrosslinked.

In a second set of particular embodiments, the ZTN hydrogel contains a second zwitterionic polymer having the structure of Formula (Ic) or (Ic-1), or more particularly, pTMAO, which may be crosslinked or uncrosslinked. In other particular embodiments, the ZTN hydrogel contains a second zwitterionic polymer having any of the structures of Formulas (Id), (Ie), (Ie-1), (Ie-1-1), (Ie-1-2), (Ie-1-3), (Ie-1-4), (If), (If-1), and (II), or more particularly, pSB or pCB, any of which may be crosslinked or uncrosslinked.

In a third set of particular embodiments, the ZTN hydrogel contains a third zwitterionic polymer having the structure of Formula (Ic) or (Ic-1), or more particularly, pTMAO, which may be crosslinked or uncrosslinked. In other particular embodiments, the ZTN hydrogel contains a third zwitterionic polymer having any of the structures of Formulas (Id), (Ie), (Ie-1), (Ie-1-1), (Ie-1-2), (Ie-1-3), (Ie-1-4), (If), (If-1), and (II), or more particularly, pSB or pCB, any of which may be crosslinked or uncrosslinked.

In some embodiments, any one of the first, second, or third particular embodiments provided above are combined. For a ZDN hydrogel, one or both of the entangled polymers are fluorinated. For a ZTN hydrogel, one, two, or all three of the entangled polymers are fluorinated. In a first particular example, the first zwitterionic polymer may be pTMAO and the second zwitterionic polymer may be pSB, pCB, or pTMAO. In a second particular example, the first zwitterionic polymer may be pSB and the second zwitterionic polymer may be pSB, pCB, or pTMAO. In a third particular example, the first zwitterionic polymer may be pCB and the second zwitterionic polymer may be pSB, pCB, or pTMAO. In any of the foregoing first, second, and third particular examples, the third zwitterionic polymer may be selected from, for example, pSB, pCB, or pTMAO. The resulting ZTN hydrogel may be, for example, pTMAO/pSB/pSB, pTMAO/pSB/pCB, pTMAO/pCB/pCB, pTMAO/pCB/pSB, pTMAO/pSB/pTMAO, pTMAO/pCB/pTMAO, pTMAO/pTMAO/pSB, pTMAO/pTMAO/pCB, pCB/pSB/pSB, pCB/pSB/pCB, pCB/pCB/pSB, pCB/pSB/pTMAO, pCB/pCB/TMAO, pCB/pSB/pTMAO, pCB/pTMAO/pCB, pCB/pTMAO/pSB, pSB/pCB/pCB, pSB/pCB/pSB, pSB/pSB/pCB, pSB/pCB/pTMAO, pSB/pSB/TMAO, pSB/pCB/pTMAO, pSB/pTMAO/pSB, or pSB/pTMAO/pCB, wherein each of the foregoing examples has the nomenclature (FZP/SZP/TZP), wherein FZP=first zwitterionic polymer, SZP=second zwitterionic polymer, and TZP=third zwitterionic polymer.

The zwitterionic polymers can be prepared by any suitable polymerization method, such as vinyl-addition (free radical) polymerization, atom transfer radical polymerization (ATRP), and reversible addition fragmentation chain transfer (RAFT) polymerization. Any of the known radical initiators, including photoinitiators, for polymerizing such monomers, may be used. The initiator (or photoinitiator) may be, for example, azobisisobutyronitrile (AIBN), 2-hydroxy-2-methylpropiophenone, or 2,2-dimethoxy-2-phenylacetophenone. For example, sulfobetaine methacrylate monomer may be polymerized, typically by a vinyl-addition process, to form poly(sulfobetaine methacrylate). Alternatively, a non-zwitterionic precursor polymer may first be produced, followed by conversion of the non-zwitterionic precursor polymer to a zwitterionic polymer. For example, an amino-containing polymer, such as poly(2-aminoethylmethacrylate), may be reacted with a haloalkyl molecule containing an anionic group (e.g., carboxylate or sulfonate) to result in conversion of the amino groups to ammonium groups attached indirectly to the anionic group.

In another aspect, the present disclosure is directed to a method for producing a ZSN, ZDN or ZTN hydrogel compositions described above. In one method, a first zwitterionic polymer is obtained (provided), either commercially or by synthesis. If by synthesis, the first zwitterionic polymer (or hybrid copolymer) can be produced by polymerization of suitable monomers, in the absence or presence of a crosslinking monomer, typically in a mold or on the surface of a substrate. Alternatively, the first zwitterionic polymer may be affixed to (or coated onto) the surface of a substrate. In some embodiments, the first zwitterionic polymer (or hybrid copolymer) is coated onto the surface of an object for which fouling is to be inhibited or prevented. The object may be, for example, a metallic fin, propeller, or structural material designed for use in an underwater (typically, seawater) or other saline environment. The saline environment is typically ocean water, but may be another type of brackish water, either from a natural or industrial source. In some embodiments, a ZSN hydrogel is produced by crosslinking of a single zwitterionic polymer or copolymer hybrid thereof.

In a second step (step ii), a first precursor solution is absorbed into the first zwitterionic polymer, wherein the first precursor solution includes or exclusively contains a first zwitterionic monomer species (and optionally, a non-zwitterionic monomer, as described earlier above) dissolved in a solvent. The solvent may be, for example, water or an aqueous based solvent mixture (e.g., alcohol-water mixture).

In a third step (step iii), the first zwitterionic monomer species is polymerized to form a second zwitterionic polymer while absorbed in the first zwitterionic polymer. When the second zwitterionic polymer is different from the first zwitterionic polymer, the end result is formation of a zwitterionic double-network (ZDN) hydrogel composition containing the second zwitterionic polymer entangled in the first zwitterionic polymer. Alternatively, the first zwitterionic monomer species is of the same type used to produce the first zwitterionic polymer, in which case, after polymerization of the first zwitterionic monomer, the second zwitterionic polymer is the same as the first zwitterionic polymer and a ZSN entangled hydrogel results. In some embodiments, only a ZSN or ZDN hydrogel is desired, in which case the following steps for producing a ZTN hydrogel are not performed. In some embodiments, after this two-step synthesis, the ZSN or ZDN hydrogels are soaked in water for precisely or at least 1, 2, 3, 4, or 5 days to reach equilibrium, before optionally proceeding with the fourth step (below).

In an optional fourth step (step iv), a second precursor solution is absorbed into the ZDN hydrogel composition, wherein the second precursor solution includes or exclusively contains a second zwitterionic monomer species dissolved in a solvent. The solvent may be, for example, water or an aqueous based solvent mixture (e.g., alcohol-water mixture), and may be the same or different as the solvent used in step (ii).

In an optional fifth step (step v), the second zwitterionic monomer species is polymerized to form a third zwitterionic polymer while absorbed in the ZDN hydrogel composition, which results in formation of the zwitterionic triple-network (ZTN) hydrogel composition containing the third zwitterionic polymer entangled in the ZDN hydrogel composition. The third zwitterionic polymer is typically entangled with both the first and second zwitterionic polymers in the ZTN hydrogel composition.

The first, second, and third zwitterionic polymers used in the method typically contain at least 50 mol % zwitterionic moieties. In different embodiments, one, two, or all (or each) of the first, second, and third zwitterionic polymers independently contain precisely or at least 50, 55, 60, 65, 70, 75, 80, 85, 90, 95, or 100 mol % zwitterionic moieties, or a mol % within a range bounded by any two of the foregoing values (e.g., 50-100 mol %, 60-100 mol %, 70-100 mol %, 80-100 mol %, or 90-100 mol %).

Typically, in the method, at least one zwitterionic polymer out of the first, second, and third zwitterionic polymers is different than the other two (i.e., typically, the first, second, and third zwitterionic polymers are not all the same). In some embodiments, two of the zwitterionic polymers selected from the first, second, and third zwitterionic polymers have the same structure. In other embodiments, the first, second, and third zwitterionic polymers have different structures.

Notably, the entire process for producing the ZSN, ZDN or ZTN hydrogel composition, as described above, may be conducted on the surface of an object for which fouling is to be inhibited or prevented, starting with producing a coating of the first zwitterionic polymer on the surface. In some embodiments, before placing the coating of the first zwitterionic polymer on the surface, the surface is cleaned or pretreated to result in a stronger bond between the polymer and the surface.

As indicated earlier above, aside from possessing excellent resistance to fouling and mechanical properties, the ZSN, ZDN or ZTN hydrogel compositions described herein also surprisingly exhibit exceptional (high) fouling-release ability. The term “high fouling-release” is used herein to mean that bio-foulants can be removed from the ZDN or ZTN hydrogel compositions at low waterjet pressure, e.g., no more than or below 10 or 20 Psia. The foregoing property is a significant advance since zwitterionic polymer networks of the art are typically not capable of such low-pressure release of bio-foulants.

In another aspect, the present disclosure is directed to a method of producing a carboxybetaine (CB) monomer of the formula

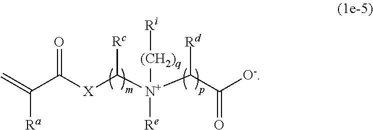

The method of producing a CB monomer comprises a first step as follows:
(i) producing an intermediate (Int-1) by the following reaction scheme:

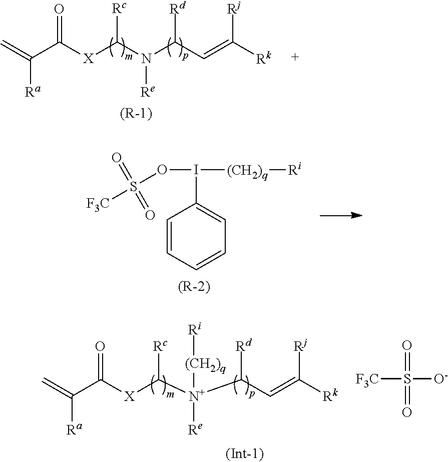

wherein:
R^{a} is H or an alkyl group containing 1-3 carbon atoms, as described above;
X is O or NRW, wherein R^{b} is H or an alkyl group containing 1-3 carbon atoms, as described above;
m is an integer of precisely or at least 1, as described above;
p is an integer of precisely or at least 1, as described above;
R^{c} and R^{d} are independently selected from hydrogen atom, hydrocarbon group, and fluorinated hydrocarbon group containing 1-12 carbon atoms and at least one fluorine atom;
R^{e} is selected from R^{a}; q is an integer of precisely or at least 1; Rⁱ is a fluorinated hydrocarbon group containing 1-12 carbon atoms and at least one fluorine atom; and
R^{j} and R^{k} are independently selected from hydrogen atom, alkyl group containing 1-3 carbon atoms, silyl groups, and stannyl groups;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

The above reaction in step (i) may be conducted in a polar aprotic solvent, such as methylene chloride, THF, or diethyl ether, typically at reduced temperature, e.g., 0° C. or lower.

The method of producing a CB monomer comprises a second step as follows: (ii) oxidatively cleaving the intermediate (Int-1) to produce the carboxybetaine monomer of the formula (1e-5). The reaction in step (ii) may be conducted in a polar aprotic solvent, such as DMF, methylene chloride, THF, or diethyl ether, at room temperature or a reduced temperature, e.g., 0° C. or lower. The oxidative cleavage may be effected by, for example, osmium tetroxide, oxone, ozone, or a combination of any two of these.

The method of producing a CB monomer, described above, may also include separate synthetic steps for producing any of the reactants R-1 and R-2. The following is an exemplary process for producing the CB monomer, including exemplary means for producing R-1 and R-2:

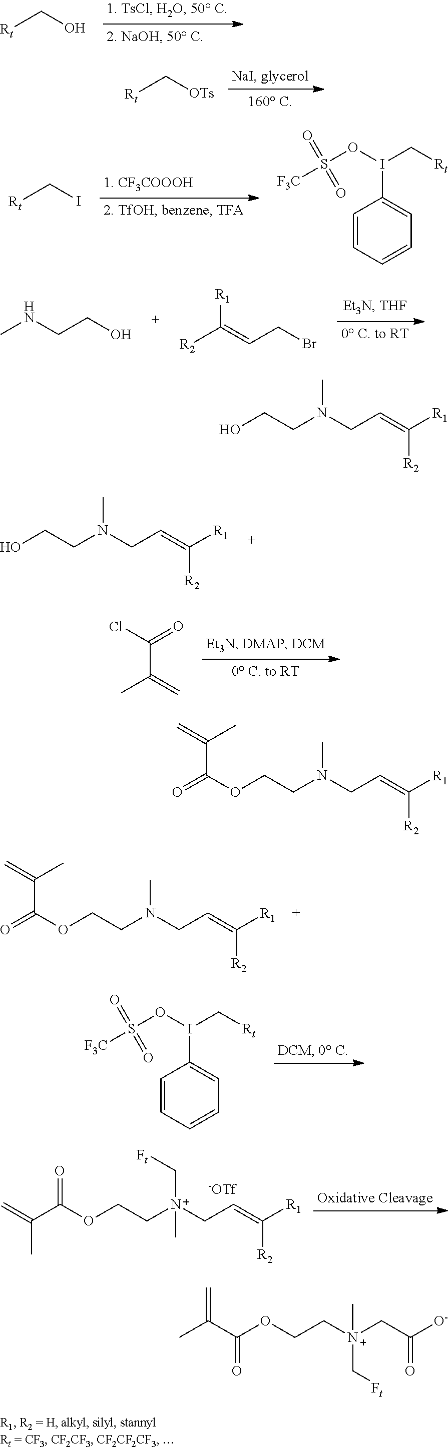

In another aspect, the present disclosure is directed to a method of producing a trimethylammonium oxide (TMAO) monomer of the formula

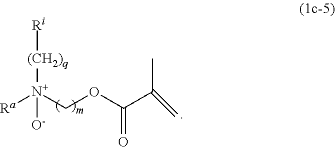

The method of producing a TMAO monomer comprises a first step as follows:
(i) producing an intermediate (Int-2) by the following reaction scheme:

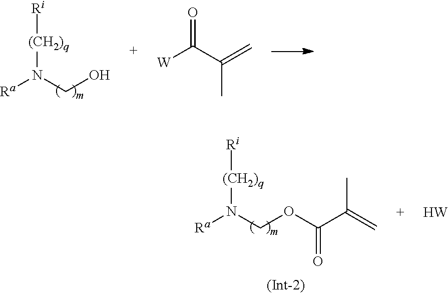

wherein: R^{a} is H or an alkyl group containing 1-3 carbon atoms; m is an integer of precisely or at least 1; q is an integer of precisely or at least 1; Rⁱ is a fluorinated hydrocarbon group containing 1-12 carbon atoms and at least one fluorine atom; and W is Cl or Br.

The above reaction in step (i) may be conducted in a polar aprotic solvent, such as methylene chloride, THF, or diethyl ether, either at room temperature or a reduced temperature, e.g., 0° C. or lower, and typically in the presence of an amine, such as triethylamine, 4-dimethylaminopyridine (DMAP), or both.

The method of producing a TMAO monomer comprises a second step as follows: (ii) oxidizing the intermediate (Int-2) to produce the TMAO monomer of the formula (1c-5). The reaction in step (ii) may be conducted in a polar aprotic solvent, such as DMF, methylene chloride, THF, or diethyl ether, at a reduced temperature, e.g., 0° C. or lower. The oxidation may be effected by, for example, a perbenzoic acid, such as m-chloroperoxybenzoic acid (mCPBA).

The method of producing a TMAO monomer, described above, may also include separate synthetic steps for producing the reactant R-3. The following is an exemplary process for producing the TMAO monomer, including exemplary means for producing R-3:

The present disclosure is also directed to the Int-1 and Int-2 compositions.

Examples have been set forth below for the purpose of illustration and to describe the best mode of the invention at the present time. However, the scope of this invention is not to be in any way limited by the examples set forth herein.

### EXAMPLES

### Synthesis of Compounds

The following monomeric compounds were synthesized:

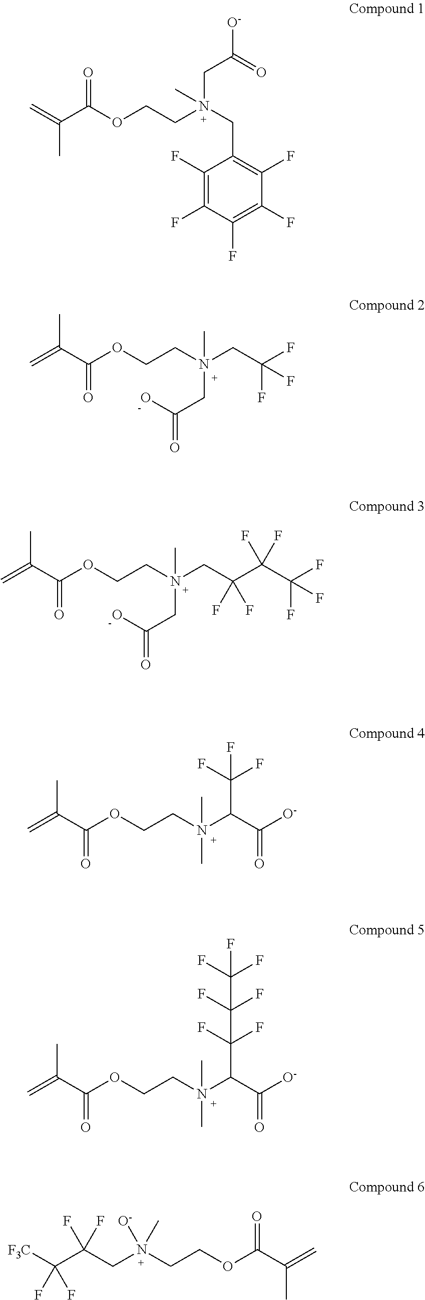

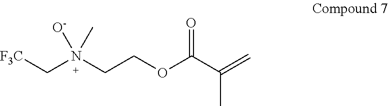

### Synthesis of Compound 1

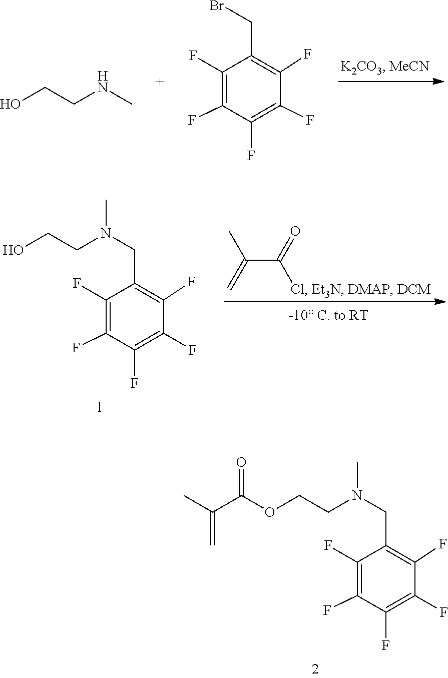

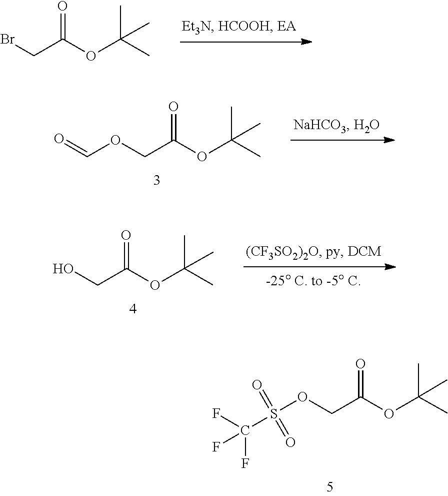

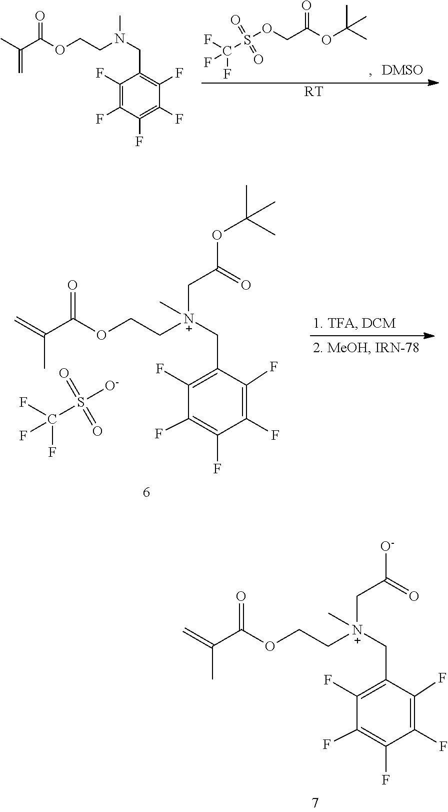

2-(methyl((perfluorophenyl)methyl)amino)ethan-1-ol (1). A flame dried 500 mL Schlenk flask was charged with anhydrous potassium carbonate (52.96 g), 200 mL of dry acetonitrile, and 2-(methylamino)ethan-1-ol (13.70 g). The mixture was cooled to −5° C. in an ice-salt bath, and 1-(bromomethyl)-2,3,4,5,6-pentafluorobenzene (50.00 g) was added dropwise. The reaction was allowed to come to room temperature over a period of one hour and stirred under N₂ for 8 hours. The solvent was removed in vacuo, dissolved in diethyl ether, filtered, and purified using a plug of silica. Yield was 38.83 g (83%) of a colorless oil.

2-(methyl((perfluorophenyl)methyl)amino)ethyl methacrylate (2). To a flame dried 2 L flask was added 1 L of dry dichloromethane, 4-dimethylaminopyridine (1.74 g), 2-(methyl((perfluorophenyl)methyl)amino)ethan-1-ol (36.31 g), and freshly distilled triethylamine (79.33 mL) dried over potassium hydroxide. The mixture was cooled to −5° C. in an ice-salt bath and freshly distilled methacryloyl chloride (27.80 mL) was added over 2 hours via syringe pump. The reaction was allowed to stir under N₂ for 6 hours after coming to room temperature. The solvent was removed in vacuo, and the product was purified using a plug of basic activated alumina. Yield was 42.61 g (93%) of a colorless oil.

tert-butyl 2-(formyloxy)acetate (3). To a 2 L flask was added 1 L ethyl acetate, freshly distilled triethylamine (145 mL), and formic acid (37.5 mL). The mixture was stirred for 10 minutes and tert-butyl 2-bromoacetate (95 mL) was added slowly. After 24 hours at room temperature, the precipitates were filtered and washed with diethyl ether. The organics were washed with brine, dried with magnesium sulfate, and removed in vacuo. Yield was 69.29 g (76%) of a colorless liquid.

tert-butyl 2-hydroxyacetate (4). To a 2 L flask was added tert-butyl 2-(formyloxy)acetate (62.00 g) and sodium bicarbonate (65.04 g) in 1 L of water. The suspension was stirred for 48 hours and extracted with chloroform. The organics were washed with brine and dried with magnesium sulfate. The product was fractionally distilled under vacuum to yield 47.83 g (94%) of a colorless liquid.

tert-butyl 2-(((trifluoromethyl)sulfonyl)oxy)acetate (5). A flame dried 1 L Schlenk flask was charged with 500 mL of dry dichloromethane and anhydrous pyridine (23.76 mL) purified by distillation and dried over molecular sieves. The mixture was cooled to −25° C. using a recirculating chiller, and trifluoromethanesulfonic anhydride (43.83 mL) was added via syringe pump over 3 hours. Tert-butyl 2-hydroxyacetate (30.00 g) was then added slowly, and the reaction was allowed to come to −5° C. and stirred for 3 hours under N₂. 500 mL of ice-cold water was added, and the product was quickly extracted using dichloromethane. The extracts were washed with brine and dried with sodium sulfate. The product was purified via filtration through silica gel to give 27.24 g (45%) of a colorless oil.

2-(tert-butoxy)-N-(2-(methacryloyloxy)ethyl)-N-methyl-2-oxo-N-((perfluorophenyl)methyl)ethan-1-aminium trifluoromethanesulfonate (6). A flame dried 200 mL Schlenk flask was charged with 2-(methyl((perfluorophenyl)methyl)amino)-ethyl methacrylate (29.34 g), 90 mL of dry dimethyl sulfoxide, and butylated hydroxytoluene (5 mg). The mixture was cooled to 0° C. in an ice bath and tert-butyl 2-(((trifluoromethyl)sulfonyl)oxy)acetate (24.00 g) was added dropwise. The reaction was brought to room temperature and stirred for 24 hours under N₂. It was then poured onto water, impurities were removed via extraction with tert-butyl methyl ether, and the product was extracted with n-butanol. The solvent was removed in vacuo to yield 50.14 g (94%) of a white foam.

2-((2-(methacryloyloxy)ethyl)(methyl)((perfluorophenyl)methyl)ammonio)-acetate (7). To a flame dried 1 L flask was added 500 mL dry dichloromethane and 2-(tert-butoxy)-N-(2-(methacryloyloxy)ethyl)-N-methyl-2-oxo-N-((perfluorophenyl)methyl)ethan-1-aminium trifluoromethanesulfonate (51.35 g). The mixture was cooled to 0° C. in an ice bath and trifluoroacetic acid (38 mL) was added slowly. The reaction was stirred under N₂ for 4 hours at room temperature. Volatiles were removed in vacuo, and the product was dissolved in a minimum amount of methanol. A column of RN-78 resin was washed with methanol before the product was added and eluted using methanol. Yield was 36.26 g (97%) of a white foam.

### Alternative Synthesis of Compound 1

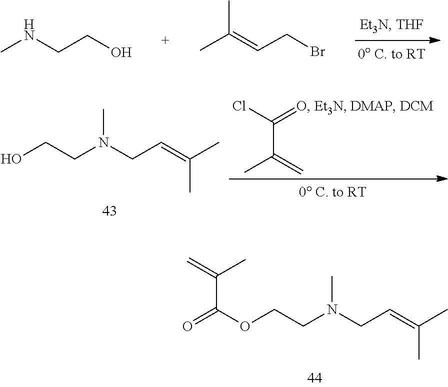

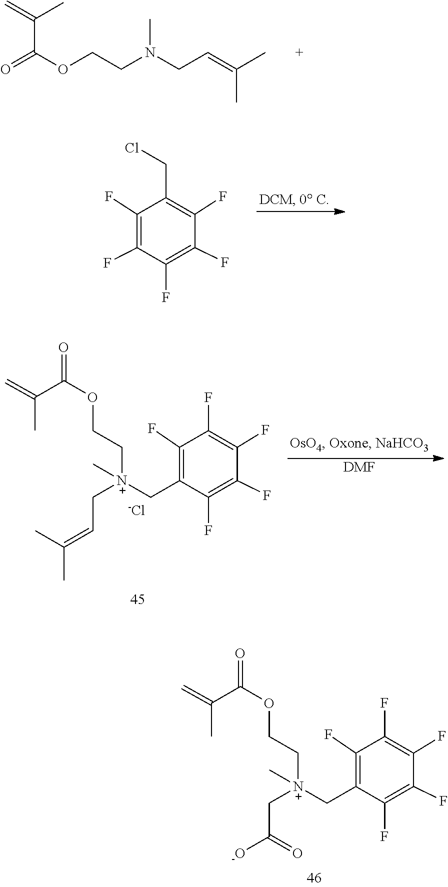

2-((2-(methacryloyloxy)ethyl)(methyl)((perfluorophenyl)methyl)ammonio)-acetate (46). 500 mL of dimethylformamide in a 1 L flask was mixed with N-(2-(methacryloyloxy)ethyl)-N,3-dimethyl-N-((perfluorophenyl)methyl)but-2-en-1-aminium chloride (50.00 g). Osmium tetroxide (60.0 mg) was added, and the mixture was allowed to stir for 15 minutes. A blend of Oxone (143.69 g) and sodium bicarbonate (39.27 g) was added portion-wise, and the reaction was allowed to stir for 7 hours. The mixture was filtered, and the solvent removed in vacuo. The residue was taken up in trifluoroethanol, filtered, and dried in vacuo to yield 29.73 g (90%) of a white foam. Spectral data matched the prior synthesis of compound 1.

### Synthesis of Compound 2

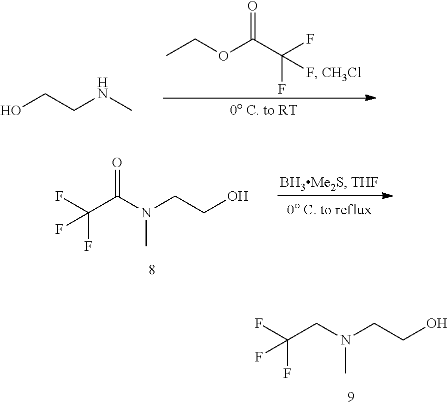

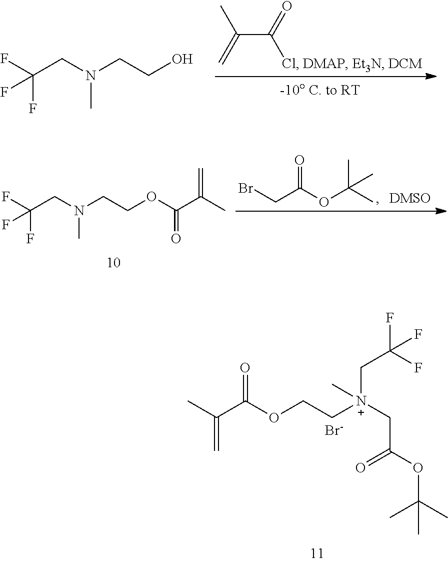

2,2,2-trifluoro-N-(2-hydroxyethyl)-N-methylacetamide (8). A flame dried 500 mL Schlenk flask was charged with 350 mL of dry dichloromethane and 2-(methylamino)ethan-1-ol (25.17 g). The mixture was cooled to −5° C. in an ice-salt bath, and ethyl 2,2,2-trifluoroacetate (50.00 g) was added via syringe pump over 2 hours. The reaction was allowed to come to room temperature and stirred under N₂ for 12 hours. The solvent was washed with 0.1 N HCl, brine, and dried with magnesium sulfate. Yield was 51.62 g (90%) of a colorless oil.

2-(methyl(2,2,2-trifluoroethyl)amino)ethan-1-ol (9). To a flame dried 250 mL Schlenk flask was added 100 mL dry tetrahydrofuran and 2,2,2-trifluoro-N-(2-hydroxyethyl)-N-methylacetamide (25.00 g). The mixture was cooled to −5° C. in an ice-salt bath. Borane dimethylsulfide (20.8 mL) was added via syringe pump over 2 hours. After stirring for 8 hours at −5° C. under N₂, methanol was added dropwise via dropping funnel to quench the reaction. The mixture was refluxed for 3 hours to break the borane-amine complex. The solvent was removed in vacuo. Fractional distillation in vacuo provided 19.98 g (87%) of a colorless oil.

2-(methyl(2,2,2-trifluoroethyl)amino)ethyl methacrylate (10). To a flame dried 2 L flask was added 1 L of dry dichloromethane, 4-dimethylaminopyridine (1.55 g), 2-(methyl(2,2,2-trifluoroethyl)amino)ethan-1-ol (20.00 g), and freshly distilled triethylamine (71.10 mL) dried over potassium hydroxide. The mixture was cooled to −5° C. in an ice-salt bath and freshly distilled methacryloyl chloride (24.87 mL) was added over 2 hours via syringe pump. The reaction was allowed to stir under N₂ for 6 hours after coming to room temperature. The solvent was removed in vacuo, and the product was purified using a plug of basic activated alumina. Yield was 25.51 g (89%) of a colorless oil.

N-(2-(tert-butoxy)-2-oxoethyl)-2,2,2-trifluoro-N-(2-(methacryloyloxy)ethyl)-N-methylethan-1-aminium bromide (11). A flame dried 200 mL Schlenk flask was charged with 2-(methyl(2,2,2-trifluoroethyl)amino)ethyl methacrylate (25.00 g), 100 mL dry dimethyl sulfoxide, and butylated hydroxytoluene (5 mg). The mixture was cooled to 0° C. in an ice bath and tert-butyl bromoacetate (21.65 g) was added dropwise. The reaction was brought to room temperature and stirred for 120 hours under N₂. It was then poured onto water, impurities were removed via extraction with tert-butyl methyl ether, and the product was extracted with n-butanol. The solvent was removed in vacuo, and the residue was purified using a silica plug. The solvent was removed in vacuo to yield 4.20 g (9%) of a white foam.

### Alternative Synthesis of Compound 2

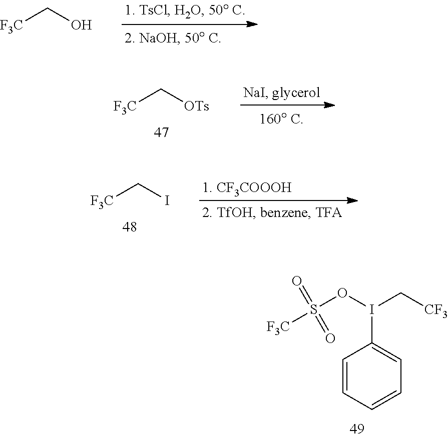 [SMILES: OCC(F)(F)F ; Cc1ccc(S(=O)(=O)OCC(F)(F)F)cc1 ; FC(F)(F)CI ; O=S(=O)(OI(CC(F)(F)F)c1ccccc1)C(F)(F)F]

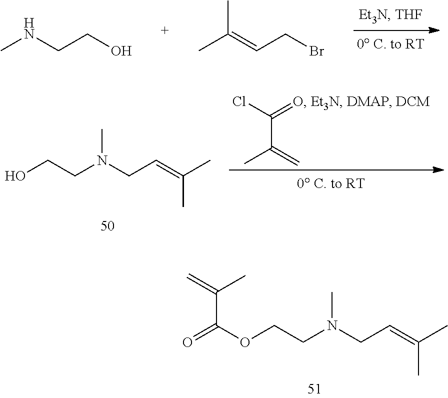

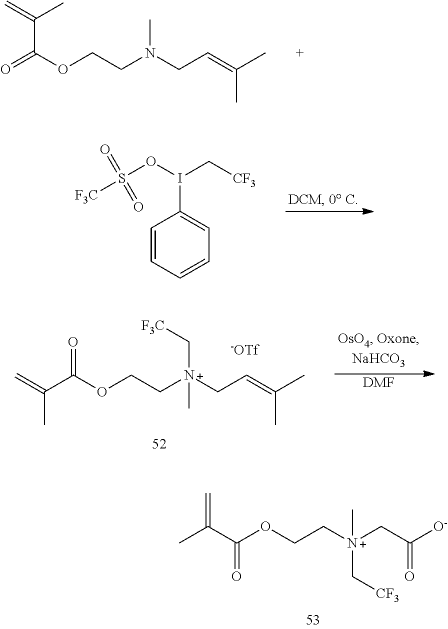

2-((2-(methacryloyloxy)ethyl)(methyl)(2,2,2-trifluoroethyl)ammonio)acetate (53). 500 mL of dimethylformamide in a 1 L flask was mixed with N-(2-(methacryloyloxy)ethyl)-N,3-dimethyl-N-(2,2,2-trifluoroethyl)but-2-en-1-aminium trifluoromethanesulfonate (51.82 g). Osmium tetroxide (60.0 mg) was added, and the mixture was allowed to stir for 15 minutes. A blend of Oxone (143.69 g) and sodium bicarbonate (39.27 g) was added portion-wise, and the reaction was allowed to stir for 14 hours. The mixture was filtered, and the solvent removed in vacuo. The residue was taken up in trifluoroethanol, filtered, and dried in vacuo to yield 28.50 g (86%) of a white foam. Spectral data matched the prior synthesis of compound 2.

### Synthesis of Compound 3

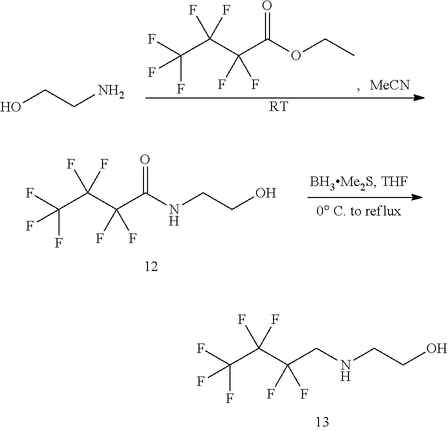

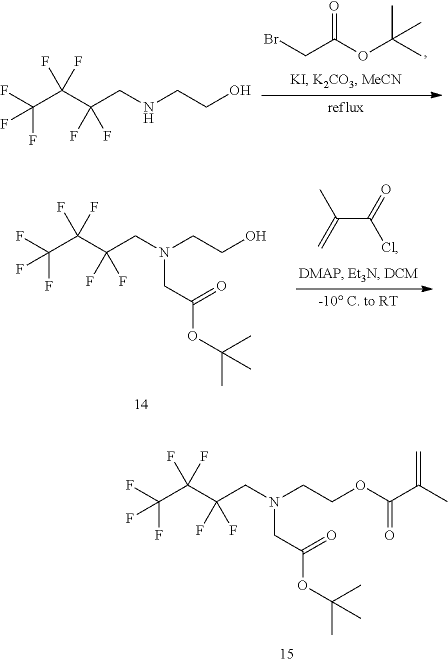

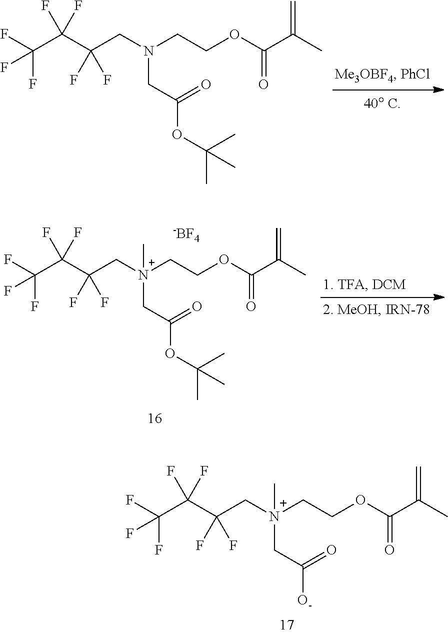

2,2,3,3,4,4,4-heptafluoro-N-(2-hydroxyethyl)butanamide (12). A 500 mL flask was charged with 100 mL of acetonitrile and 2-aminoethan-1-ol (15.77 g). The mixture was cooled to 0° C. in an ice bath, and ethyl 2,2,3,3,4,4,4-heptafluorobutanoate (75.00 g) was added slowly via dropping funnel. The reaction was allowed to come to room temperature and stirred for 16 hours. The solvent was removed to yield 63.59 g (96%) of a light yellow solid.

2-((2,2,3,3,4,4,4-heptafluorobutyl)amino)ethan-1-ol (13). To a flame dried 3-necked 1 L flask was added 500 mL of dry THF and 2,2,3,3,4,4,4-heptafluoro-N-(2-hydroxyethyl)butanamide (63.59 g). The mixture was cooled to −5° C. using an ice-salt bath. Borane dimethylsulfide (70.37 mL) was added via syringe pump over 4 hours. After stirring for 3 hours at −5° C. under N₂, the reaction was brought to reflux for an additional 4 hours. Once the reaction cooled to room temperature, methanol was added dropwise to quench the reaction. The mixture was refluxed for 5 hours to break the borane-amine complex. The solvent was removed in vacuo and the residue was taken up in dichloromethane, filtered though silica gel, and distilled fractionally in vacuo to give 54.37 g (90%) of a colorless oil.

tert-butyl N-(2,2,3,3,4,4,4-heptafluorobutyl)-N-(2-hydroxyethyl)glycinate (14). To a flame dried 1 L 3-necked flask was added anhydrous potassium carbonate (25.58 g), potassium iodide (30.73 g), 2-((2,2,3,3,4,4,4-heptafluorobutyl)amino)ethan-1-ol (45.00 g), and 500 mL of dry acetonitrile. A syringe pump was used to add tert-butyl bromoacetate (36.10 g) over 1 hour. The mixture was refluxed for 36 hours under N₂. The solvent was removed in vacuo, and the residue was dissolved in 10% ethyl acetate/hexane and filtered through silica gel to give 52.28 g (79%) of a colorless oil.

2-((2-(tert-butoxy)-2-oxoethyl)(2,2,3,3,4,4,4-heptafluorobutyl)amino)ethyl methacrylate (15). To a flame dried 2 L flask was added 1 L of dry dichloromethane, 4-dimethylaminopyridine (1.41 g), tert-butyl N-(2,2,3,3,4,4,4-heptafluorobutyl)-N-(2-hydroxyethyl)glycinate (41.30 g), and freshly distilled triethylamine (64.45 mL) dried over KOH. The mixture was cooled to −5° C. in an ice-salt bath and freshly distilled methacryloyl chloride (22.57 mL) was added over 3 hours via syringe pump. The reaction was allowed to stir under N₂ for 9 hours after coming to room temperature. The solvent was removed in vacuo, and the product was purified using a plug of basic activated alumina. Yield was 41.64 g (85%) of a colorless oil.

N-(2-(tert-butoxy)-2-oxoethyl)-2,2,3,3,4,4,4-heptafluoro-N-(2-(methacryloyloxy)ethyl)-N-methylbutan-1-aminium tetrafluoroborate (16). To a flame dried 200 mL Schlenk flask was added 100 mL of rigorously dried chlorobenzene prepared by drying with 4 Å molecular sieves three times in sequence and 2-((2-(tert-butoxy)-2-oxoethyl)(2,2,3,3,4,4,4-heptafluorobutyl)amino)ethyl methacrylate (40.00 g). Trimethyloxonium tetrafluoroborate was purified by washing with dichloromethane under N₂, followed by dry diethyl ether, and dried in vacuo. Trimethyloxonium tetrafluoroborate (27.82 g) was added quickly in portions to the reaction, and the mixture was heated to 40° C. for 24 hours. The reaction was quenched with cold saturated sodium bicarbonate and extracted with dichloromethane. The solvent was removed in vacuo, and the residue purified using a neutral activated alumina plug. Yield was 36.69 g (74%) of a white foam.

2-((2,2,3,3,4,4,4-heptafluorobutyl)(2-(methacryloyloxy)ethyl)(methyl)-ammonio)acetate (17). To a flame dried 500 mL flask was added 300 mL dry DCM and N-(2-(tert-butoxy)-2-oxoethyl)-2,2,3,3,4,4,4-heptafluoro-N-(2-(methacryloyloxy)ethyl)-N-methylbutan-1-aminium tetrafluoroborate (40.00 g). The mixture was cooled to 0° C. in an ice bath and trifluoroacetic acid (30 mL) was added slowly. The reaction was stirred under N₂ for 4 hours at room temperature. Volatiles were removed in vacuo, and the product was dissolved in a minimum amount of methanol. The product was purified using a column of IRN-78 resin that was washed with methanol. Yield was 28.50 g (98%) of a white foam. ¹H NMR (500 MHz, CDCl₃): δ 6.12 (s, 1H), 5.70 (s, 1H), 5.10-4.90 (m, 2H), 4.87-4.55 (m, 8H), 3.83 (s, 3H), 1.95 (s, 3H). ¹⁹F NMR (470 MHz, CDCl₃): δ −80.78 (m), −118.63 (m), −127.46 (m).

### Alternative Synthesis of Compound 3

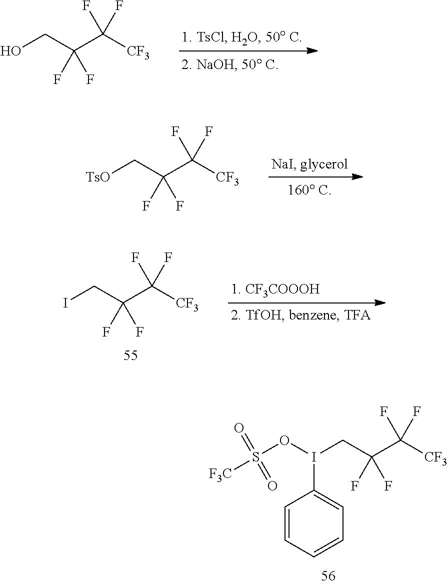 [SMILES: OCC(F)(F)C(F)(F)C(F)(F)F ; Cc1ccc(S(=O)(=O)OCC(F)(F)C(F)(F)C(F)(F)F)cc1 ; FC(F)(F)C(F)(F)C(F)(F)CI ; O=S(=O)(OI(CC(F)(F)C(F)(F)C(F)(F)F)c1ccccc1)C(F)(F)F]

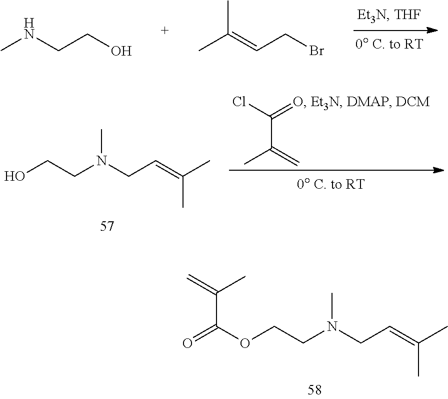

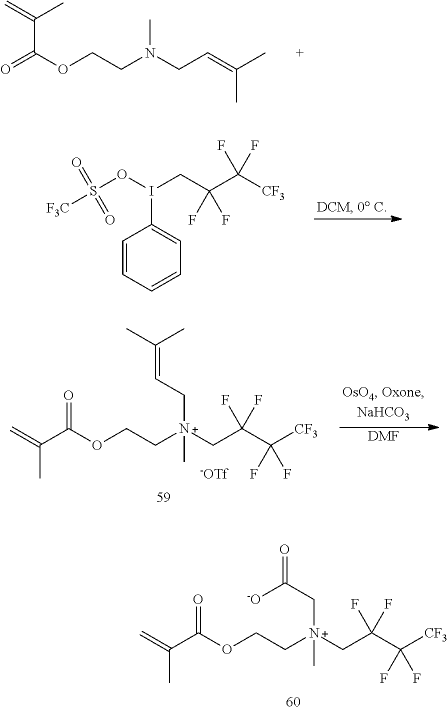

2-((2,2,3,3,4,4,4-heptafluorobutyl)(2-methacryloyloxy)ethyl)(methyl)-ammonio)acetate (60). 500 mL of dimethylformamide in a 1 L flask was mixed with N-(2,2,3,3,4,4,4-heptafluorobutyl)-N-(2-(methacryloyloxy)ethyl)-N,3-dimethylbut-2-en-1-aminium trifluoromethanesulfonate (63.17 g). Osmium tetroxide (60.0 mg) was added, and the mixture was allowed to stir for 15 minutes. A blend of Oxone (143.69 g) and sodium bicarbonate (39.27 g) was added portion-wise, and the reaction was allowed to stir for 17 hours. The mixture was filtered, and the solvent removed in vacuo. The residue was taken up in trifluoroethanol, filtered, and dried in vacuo to yield 37.94 g (85%) of a white foam. Spectral data matched the prior synthesis of compound 3.

### Synthesis of Compound 4

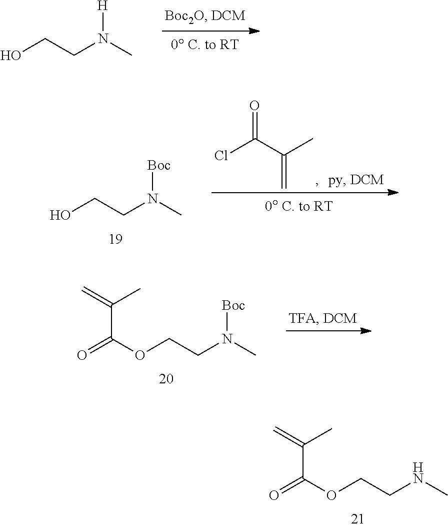

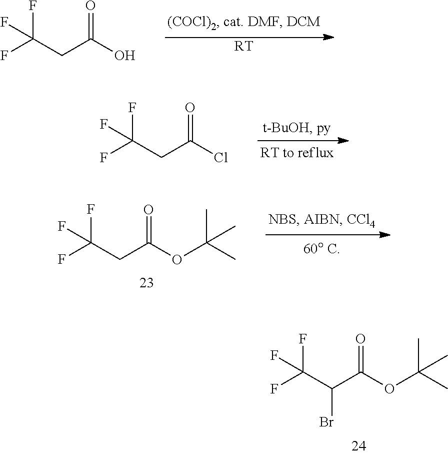

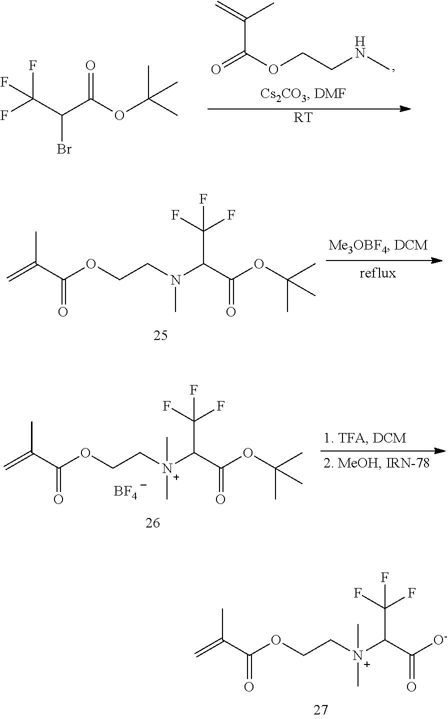

3,3-trifluoro-2-((2-(methacryloyloxy)ethyl)dimethylammonio)propanoate (27). 2-((3-(tert-butoxy)-1,1,1-trifluoro-3-oxopropan-2-yl)(methyl)amino)ethyl methacrylate (1.00 eq) was mixed with dry dichloromethane and trimethyloxonium tetrafluoroborate (2.00 eq) and stirred at reflux for 24 hours. The reaction was quenched with cold saturated sodium bicarbonate and extracted with dichloromethane. The solvent was removed in vacuo, and the residue dissolved in dichloromethane. Trifluoroacetic acid was added, and the reaction was stirred for 3 hours. Volatiles were removed and the crude compound was purified using a column of IRN-78 resin to give a white foam in an overall 83% yield. LC-MS (ESI, positive mode, m/z) calc. 284.11, found 284.10 for [M+H]⁺. ¹⁹F NMR (470 MHz, MeOD): δ −71.94 (s).

### Synthesis of Compound 5

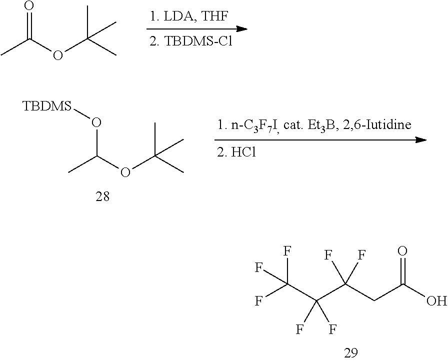

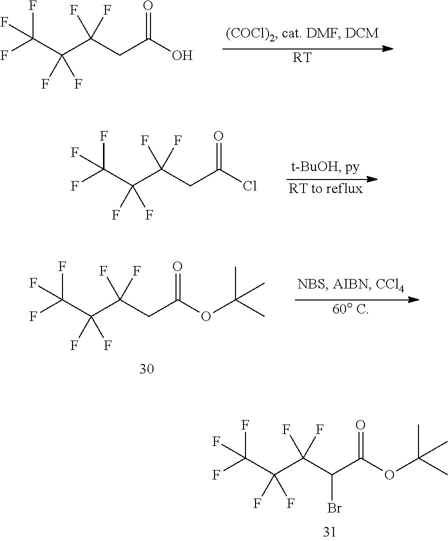

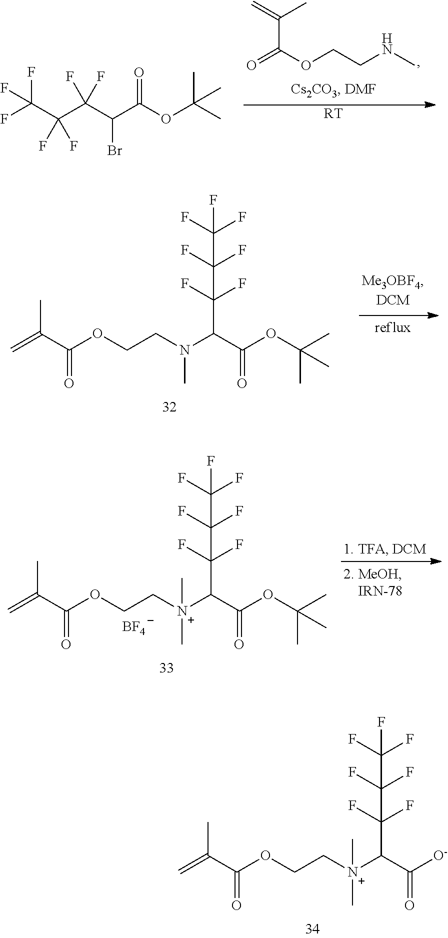

3,3,4,4,5,5,5-heptafluoro-2-((2-(methacryloyloxy)ethyl)dimethylammonio)-pentanoate (34). tert-butyl 3,3,4,4,5,5,5-heptafluoro-2-((2-(methacryloyloxy)ethyl)-(methyl)amino)pentanoate (1.00 eq) was mixed with dry dichloromethane and trimethyloxonium tetrafluoroborate (2.00 eq) and stirred at reflux for 24 hours. The reaction was quenched with cold saturated sodium bicarbonate and extracted with dichloromethane. The solvent was removed in vacuo, and the residue dissolved in dichloromethane. Trifluoroacetic acid was added, and the reaction was stirred for 3 hours. Volatiles were removed and the crude compound was purified using a column of IRN-78 resin to give a white foam in an overall 78% yield. LC-MS (ESI, positive mode, m/z) calc. 384.10, found 384.10 for [M+H]⁺. ¹⁹F NMR (470 MHz, MeOD): δ −66.40 (m), −124.49 (m), −125.85 (m).

### Synthesis of Compound 6

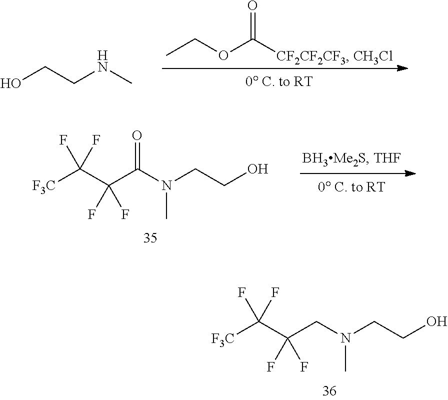

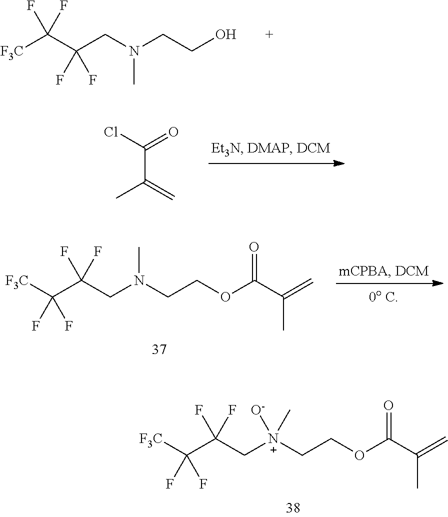

2,2,3,3,4,4,4-heptafluoro-N-(2-hydroxyethyl)-N-methylbutanamide (35). A flame dried 1 L Schlenk flask was charged with 2-(methylamino)ethan-1-ol (118.42 g) and cooled to −5° C. in an ice-salt bath. Ethyl 2,2,3,3,4,4,4-heptafluorobutanoate (381.67 g) was added dropwise. The reaction was allowed to come to room temperature over a period of one hour and stirred under N₂ for 20 hours. The ethanol byproduct was removed in vacuo. Yield was 422.56 g (99%) of a colorless oil.

2-((2,2,3,3,4,4,4-heptafluorobutyl)(methyl)amino)ethan-1-ol (36). To a flame dried 2 L 3-necked flask was added 1 L of freshly distilled anhydrous tetrahydrofuran and 2,2,3,3,4,4,4-heptafluoro-N-(2-hydroxyethyl)-N-methylbutanamide (375.00 g). The mixture was cooled to −5° C. in an ice-salt bath and borane dimethyl sulfide (150 mL) was added over 1.5 hours via dropping funnel. The reaction was allowed to come to room temperature and stirred under N₂ for 17 hours. The reaction was quenched with the addition of methanol. Volatiles were removed via rotary evaporation, and the crude was repeatedly subjected to dissolution in methanol and removal of volatiles in vacuo. The product was purified by fractional distillation under reduced pressure. Yield was 322.58 g (91%) of a colorless oil.

2-((2,2,3,3,4,4,4-heptafluorobutyl)(methyl)amino)ethyl methacrylate (37). To an oven dried 3-necked 1 L flask was added 400 mL anhydrous dichloromethane, 4-dimethylaminopyridine (5.21 g), freshly distilled anhydrous triethylamine (180 mL), and 2-((2,2,3,3,4,4,4-heptafluorobutyl)(methyl)amino)ethan-1-ol (109.63 g). The mixture was cooled to −5° C. in an ice-salt bath. Freshly distilled methacryloyl chloride (53.49 g) was added via syringe pump over 3 hours. The reaction was allowed to come to room temperature and stirred under N₂ for 12 hours. Methanol (50 mL) was added dropwise to quench the reaction. The mixture was filtered, the solvent was removed in vacuo, the residue was dissolved in tetrahydrofuran, and the resulting mixture was filtered again. Fractional distillation at reduced pressure yielded 117.42 g (85%) of a colorless oil.

2,2,3,3,4,4,4-heptafluoro-N-(2-(methacryloyloxy)ethyl)-N-methylbutan-1-amine oxide (38). To a 2 L oven dried 2-necked flask was added 2-((2,2,3,3,4,4,4-heptafluorobutyl)(methyl)amino)ethyl methacrylate (50.00 g) in 800 mL anhydrous dichloromethane. The mixture was cooled to −5° C. in an ice-salt bath. Purified meta-chloroperoxybenzoic acid (26.53 g) in 200 mL of anhydrous dichloromethane was added slowly via dropping funnel. The reaction was stirred for 2 hours under N₂ at −5° C. It was then concentrated to 50 mL and subjected to a plug of activated alumina, eluting several column volumes with dichloromethane and followed by 10% methanol:dichoromethane to afford the product. Volatiles were removed in vacuo to yield 48.89 g (93%) of an opaque, thick oil.

### Synthesis of Compound 7

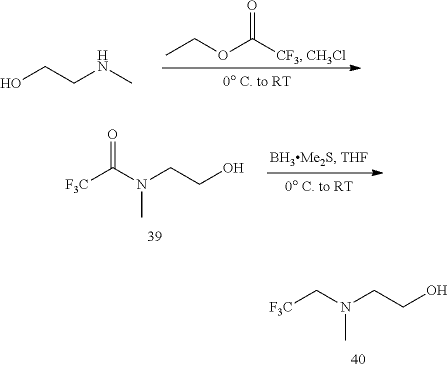

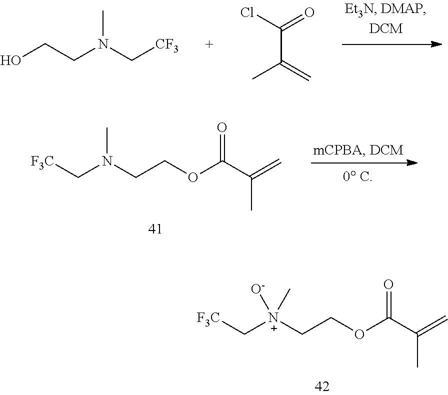

2,2,2-trifluoro-N-(2-hydroxyethyl)-N-methylacetamide (39). A flame dried 1 L Schlenk flask was charged with 2-(methylamino)ethan-1-ol (52.87 g) and cooled to −5° C. in an ice-salt bath. Ethyl trifluoroacetate (100.00 g) was added dropwise. The reaction was allowed to come to room temperature over a period of one hour and stirred under N₂ for 15 hours. The ethanol byproduct was removed in vacuo. Yield was 119.60 g (99%) of a colorless oil.

2-(methyl(2,2,2-trifluoroethyl)amino)ethan-1-ol (40). To a flame dried 2 L 3-necked flask was added 1 L of freshly distilled anhydrous tetrahydrofuran and 2,2,2-trifluoro-N-(2-hydroxyethyl)-N-methylacetamide (115.00 g). The mixture was cooled to −5° C. in an ice-salt bath and borane dimethyl sulfide (100 mL) was added over 1 hour via dropping funnel. The reaction was allowed to come to room temperature and stirred under N₂ for 12 hours. The reaction was quenched with the addition of methanol. Volatiles were removed via rotary evaporation, and the crude was repeatedly subjected to dissolution in methanol and removal of volatiles in vacuo. The product was purified by fractional distillation under reduced pressure. Yield was 97.47 g (92%) of a colorless oil.

2-(methyl(2,2,2-trifluoroethyl)amino)ethyl methacrylate (41). To an oven dried 3-necked 1 L flask was added 400 mL anhydrous dichloromethane, 4-dimethylamino-pyridine (5.21 g), freshly distilled anhydrous triethylamine (180 mL), and 2-(methyl(2,2,2-trifluoroethyl)amino)ethan-1-ol (67.00 g). The mixture was cooled to −5° C. in an ice-salt bath. Freshly distilled methacryloyl chloride (53.49 g) was added via syringe pump over 3 hours. The reaction was allowed to come to room temperature and stirred under N₂ for 10 hours. Methanol (50 mL) was added dropwise to quench the reaction. The mixture was filtered, the solvent was removed in vacuo, the residue was dissolved in tetrahydrofuran, and the resulting mixture was filtered again. Fractional distillation at reduced pressure yielded 84.69 g (88%) of a colorless oil.

2,2,2-trifluoro-N-(2-(methacryloyloxy)ethyl)-N-methylethan-1-amine oxide (42). To a 4 L oven dried 2-necked flask was added 2-(methyl(2,2,2-trifluoroethyl)amino)ethyl methacrylate (75.00 g) in 3000 mL anhydrous dichloromethane. The mixture was cooled to −5° C. in an ice-salt bath. Purified meta-chloroperoxybenzoic acid (74.75 g) in 500 mL of anhydrous dichloromethane was added slowly via dropping funnel. The reaction was stirred for 3 hours under N₂ at −5° C. It was then concentrated to 200 mL and subjected to a plug of activated alumina, eluting several column volumes with dichloromethane and followed by 5% methanol:dichoromethane to afford the product. Volatiles were removed in vacuo to yield 75.86 g (94%) of a white solid.

### Novel Route for Fluorinated Carboxybetaine (CB) Monomer

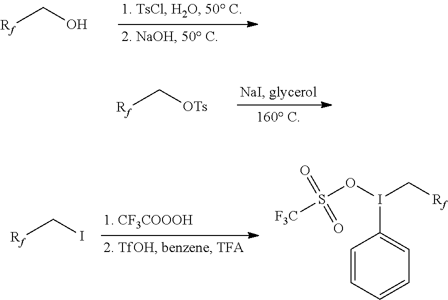

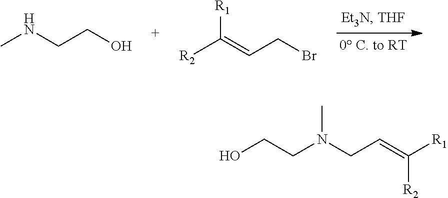

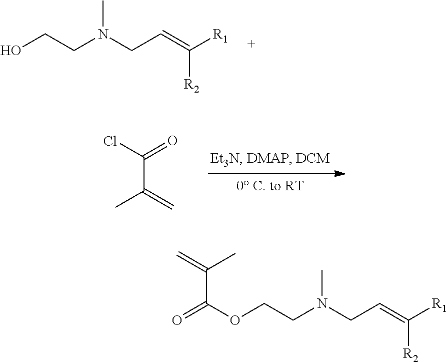

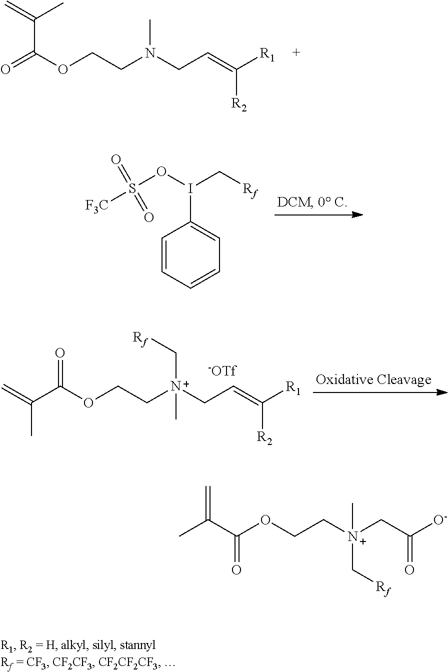

### Novel Process for Preparation of Intermediate for Production of CB Monomer

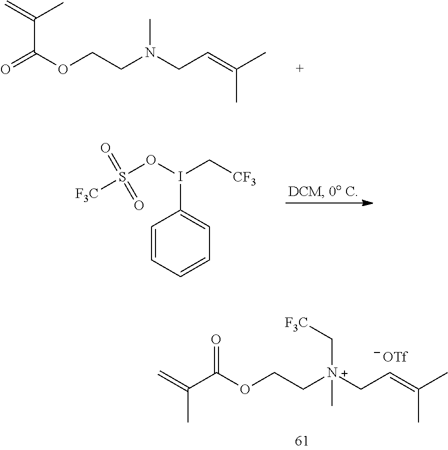 [SMILES: C=C(C)C(=O)OCCN(C)CC=C(C)C ; O=S(=O)(OI(CC(F)(F)F)c1ccccc1)C(F)(F)F ; C=C(C)C(=O)OCC[N+](C)(CC=C(C)C)CC(F)(F)F]

N-(2-(methacryloyloxy)ethyl)-N,3-dimethyl-N-(2,2,2-trifluoroethyl)but-2-en-1-aminium trifluoromethanesulfonate (61). A flame dried 2 L Schlenk flask was charged with 1 L dichloromethane and 2-(methyl(3-methylbut-2-en-1-yl)amino)ethyl methacrylate (50.00 g). The mixture was cooled to −5° C. in an ice-salt bath. Addition of phenyl(2,2,2-trifluoroethyl)-13-iodaneyl trifluoromethanesulfonate (103.19 g) proceeded slowly via solid addition funnel. The heterogenous, cloudy mixture was stirred until clarity was achieved after 1 hour. The volatiles were removed in vacuo, and the resulting oil was triturated with benzene 3 times. The oil was dried in vacuo to yield 100.82 g (96%) of a white foam.

### Novel Route for Fluorinated Trimethylammonium Oxide (TMAO) Monomer

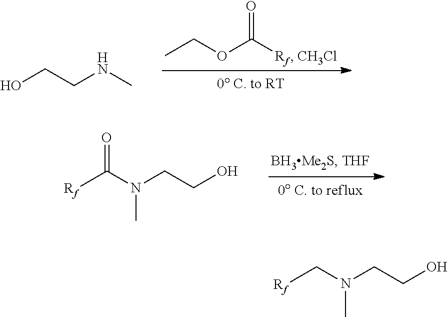

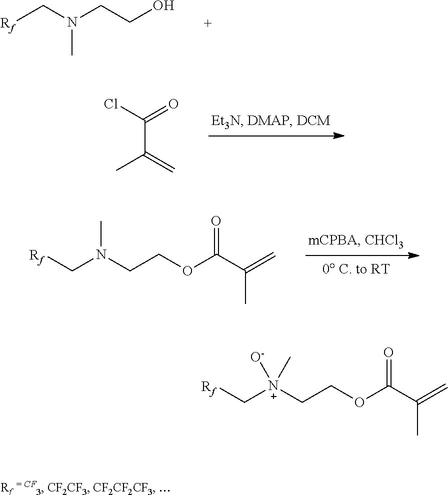

### Novel Process for Preparation of Intermediate for Production of TMAO Monomer

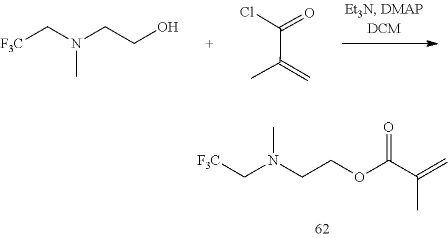

2-(methyl(2,2,2-trifluoroethyl)amino)ethyl methacrylate (62). To an oven dried 3-necked 1 L flask was added 400 mL anhydrous dichloromethane, 4-dimethylamino-pyridine (5.21 g), freshly distilled anhydrous triethylamine (180 mL), and 2-(methyl(2,2,2-trifluoroethyl)amino)ethan-1-ol (67.00 g). The mixture was cooled to −5° C. in an ice-salt bath. Freshly distilled methacryloyl chloride (53.49 g) was added via syringe pump over 3 hours. The reaction was allowed to come to room temperature and stirred under N₂ for 10 hours. Methanol (50 mL) was added dropwise to quench the reaction. The mixture was filtered, the solvent was removed in vacuo, the residue was dissolved in tetrahydrofuran, and the resulting mixture was filtered again. Fractional distillation at reduced pressure yielded 84.69 g (88%) of a colorless oil.

### Coating Example Using 2,2,2-Trifluoro-N-(2-(methacryloyloxy)ethyl)-N-methylethan-1-amine oxide

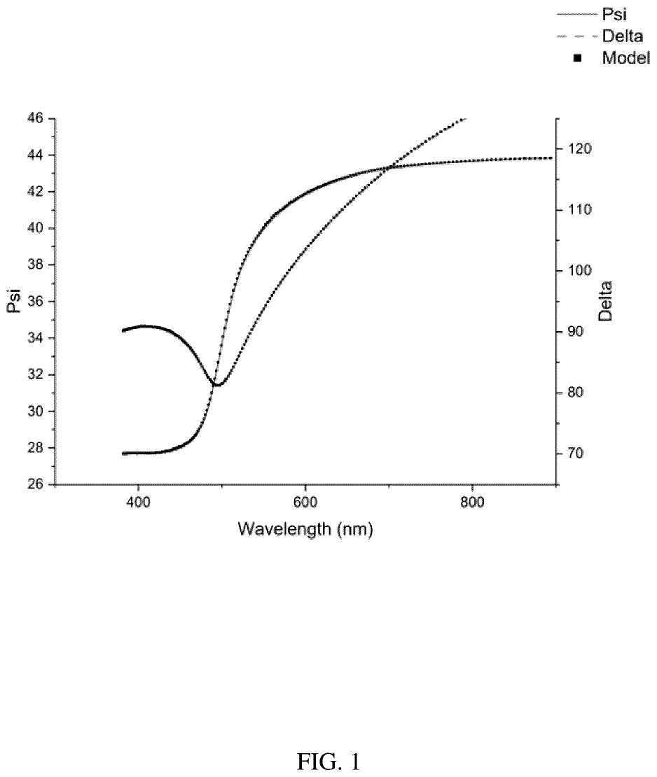

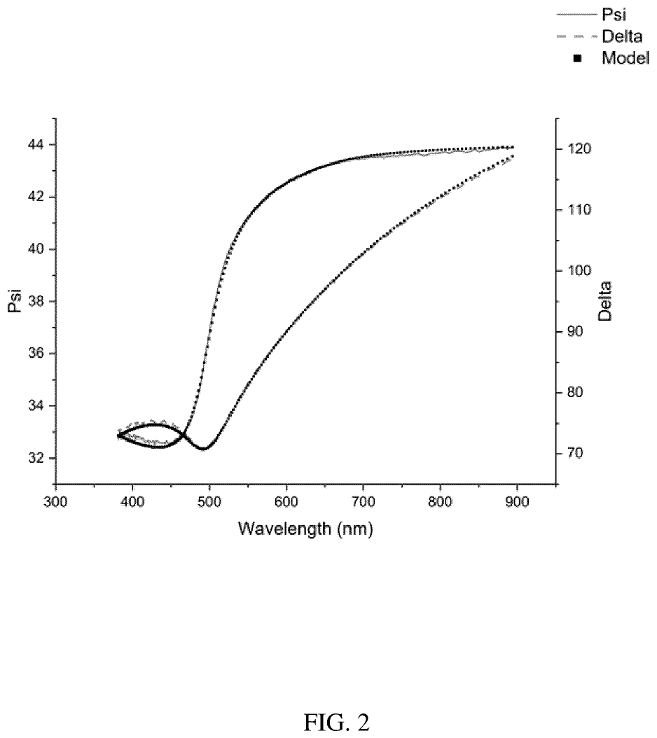

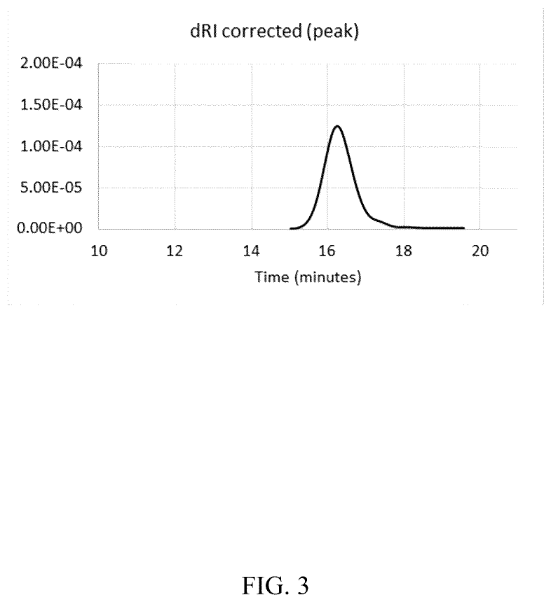

A gold-coated glass substrate was cleaned sequentially using ethanol, water, piranha solution (1 hour), water, and ethanol. The substrate was dried under a gentle stream of nitrogen and soaked in a 1 mM solution of bis[2-(2-bromo-isobutyryloxy)undecyl]disulfide for 3 hours in order to functionalize it with a self-assembled monolayer (SAM). It was subsequentially rinsed with ethanol thoroughly and dried under a stream of nitrogen. The thickness was measured using ellipsometry with a multilayer model and found to be 2.79 nm (FIG. 1). A 11:1 trifluoroethanol/water solution was used to dissolve 135 mg of 2,2,2-trifluoro-N-(2-(methacryloyloxy)ethyl)-N-methylethan-1-amine oxide, 0.20 mg of copper (II) chloride, and 2.76 mg of tris(2-pyridylmethyl)amine. The homogenous mixture was degassed via five cycles of freeze-pump-thaw. This mixture was then syringed into a microwave vial that was previously purged with nitrogen and contained the functionalized gold substrate. 10 mg of ascorbic acid in water was then added to start the polymerization. After 10 hours, the substrate was removed and thoroughly rinsed with trifluoroethanol and water. Its thickness was measured using ellipsometry with a multilayer model and found to be 11.86 nm (FIG. 2), suggesting the presence of polymer. The substrate was then added to a 5 mM iodine solution for 18 hours. The volatiles were removed under high vacuum with heating to 40° C. The resulting residue was dissolved in water and subjected to gel permeation chromatography using PBS buffer at a flow rate of 0.5 mL/min on a ultragel 250 column. The relative number average molecular weight was calculated and found to be 1096 with a dispersity of 1.23, thus confirming the success of the polymerization (FIG. 3).

While there have been shown and described what are at present considered the preferred embodiments of the invention, those skilled in the art may make various changes and modifications which remain within the scope of the invention defined by the appended claims.

What is claimed is:

1. A monomer composition having the formula: 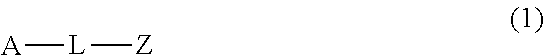
wherein:
A is a polymerizable group;
L is a bond or linking portion;
Z is a zwitterionic moiety or a zwitterionic precursor moiety containing a protecting group capable of deprotection to form a charged group;
wherein at least one hydrogen atom in A, L, and/or Z in Formula (1) is substituted by a fluorinated hydrocarbon group, wherein the fluorinated hydrocarbon group contains 1-12 carbon atoms and at least one fluorine atom.

2. The monomer composition of claim 1, having the following structure: 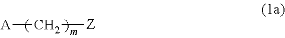
wherein A and Z are as defined in claim 1; and
m is an integer of at least 1;
wherein at least one hydrogen atom in A, CH₂, and/or Z in Formula (1a) is substituted by a fluorinated hydrocarbon group, wherein the fluorinated hydrocarbon group contains 1-12 carbon atoms and at least one fluorine atom.

3. The monomer composition of claim 2, having the following structure: 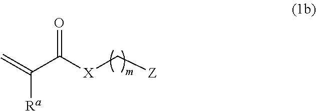
wherein:
R^{a} is H or an alkyl group containing 1-3 carbon atoms;
X is O or NR^{b}, wherein R^{b} is H or an alkyl group containing 1-3 carbon atoms;
Z is a zwitterionic moiety or a zwitterionic precursor moiety containing a protecting group capable of deprotection to form a charged group;
m is an integer of at least 1; and
at least one hydrogen atom in Formula (1b) is substituted by a fluorinated hydrocarbon group, wherein the fluorinated hydrocarbon group contains 1-12 carbon atoms and at least one fluorine atom.

4. The composition of any one of claims 1-3, wherein Z is selected from the group consisting of carboxybetaine, sulfobetaine, phosphobetaine, and trialkylamine-N-oxide zwitterionic moieties.

5. The composition of claim 3, wherein Z contains a positively charged group directly bound to a negatively charged group to result in the following structure: 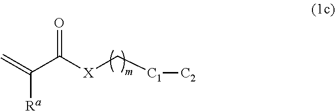
wherein R^{a}, X, and m are as defined in claim 3; and
C₁ and C₂ are independently selected as positively charged and negatively charged groups to form a zwitterionic moiety C₁-C₂;
provided that at least one hydrogen atom on R^{a}, X, methylene group under m, C₁, and/or C₂ is substituted by a fluorinated hydrocarbon group, wherein the fluorinated hydrocarbon group contains 1-12 carbon atoms and at least one fluorine atom.

6. The composition of claim 5, comprising the following structure: 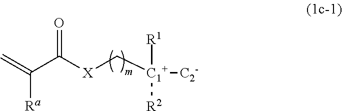
wherein R^{a}, X, and m are as defined in claim 3; and
R¹ and R² are independently selected from hydrocarbon groups containing 1-12 carbon atoms, wherein at least one of R¹ and R² contains at least one fluorine atom;
C₁⁺ and C₂⁻ are positively charged and negatively charged atoms or groups, respectively, to form a zwitterionic moiety C₁⁺-C₂⁻;
the dashed line represents an optional bond;
wherein any one or more of R^{a}, X, and methylene group under m is optionally substituted by fluorine or a fluorinated hydrocarbon group, wherein the fluorinated hydrocarbon group contains 1-12 carbon atoms and at least one fluorine atom.

7. The composition of claim 6, wherein Z is a trialkylamine-N-oxide zwitterionic moiety and has the following structure: 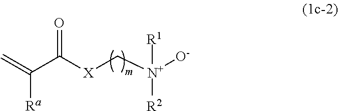
wherein R^{a}, X, and m are as defined in claim 3; and
R¹ and R² are independently selected from R^{a}, as defined in claim 3;
provided that at least one hydrogen atom on R^{a}, X, methylene group under m, R¹, and/or R² is substituted by a fluorinated hydrocarbon group, wherein the fluorinated hydrocarbon group contains 1-12 carbon atoms and at least one fluorine atom.

8. The composition of claim 6, comprising the following structure: 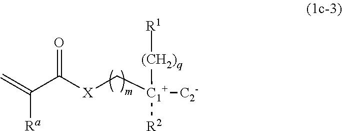
wherein R^{a}, X, and m are as defined in claim 3;
R² is selected from R^{a}, as defined in claim 3;
C₁⁺ and C₂⁻ are positively charged and negatively charged atoms or groups, respectively, to form a zwitterionic moiety C₁⁺-C₂⁻;
the dashed line represents an optional bond;
q is an integer of precisely or at least 1;
Rⁱ is a fluorinated hydrocarbon group containing 1-12 carbon atoms and at least one fluorine atom;
wherein any one or more of R^{a}, X, and methylene group under m is optionally substituted by fluorine or a fluorinated hydrocarbon group, wherein the fluorinated hydrocarbon group contains 1-12 carbon atoms and at least one fluorine atom.

9. The composition of claim 8, comprising the following structure 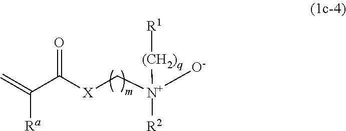
wherein R^{a}, X, and m are as defined in claim 3; and
R² is selected from R^{a}, as defined in claim 3;
q is an integer of precisely or at least 1;
Rⁱ is a fluorinated hydrocarbon group containing 1-12 carbon atoms and at least one fluorine atom;
wherein any one or more of R^{a}, X, methylene group under m, and/or R² is optionally substituted by fluorine or a fluorinated hydrocarbon group, wherein the fluorinated hydrocarbon group contains 1-12 carbon atoms and at least one fluorine atom.

10. The composition of any one of claims 1-3, wherein Z contains a positively charged group indirectly bound to a negatively charged group via a linker.

11. The composition of claim 10, wherein the olefin monomer composition has the following structure: 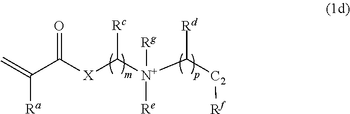
wherein R^{a}, X, and m are as defined in claim 3;
R^{c}, R^{d}, R^{e}, and R^{f} are independently selected from hydrogen atom, hydrocarbon group, and fluorinated hydrocarbon group containing 1-12 carbon atoms and at least one fluorine atom, wherein at least one of R^{c}, R^{d}, R^{e}, and R^{f} is said fluorinated hydrocarbon group;
one or both of R^{e} and R^{f} are alternatively selected from positive and negative charges;
C₁ and C₂ are independently selected as positively charged and negatively charged groups to form a zwitterionic moiety;
C₁ is optionally neutral and R^{e} is a protecting group capable of deprotection to form a charged group, or C₂ is optionally neutral and R^{f} is a protecting group capable of deprotection to form a charged group; and
p is an integer of at least 1.

12. The composition of claim 11, wherein the olefin monomer composition has the following structure: 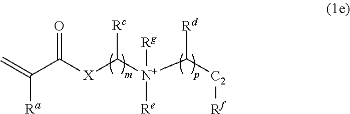
wherein R^{a}, X, and m are as defined in claim 3;
R^{c}, R^{d}, R^{e}, R^{f}, and R^{g} are independently selected from hydrogen atom, hydrocarbon group, and fluorinated hydrocarbon group containing 1-12 carbon atoms and at least one fluorine atom, wherein at least one of R^{c}, R^{d}, R^{e}, R^{f}, and R^{g} is said fluorinated hydrocarbon group;
R^{f} is alternatively a negative charge;
C₂ is a negatively charged group;
optionally, C₂ is neutral and R^{f} is a protecting group capable of deprotection to form a charged group; and
p is an integer of at least 1.

13. The composition of claim 12, wherein the olefin monomer composition has the following structure: 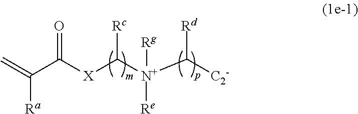
wherein R^{a}, X, and m are as defined in claim 3;
R^{c}, R^{d}, R^{e}, and R^{g} are independently selected from hydrogen atom, hydrocarbon group, and fluorinated hydrocarbon group containing 1-12 carbon atoms and at least one fluorine atom, wherein at least one of R^{c}, R^{d}, R^{e}, R^{f}, and R^{g} is said fluorinated hydrocarbon group;
C₂⁻ is a negatively charged group; and
p is an integer of at least 1.

14. The composition of claim 11, wherein the olefin monomer composition has the following structure: 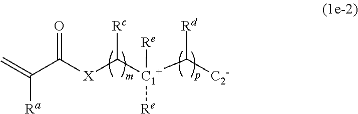
wherein R^{a}, X, and m are as defined in claim 3;
R^{c} and R^{d} are independently selected from hydrogen atom, hydrocarbon group, and fluorinated hydrocarbon group containing 1-12 carbon atoms and at least one fluorine atom;
each R^{e} is independently selected from hydrocarbon groups containing 1-12 carbon atoms and R^{a}, wherein at least one R^{e} contains at least one fluorine atom;
the dashed line represents an optional bond;
C₁⁺ is a positively charged atom or group;
C₂⁻ is a negatively charged atom or group; and
p is an integer of at least 1
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

15. The composition of claim 14, wherein the olefin monomer composition has the following structure: 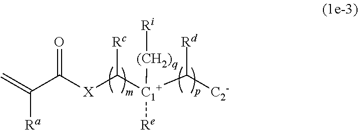
wherein:
R^{a}, X, and m are as defined in claim 3;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 3;
the dashed line represents an optional bond;
C₁⁺ is a positively charged atom or group;
C₂⁻ is a negatively charged atom or group;
q is an integer of precisely or at least 1;
Rⁱ is a fluorinated hydrocarbon group containing 1-12 carbon atoms and at least one fluorine atom;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

16. The composition of claim 15, wherein the olefin monomer composition has the following structure: 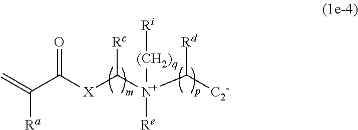
wherein:
R^{a}, X, and m are as defined in claim 3;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 3;
q is an integer of precisely or at least 1;
Rⁱ is a fluorinated hydrocarbon group containing 1-12 carbon atoms and at least one fluorine atom;
C₂⁻ is a negatively charged group;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

17. The composition of claim 16, wherein the olefin monomer composition has any of the following structures: 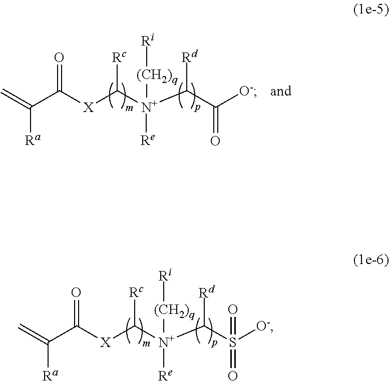
wherein:
R^{a}, X, and m are as defined in claim 3;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 3;
q is an integer of precisely or at least 1;
Rⁱ is a fluorinated hydrocarbon group containing 1-12 carbon atoms and at least one fluorine atom;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

18. The composition of claim 13, wherein the olefin monomer composition has any of the following structures: 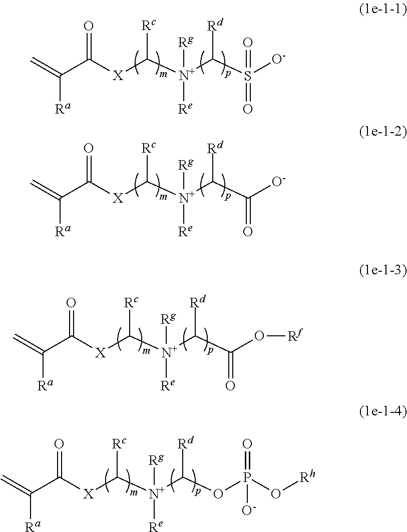
wherein R^{a}, X, and m are as defined in claim 3;
R^{c}, R^{d}, R^{e}, R^{g}, and R^{h} are independently selected from hydrogen atom, hydrocarbon group, and fluorinated hydrocarbon group containing 1-12 carbon atoms and at least one fluorine atom, wherein at least one of R^{c}, R^{d}, R^{e}, R^{f}, R^{g}, and R^{h} is said fluorinated hydrocarbon group;
R^{f} is optionally a protecting group capable of deprotection to form a carboxylate group; and
p is an integer of at least 1.

19. The composition of claim 11, wherein the olefin monomer composition has the following structure: 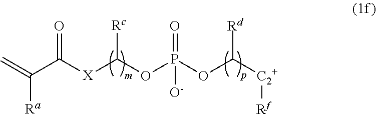
wherein R^{a}, X, and m are as defined in claim 3;
C₂⁺ is a positively charged group;
p is an integer of at least 1; and
R^{c}, R^{d}, and R^{f} are independently selected from hydrogen atom, hydrocarbon group, and fluorinated hydrocarbon group containing 1-12 carbon atoms and at least one fluorine atom, wherein at least one of R^{c}, R^{d}, and R^{f} is said fluorinated hydrocarbon group;
wherein R^{f} is alternatively absent.

20. The composition of claim 19, wherein the olefin monomer composition has the following structure: 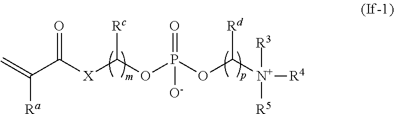
wherein R^{a}, X, and m are as defined in claim 1;
p is an integer of at least 1; and
R^{c}, R^{d}, R³, R⁴, and R⁵ are independently selected from hydrogen atom, hydrocarbon group, and fluorinated hydrocarbon group containing 1-12 carbon atoms and at least one fluorine atom, wherein at least one of R^{c}, R^{d}, R³, R⁴, and R⁵ is said fluorinated hydrocarbon group.

21. A zwitterionic polymer comprising the formula: 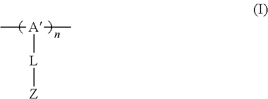
wherein:
A′ is a backbone of the polymer formed by polymerization of A;
L is a bond or linking portion;
Z is a zwitterionic moiety or a zwitterionic precursor moiety containing a protecting group capable of deprotection to form a charged group;
n is an integer of at least 2;
wherein at least one hydrogen atom in A′, L, and/or Z in Formula (I) is substituted by a fluorinated hydrocarbon group, wherein the fluorinated hydrocarbon group contains 1-12 carbon atoms and at least one fluorine atom;
and copolymers of Formula (I).

22. The zwitterionic polymer of claim 21, wherein the zwitterionic polymer has the following structure: 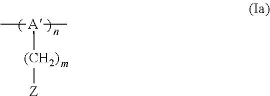
wherein:
A′ and Z are as defined in claim 14;
n is an integer of at least 2; and
m is an integer of at least 1.

23. The zwitterionic polymer of claim 22, wherein the zwitterionic polymer has the following structure: 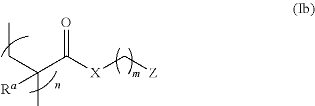
wherein:
R^{a} is H or an alkyl group containing 1-3 carbon atoms;
X is O or NR^{b}, wherein R^{b} is H or an alkyl group containing 1-3 carbon atoms;
Z is a zwitterionic moiety or a zwitterionic precursor moiety containing a protecting group capable of deprotection to form a charged group;
n is an integer of at least 2; and
m is an integer of at least 1;
wherein at least one hydrogen atom in Formula (Ia) is substituted by a fluorinated hydrocarbon group, wherein the fluorinated hydrocarbon group contains 1-12 carbon atoms and at least one fluorine atom.

24. The composition of any one of claims 21-23, wherein Z is selected from the group consisting of carboxybetaine, sulfobetaine, phosphobetaine, and trialkylamine-N-oxide zwitterionic moieties.

25. The composition of claim 23, wherein Z contains a positively charged group directly bound to a negatively charged group to result in the following structure: 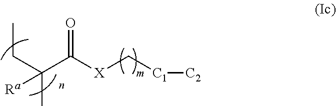
wherein R^{a}, X, n, and m are as defined in claim 23; and
C₁ and C₂ are independently selected as positively charged and negatively charged groups to form a zwitterionic moiety C₁-C₂;
provided that at least one hydrogen atom on R^{a}, X, methylene group under m, C₁, and/or C₂ is substituted by a fluorinated hydrocarbon group, wherein the fluorinated hydrocarbon group contains 1-12 carbon atoms and at least one fluorine atom.

26. The composition of claim 25, comprising the following structure: 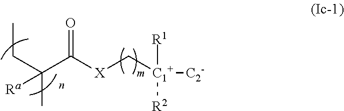
wherein R^{a}, X, and m are as defined in claim 23; and
R¹ and R² are independently selected from hydrocarbon groups containing 1-12 carbon atoms, wherein at least one of R¹ and R² contains at least one fluorine atom;
the dashed line represents an optional bond;
wherein any one or more of R^{a}, X, and methylene group under m is optionally substituted by fluorine or a fluorinated hydrocarbon group, wherein the fluorinated hydrocarbon group contains 1-12 carbon atoms and at least one fluorine atom.

27. The composition of claim 26, wherein Z is a trialkylamine-N-oxide zwitterionic moiety and the polymer has the following structure: 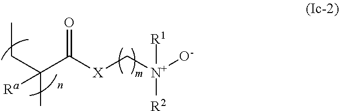
wherein R^{a}, X, n, and m are as defined in claim 23; and
R¹ and R² are independently selected from R^{a}, as defined in claim 12;
provided that at least one hydrogen atom on R^{a}, X, methylene group under m, R¹, and/or R² is substituted by a fluorinated hydrocarbon group, wherein the fluorinated hydrocarbon group contains 1-12 carbon atoms and at least one fluorine atom.

28. The composition of claim 26, comprising the following structure: 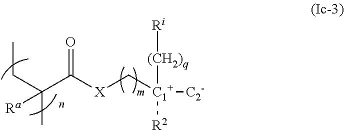
wherein R^{a}, X, and m are as defined in claim 23; and
R² is selected from R^{a}, as defined in claim 23;
the dashed line represents an optional bond;
q is an integer of precisely or at least 1;
Rⁱ is a fluorinated hydrocarbon group containing 1-12 carbon atoms and at least one fluorine atom;
wherein any one or more of R^{a}, X, and methylene group under m is optionally substituted by fluorine or a fluorinated hydrocarbon group, wherein the fluorinated hydrocarbon group contains 1-12 carbon atoms and at least one fluorine atom.

29. The composition of claim 28, comprising the following structure: 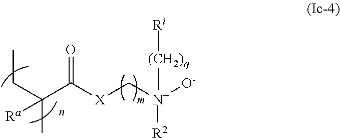
wherein R^{a}, X, and m are as defined in claim 23; and
R² is selected from R^{a}, as defined in claim 23;
q is an integer of precisely or at least 1;
Rⁱ is a fluorinated hydrocarbon group containing 1-12 carbon atoms and at least one fluorine atom;
wherein any one or more of R^{a}, X, methylene group under m, R¹, and/or R² is optionally substituted by fluorine or a fluorinated hydrocarbon group, wherein the fluorinated hydrocarbon group contains 1-12 carbon atoms and at least one fluorine atom.

30. The composition of any one of claims 21-23, wherein Z contains a positively charged group indirectly bound to a negatively charged group via a linker.

31. The composition of claim 30, wherein the composition has the following structure: 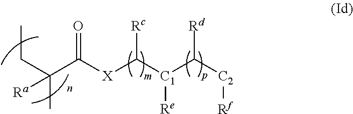
wherein R^{a}, X, n, and m are as defined in claim 23;
R^{c}, R^{d}, R^{e}, and R^{f} are independently selected from hydrogen atom, hydrocarbon group, and fluorinated hydrocarbon group containing 1-12 carbon atoms and at least one fluorine atom, wherein at least one of R^{c}, R^{d}, R^{e}, and R^{f} is said fluorinated hydrocarbon group;
one or both of R^{e} and R^{f} are alternatively selected from positive and negative charges;
C₁ and C₂ are independently selected as positively charged and negatively charged groups to form a zwitterionic moiety;
C₁ is optionally neutral and R^{e} is a protecting group capable of deprotection to form a charged group, or C₂ is optionally neutral and R^{f} is a protecting group capable of deprotection to form a charged group; and
p is an integer of at least 1.

32. The composition of claim 31, wherein the composition has the following structure: 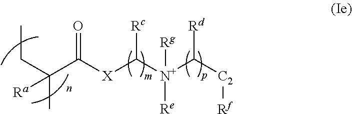
wherein R^{a}, X, n, and m are as defined in claim 23;
R^{c}, R^{d}, R^{e}, R^{f}, and R^{g} are independently selected from hydrogen atom, hydrocarbon group, and fluorinated hydrocarbon group containing 1-12 carbon atoms and at least one fluorine atom, wherein at least one of R^{c}, R^{d}, R^{e}, R^{f}, and R^{g} is said fluorinated hydrocarbon group;
R^{f} is alternatively a negative charge;
C₂ is a negatively charged group;
optionally, C₂ is neutral and R^{f} is a protecting group capable of deprotection to form a charged group; and
p is an integer of at least 1.

33. The composition of claim 32, wherein the composition has the following structure: 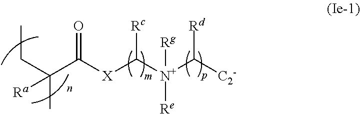
wherein R^{a}, X, n, and m are as defined in claim 23;
R^{c}, R^{d}, R^{e}, and R^{g} are independently selected from hydrogen atom and fluorinated hydrocarbon group containing 1-12 carbon atoms and at least one fluorine atom, wherein at least one of R^{c}, R^{d}, R^{e}, R^{f}, and R^{g} is said fluorinated hydrocarbon group;
C₂⁻ is a negatively charged group; and
p is an integer of at least 1.

34. The composition of claim 31, wherein the composition has the following structure: 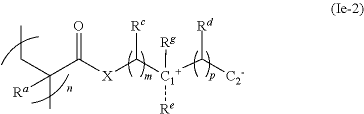
wherein R^{a}, X, and m are as defined in claim 23;
R^{c} and R^{d} are independently selected from hydrogen atom, hydrocarbon group, and fluorinated hydrocarbon group containing 1-12 carbon atoms and at least one fluorine atom;
each R^{e} is independently selected from hydrocarbon groups containing 1-12 carbon atoms and R^{a}, wherein at least one R^{e} contains at least one fluorine atom;
the dashed line represents an optional bond;
C₂⁻ is a negatively charged group; and
p is an integer of at least 1
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

35. The composition of claim 34, wherein the composition has the following structure: 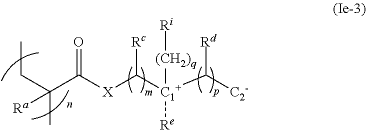
wherein:
R^{a}, X, and m are as defined in claim 23;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 23;
the dashed line represents an optional bond;
q is an integer of precisely or at least 1;
Rⁱ is a fluorinated hydrocarbon group containing 1-12 carbon atoms and at least one fluorine atom;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

36. The composition of claim 35, wherein the composition has the following structure: 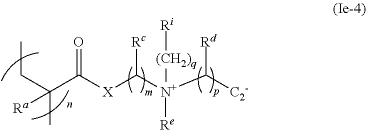
wherein:
R^{a}, X, and m are as defined in claim 23;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 23;
q is an integer of precisely or at least 1;
Rⁱ is a fluorinated hydrocarbon group containing 1-12 carbon atoms and at least one fluorine atom;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

37. The composition of claim 36, wherein the composition has any of the following structures: 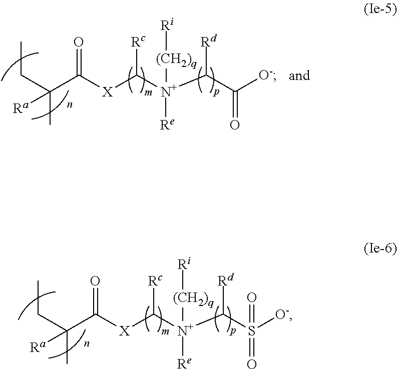
wherein:
R^{a}, X, and m are as defined in claim 3;
R^{c} and R^{d} are independently selected from hydrogen atom, hydrocarbon group, and fluorinated hydrocarbon group containing 1-12 carbon atoms and at least one fluorine atom;
R^{e} is selected from R^{a}, as defined in claim 3;
q is an integer of precisely or at least 1;
Rⁱ is a fluorinated hydrocarbon group containing 1-12 carbon atoms and at least one fluorine atom;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

38. The composition of claim 23, wherein the composition has any of the following structures: 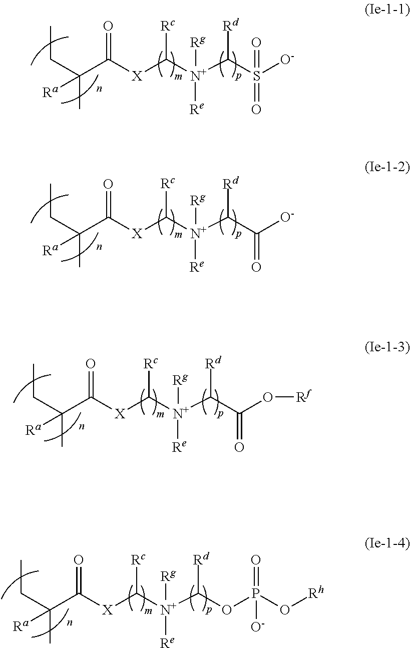
wherein R^{a}, X, n, and m are as defined in claim 23;
R^{c}, R^{d}, R^{e}, R^{f}, R^{g}, and R^{h} are independently selected from hydrogen atom and fluorinated hydrocarbon group containing 1-12 carbon atoms and at least one fluorine atom, wherein at least one of R^{c}, R^{d}, R^{e}, R^{f}, R^{g}, and R^{h} is said fluorinated hydrocarbon group; and
p is an integer of at least 1.

39. The composition of claim 31, wherein the composition has the following structure: 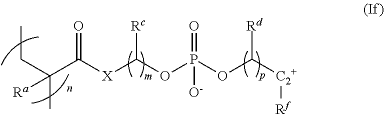
wherein R^{a}, X, n, and m are as defined in claim 23;
C₂⁺ is a positively charged group;
p is an integer of at least 1; and
R^{c}, R^{d}, and R^{f} are independently selected from hydrogen atom, hydrocarbon group, and fluorinated hydrocarbon group containing 1-12 carbon atoms and at least one fluorine atom, wherein at least one of R^{c}, R^{d}, and R^{f} is said fluorinated hydrocarbon group;
wherein R^{f} is alternatively absent.

40. The composition of claim 39, wherein the composition has the following structure: 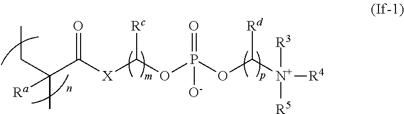
wherein R^{a}, X, n, and m are as defined in claim 23;
p is an integer of at least 1; and
R^{c}, R^{d}, R³, R⁴, and R⁵ are independently selected from hydrogen atom and fluorinated hydrocarbon group containing 1-12 carbon atoms and at least one fluorine atom, wherein at least one of R^{c}, R^{d}, R³, R⁴, and R⁵ is said fluorinated hydrocarbon group.

41. A mixed-charge zwitterionic copolymer comprising the following formula: 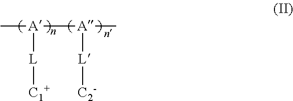
wherein:
A′ and A″ are backbones of different copolymer segments shown in Formula (II);
L and L′ are bonds and/or linking portions;
C₁⁺ is a positively charged group, and C₂⁻ is a negatively charged group, wherein C₁⁺ and C₂⁻ together form a zwitterionic system;
n and n′ are independently selected from integers of at least 2;
wherein at least one hydrogen atom in A′, A″, L, L′, C₁⁺, and/or C₂⁻ in Formula (I) is substituted by a fluorinated hydrocarbon group, wherein the fluorinated hydrocarbon group contains 1-12 carbon atoms and at least one fluorine atom.

42. A hydrogel composition comprising a network entanglement of one, two or three zwitterionic polymers selected from any of the polymer compositions of claims 21-41.

43. The hydrogel composition of claim 42, wherein the hydrogel composition is a zwitterionic double-network (ZDN) hydrogel composition.

44. The hydrogel composition of claim 42, wherein the hydrogel composition is a zwitterionic triple-network (ZTN) hydrogel composition.

45. A hybrid copolymer composition comprising a copolymer resulting from polymerization of any one or more zwitterionic monomers of any one of claims 1-20 in combination with one or more non-zwitterionic monomers.

46. The hybrid copolymer composition of claim 45, wherein the hybrid copolymer is a random, block, alternating, branched, or star polymer.

47. The hybrid copolymer composition of any one of claims 45-46, wherein the non-zwitterionic monomer is a zwitterionic-modified version of an olefinic, polysiloxane, polyester, polyurethane, polyurea, polycarbonate, polypeptide, polyimide, polyphosphazene, polyepoxy, phenolic polymer, polysulfone, or polysulfide polymer.

48. A hybrid copolymer composition comprising a zwitterionic polymeric block having any of the compositions of any one of claims 21-41 grafted onto a non-zwitterionic polymer.

49. The hybrid copolymer composition of claim 48, wherein the non-zwitterionic polymer is selected from the group consisting of polyolefins (e.g., polyethylene or propylene), polysiloxanes, polyesters, polyurethanes, polyureas, polycarbonates, polypeptides, polyimides, polyphosphazenes, polyepoxies, phenolic polymers, polysulfones, and polysulfide polymers.

50. A crosslinked hydrogel composition comprising a crosslinked version of one, two, or three zwitterionic polymers or hybrid copolymers of any one of claims 21-49.

51. A method for rendering a surface fouling-resistant and/or fouling releasing, the method comprising coating the surface with any one, two, or three of the polymer, hybrid copolymer, or hydrogel compositions of claims 21-50.

52. A fouling-resistant and/or fouling release object comprising:
(i) an object having a surface; and
(ii) a coating comprising any one, two, or three of the polymer, hybrid copolymer, or hydrogel compositions of claims 21-50 on said surface.

53. The fouling-resistant and/or fouling release object of claim 52, wherein the coating possesses both a fouling-resistant and a fouling-release ability.

54. The fouling-resistant and/or fouling release object of claim 52, wherein the object is designed to operate in a marine environment or medical environment.

55. A zwitterionic nanoparticle composition comprising one, two, or three of the polymer, hybrid copolymer, or hydrogel compositions of claims 21-50 within and/or on surfaces of the nanoparticles.

56. A method of producing a carboxybetaine monomer of the formula 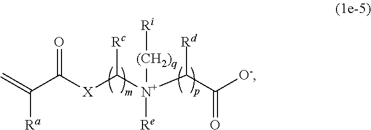
the method comprising:
(i) producing an intermediate (Int-1) by the following reaction scheme: 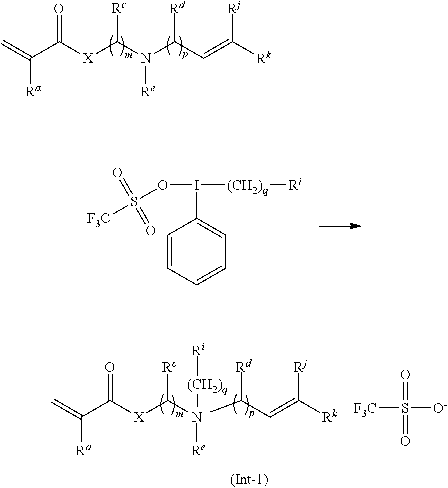
wherein:
R^{a} is H or an alkyl group containing 1-3 carbon atoms;
X is O or NR^{b}, wherein R^{b} is H or an alkyl group containing 1-3 carbon atoms;
m is an integer of precisely or at least 1;
p is an integer of precisely or at least 1;
R^{c} and R^{d} are independently selected from hydrogen atom, hydrocarbon group, and fluorinated hydrocarbon group containing 1-12 carbon atoms and at least one fluorine atom;
R^{e} is selected from R^{a};
q is an integer of precisely or at least 1;
Rⁱ is a fluorinated hydrocarbon group containing 1-12 carbon atoms and at least one fluorine atom; and
R^{j} and R^{k} are independently selected from hydrogen atom, alkyl group containing 1-3 carbon atoms, silyl groups, and stannyl groups;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom;
and
(ii) oxidatively cleaving the intermediate (Int-1) to produce the carboxybetaine monomer of the formula (1e-5).

57. An intermediate compound of the formula: 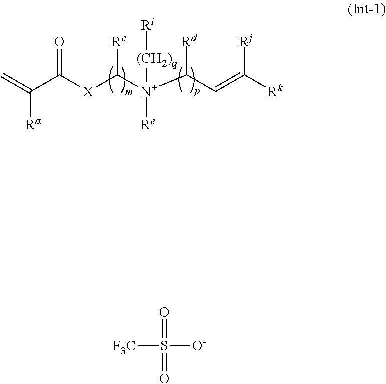
R^{a} is H or an alkyl group containing 1-3 carbon atoms;
X is O or NR^{b}, wherein R^{b} is H or an alkyl group containing 1-3 carbon atoms;
m is an integer of precisely or at least 1;
p is an integer of precisely or at least 1;
R^{c} and R^{d} are independently selected from hydrogen atom, hydrocarbon group, and fluorinated hydrocarbon group containing 1-12 carbon atoms and at least one fluorine atom;
R^{e} is selected from R^{a};
q is an integer of precisely or at least 1;
Rⁱ is a fluorinated hydrocarbon group containing 1-12 carbon atoms and at least one fluorine atom; and
R^{j} and R^{k} are independently selected from hydrogen atom, alkyl group containing 1-3 carbon atoms, silyl groups, and stannyl groups;
wherein any one or more of R^{a}, X, methylene group under m, R^{c}, and R^{d} is optionally substituted by fluorine or a fluorinated hydrocarbon group, wherein the fluorinated hydrocarbon group contains 1-12 carbon atoms and at least one fluorine atom.

58. A method of producing a trimethylammonium oxide (TMAO) monomer of the formula: 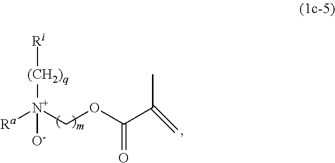
the method comprising:
(i) producing an intermediate (Int-2) by the following reaction scheme: 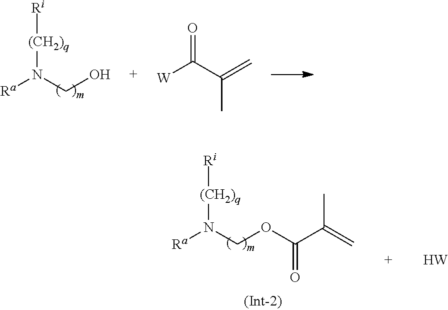
wherein:
R^{a} is H or an alkyl group containing 1-3 carbon atoms;
m is an integer of precisely or at least 1;
q is an integer of precisely or at least 1;
Rⁱ is a fluorinated hydrocarbon group containing 1-12 carbon atoms and at least one fluorine atom; and
W is Cl or Br;
and
(ii) oxidizing the intermediate (Int-2) to produce the TMAO monomer of the formula (1c-5).

59. An intermediate compound of the formula: 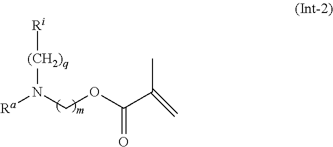
wherein:
R^{a} is H or an alkyl group containing 1-3 carbon atoms;
m is an integer of precisely or at least 1;
q is an integer of precisely or at least 1; and
Rⁱ is a fluorinated hydrocarbon group containing 1-12 carbon atoms and at least one fluorine atom.
